(19) United States
(12) Patent Application Publication
(10) Pub. No.: US 2023/0054895 A1
(43) Pub. Date: Feb. 23, 2023

(54) METHODS FOR INDUCING BIOSTASIS IN A CELL, TISSUE OR ORGAN

(71) Applicants: PRESIDENT AND FELLOWS OF HARVARD COLLEGE, Cambridge, MA (US); TRUSTEES OF TUFTS COLLEGE, Boston, MA (US)

(72) Inventors: Takako TAKEDA, Cambridge, MA (US); Megan Sperry, Cambridge, MA (US); Erica Gardner, Cambridge, MA (US); Michael Levin, Cambridge, MA (US); Charles Reilly, Cambridge, MA (US); Richard Novak, Jamaica Plain, MA (US); Donald E. Ingber, Boston, MA (US)

(73) Assignees: PRESIDENT AND FELLOWS OF HARVARD COLLEGE, Cambridge, MA (US); TRUSTEES OF TUFTS COLLEGE, Boston, MA (US)

(21) Appl. No.: 17/791,775

(22) PCT Filed: Jan. 8, 2021
(86) PCT No.: PCT/US2021/012626

§ 371 Date: Jul. 8, 2022

Related U.S. Application Data
(60) Provisional application No. 63/020,475, filed on May 5, 2020.
(60) Provisional application No. 62/959,372, filed on Jan. 10, 2020.

(51) Int. Cl. A01N 1/02; A61K 48/00

(52) U.S. Cl. CPC A01N 1/0226; A61K 48/00

(57) ABSTRACT

Provided herein are methods for promoting biostasis or preservation of a cell, tissue or organ during cancer treatment or for transplantation comprising contacting the cell, tissue or organ with an agonist of the δ-opioid receptor, SNC-80, an or Donepezil. Further provided herein is a method of treating a hematological neoplastic disease.

### CROSS-REFERENCE TO RELATED APPLICATIONS

This application claims benefit under 35 U.S.C. § 119(e) of U.S. Provisional Application No. 62/959,372 filed Jan. 10, 2020; and 63/020,475 filed May 5, 2020, the contents of which are incorporated herein by reference in their entirety.

### GOVERNMENT SUPPORT

This invention was made with Government Support under Contract No. W911NF1920027 awarded by the Defense Advanced Research Projects Agency (DARPA). The Government has certain rights in the invention.

### TECHNICAL FIELD

The technology described herein relates to methods for inducing stasis, or preserving a cell, tissue or organ.

### BACKGROUND

Time is often the limiting factor that governs, e.g., whether patients will be able to be timely transported to specialized care facilities and thus, survive a life-threatening injury. Developing therapeutics that extend this window for effective medical interventions from minutes to hours or days by inducing a chemical state of “suspended animation” would therefore have a dramatic impact on patient survival (Hadj-Moussa and Storey. The FEBS Journal. October 2018).

Prior attempts at addressing this challenge of extending the window for medical intervention have included applying external cooling of organs, limbs, and patients. This is now part of standard of care in certain surgeries, particularly where there is risk of ischemic damage to the brain. However, this method does not fully address this challenge and requires complex hardware and invasive connections to the vasculature.

Studies of ex vivo organ preservation have led to improved solutions for perfusion, flushing, and storage of organs, including the most commonly used University of Wisconsin solution. Perfluorocarbon solutions, which offer excellent oxygen transport properties, have also been implemented with some success. While hypothermic maintenance of organs ex vivo improves some organ transplant outcome by lowering metabolic rate, not all organs benefit from hypothermia. In fact, the imbalance between ATP production and ATP consumption triggered by hypothermia leads to worse transplant outcomes in some scenarios.

Thus, the ultimate need is to develop an agent that, when administered to a patient or organ/tissue, will rapidly and reversibly induce biostasis (including suppressed oxygen utilization and metabolism) in order to slow biological time and therefore prevent tissue degradation and damage.

### SUMMARY

Described herein is the discovery that SNC-80 induces a torpor-like hypometabolic state in cell culture and whole animal models. In whole animals, movement was completely arrested following high-dose administration of SNC-80. Importantly, it was found that the torpor-like effect on the whole animal was reversible; the movement arrest was subsequently reversed upon withdrawal of the drug. Thus, in some embodiments herein, SNC-80 is contacted with cells, tissues and/or organs to suppress metabolism in a stable, reversible manner for stabilization of cells, tissues, organs, and/or whole organisms, e.g., for transplantation.

Further, data presented herein show that SNC-80 slows metabolism of cultured intestinal cancer cells (Caco-2 cells) as measured by a reduction in intracellular ATP levels, but did not inhibit oxygen utilization. Thus, in some embodiments herein, SNC-80 can be used to protect normal cells from anti-cancer therapies, such as radiation and chemotherapies that induce oxygen free radical generation, and thereby increase the efficacy of such anti-cancer therapies and/or limiting collateral damage to non-cancer cells, tissues and organs.

One aspect described herein provides a method of inducing biostasis in a cell, tissue or organ, the method comprising contacting the cell, tissue or organ in need of preservation with an agent that alters the function of at least one ion channel selected from the group consisting of EAAT1 ion channel and NCX1 ion channel, wherein the contacted cell, tissue or organ exhibits biostasis.

Another aspect described herein provides a method of preserving viability or function of a cell, tissue or organ, the method comprising contacting the cell, tissue or organ in need of such preservation with an agent that alters the function of at least one ion channel selected from the group consisting of EAAT1 ion channel and NCX1 ion channel.

Another aspect described herein provides a method of cell, tissue or organ transplant, the method comprising contacting a donor cell, tissue or organ in situ or ex vivo with an agent that alters the function of at least one ion channel selected from the group consisting of EAAT1 ion channel and NCX1 ion channel.

Another aspect described herein provides a method of preparing a cell, tissue or organ for transplant, the method comprising contacting a donor cell, tissue or organ with an agent that alters the function of at least one ion channel selected from the group consisting of EAAT1 ion channel and NCX1 ion channel.

Another aspect described herein provides a method of inducing biostasis in a cell, tissue or organ, the method comprising contacting the cell, tissue or organ in need of preservation with an agonist for the δ-opioid receptor, wherein the contacted cell, tissue or organ exhibits biostasis, wherein the agonist is administered at a dose sufficient to alter the function of at least one ion channel selected from the group consisting of EAAT1 ion channel and NCX1 ion channel.

Another aspect described herein provides a method of preserving viability or function of a cell, tissue or organ, the method comprising contacting the cell, tissue or organ in need of such preservation with an agonist of the δ-opioid receptor, wherein the agonist is administered at a dose sufficient to alter the function of at least one ion channel selected from the group consisting of EAAT1 ion channel and NCX1 ion channel.

Another aspect described herein provides a method of cell, tissue or organ transplant, the method comprising contacting a donor cell, tissue or organ in situ or ex vivo with an agonist of the δ-opioid receptor, wherein the agonist is administered at a dose sufficient to alter the function of at least one ion channel selected from the group consisting of EAAT1 ion channel and NCX1 ion channel.

Another aspect described herein provides a method of preparing a cell, tissue or organ for transplant, the method comprising contacting a donor cell, tissue or organ with an agonist of the δ-opioid receptor, wherein the agonist is administered at a dose sufficient to alter the function of at least one ion channel selected from the group consisting of EAAT1 ion channel and NCX1 ion channel.

In one embodiment of any aspect provided herein, altering the function is inhibiting the function. In one embodiment of any aspect provided herein, altering the function is slowing the function. In one embodiment of any aspect provided herein, altering the function is activating the function

In one embodiment of any aspect provided herein, the tissue is an endoderm tissue, a mesoderm tissue, or an ectoderm tissue.

In one embodiment of any aspect provided herein, the tissue is selected from the group consisting of cornea, bone, cartilage, tendon, pancreas islet, heart valve, nerve, vascular, deep tissue flap, fat tissue, muscle, and vein.

In one embodiment of any aspect provided herein, the organ is selected from the group consisting of intestine, stomach, heart, kidney, bladder, pancreas, liver, lung, brain, skin, uterus, digit, and limb.

In one embodiment of any aspect provided herein, the contacting suppresses the metabolism or induces biostasis of the cell, tissue or organ.

In one embodiment of any aspect provided herein, the agent is SNC-80 or donepezil. In one embodiment of any aspect provided herein, the agent is a derivative, analog, or variant of SNC-80 that alters the function of at least one ion channel selected from the group consisting of EAAT1 ion channel and NCX1 ion channel.

In one embodiment of any aspect provided herein, the agent is a derivative, analog, or variant of donepezil that alters the function of at least one ion channel selected from the group consisting of EAAT1 ion channel and NCX1 ion channel.

In one embodiment of any aspect provided herein, the agonist is a derivative, analog, or variant of SNC-80 that activates signaling by the δ-opioid receptor and alters the function of at least one ion channel selected from the group consisting of EAAT1 ion channel and NCX1 ion channel.

In one embodiment of any aspect provided herein, the method further comprises contacting with at least a second agent that alters the function of at least one ion channel selected from the group consisting of EAAT1 ion channel and NCX1 ion channel.

In one embodiment of any aspect provided herein, the agonist or agent and the at least one second agent are contacted at substantially the same time.

In one embodiment of any aspect provided herein, the agonist or agent and the at least one second agent are contacted at different times.

In one embodiment of any aspect provided herein, the at least one second agent is an inhibitor of the NCX1 ion channel.

In one embodiment of any aspect provided herein, the inhibitor is KB-R7943 mesylate.

In one embodiment of any aspect provided herein, the agent or agonist is comprised in a vehicle that is or comprises deuterium oxide.

In one embodiment of any aspect provided herein, the contacting is short-term. In one embodiment of any aspect provided herein, the contacting is long-term. In one embodiment of any aspect provided herein, the contacting is a single contact. In one embodiment of any aspect provided herein, the contacting comprises reoccurring contacting.

In one embodiment of any aspect provided herein, one or more genes listed in Table 1, or a gene product thereof, are modulated by the agent or agonist following contacting.

In one embodiment of any aspect provided herein, the contacting is performed for at least 1 hour, 2 hours, 3 hours, 4 hours, 5 hours, 6 hours, 10 hours, 18 hours, 24 hours, 36 hours 48, hour, 96 hours or more.

In one embodiment of any aspect provided herein, contacting is performed via diffusion, perfusion, injection, immersion, or delivery via air.

In one embodiment of any aspect provided herein, the diffusion, perfusion, injection, immersion, or delivery via air is performed in vivo or ex vivo.

In one embodiment of any aspect provided herein, contacting is performed via direct introduction to the cell, tissue or organ.

In one embodiment of any aspect provided herein, the cell, tissue or organ is contacted prior to removal from the donor for a transplant in a recipient.

In one embodiment of any aspect provided herein, the cell, tissue or organ is preserved contacted following removal from the donor, and prior to a transplant in a recipient.

In one embodiment of any aspect provided herein, the cell, tissue or organ is contacted following an injury to the cell, tissue or organ. In one embodiment of any aspect provided herein, the cell, tissue or organ is contacted prior to a surgical procedure. In one embodiment of any aspect provided herein, the cell, tissue or organ is contacted during a therapeutic treatment.

In one embodiment of any aspect provided herein, the therapeutic treatment is an anti-cancer treatment. Exemplary anti-cancer treatments include radiation, chemotherapy, immunotherapy, CAR-T cell therapy, cellular therapy, or engineered tissue constructs.

In one embodiment of any aspect provided herein, the contacting permits treatment with a higher dose of anti-cancer treatment relative to treatment in the absence of the contacting.

In one embodiment of any aspect provided herein, the method further comprises contacting the cell, tissue or organ with at least a second, biostatic compound. In one embodiment of any aspect provided herein, the at least a second compound is selected from the group consisting of hydrogen sulfide, nitrogen, argon, Oligomycin A, rotenone, 2-deoxyglucose, adenosine monophosphate (AMP), a neuropeptide, deferoxamine, and a prolyl hydroxylase inhibitor.

In one embodiment of any aspect provided herein, the cell, tissue or organ are contacted with the agonist and the at least a second compound at substantially the same time. In one embodiment of any aspect provided herein, the cell, tissue or organ are contacted with the agonist and the at least a second compound at different times.

In one embodiment of any aspect provided herein, the cell, tissue or organ is contacted with the agonist under a condition selected from the group consisting of hypoxia, osmotic stress, physiological stress, burn injury, blast injury, trauma, radiation, chemical exposure, toxin exposure and cooling or freezing condition.

In one embodiment of any aspect provided herein, the contacting comprises induction of biostasis that is reversed following withdrawal of the agonist and/or administration of an opioid antagonist.

In one embodiment of any aspect provided herein, the contacting does not induce hypothermia.

In one embodiment of any aspect provided herein, the agonist is not contacted in combination with a local anesthetic, an anti-arrhythmic, citrate, or magnesium.

Another aspect described herein provides a method of inducing biostasis in a cell, tissue or organ, the method comprising contacting the cell, tissue or organ in need of preservation with at least two agents that alter the function of at least one ion channel selected from the group consisting of EAAT1 ion channel and NCX1 ion channel, wherein the contacted cell, tissue or organ exhibits biostasis.

Another aspect described herein provides a method of preserving viability or function of a cell, tissue or organ, the method comprising contacting the cell, tissue or organ in need of such preservation with at least two agents that alter the function of at least one ion channel selected from the group consisting of EAAT1 ion channel and NCX1 ion channel.

Another aspect described herein provides a method of cell, tissue or organ transplant, the method comprising contacting a donor cell, tissue or organ in situ or ex vivo with at least two agents that alter the function of at least one ion channel selected from the group consisting of EAAT1 ion channel and NCX1 ion channel.

Another aspect described herein provides a method of preparing a cell, tissue or organ for transplant, the method comprising contacting a donor cell, tissue or organ with at least two agents that alter the function of at least one ion channel selected from the group consisting of EAAT1 ion channel and NCX1 ion channel.

Another aspect described herein provides a method of inducing biostasis in a cell, tissue or organ, the method comprising contacting the cell, tissue or organ in need of preservation with an agonist for the δ-opioid receptor and an agent that alters the function of at least one ion channel selected from the group consisting of EAAT1 ion channel and NCX1 ion channel, wherein the contacted cell, tissue or organ exhibits biostasis, wherein the agonist is administered at a dose sufficient to alter the function of at least one ion channel selected from the group consisting of EAAT1 ion channel and NCX1 ion channel.

Another aspect described herein provides a method of preserving viability or function of a cell, tissue or organ, the method comprising contacting the cell, tissue or organ in need of such preservation with an agonist of the δ-opioid receptor and an agent that alters the function of at least one ion channel selected from the group consisting of EAAT1 ion channel and NCX1 ion channel, wherein the agonist is administered at a dose sufficient to alter the function of at least one ion channel selected from the group consisting of EAAT1 ion channel and NCX1 ion channel.

Another aspect described herein provides a method of cell, tissue or organ transplant, the method comprising contacting a donor cell, tissue or organ in situ or ex vivo with an agonist of the δ-opioid receptor and an agent that alters the function of at least one ion channel selected from the group consisting of EAAT1 ion channel and NCX1 ion channel, wherein the agonist is administered at a dose sufficient to alter the function of at least one ion channel selected from the group consisting of EAAT1 ion channel and NCX1 ion channel.

Another aspect described herein provides a method of preparing a cell, tissue or organ for transplant, the method comprising contacting a donor cell, tissue or organ with an agonist of the δ-opioid receptor and an agent that alters the function of at least one ion channel selected from the group consisting of EAAT1 ion channel and NCX1 ion channel, wherein the agonist is administered at a dose sufficient to alter the function of at least one ion channel selected from the group consisting of EAAT1 ion channel and NCX1 ion channel.

In one embodiment of any aspect provided herein, at least one of the at least two agents is/are contacted at a sub-biostasis dose (i.e., a dose below the dose threshold required to induce biostasis as a single biostatic agent).

In one embodiment of any aspect provided herein, the agonists or agents are contacted at a sub-biostasis dose.

Another aspect described herein provides a composition comprising at least two agents that alter the function of at least one ion channel selected from the group consisting of EAAT1 ion channel and NCX1 ion channel.

Another aspect described herein provides a composition comprising an agonist of the δ-opioid receptor and an agent that alters the function of at least one ion channel selected from the group consisting of EAAT1 ion channel and NCX1 ion channel.

In one embodiment of any aspect provided herein, the composition further comprises deuterium oxide.

Another aspect described herein provides a composition comprising deuterium oxide and an agent that alters the function of at least one ion channel selected from the group consisting of EAAT1 ion channel and NCX1 ion channel.

Another aspect described herein provides a composition comprising deuterium oxide and an agonist of the δ-opioid receptor.

Another aspect described herein provides a method of preserving healthy cells in a subject undergoing a cancer treatment, the method comprising administering to the subject receiving or to receive an anti-cancer therapy (a) an agonist of the δ-opioid receptor, wherein the agonist is administered at a dose sufficient to alter the function of at least one ion channel selected from the group consisting of EAAT1 ion channel and NCX1 ion channel; or (b) an agent that alters the function of at least one ion channel selected from the group consisting of EAAT1 ion channel and NCX1 ion channel.

In one embodiment of any aspect provided herein, the administering is performed prior to, at substantially the same time, and/or after receiving an anti-cancer therapy.

In one embodiment of any aspect provided herein, the anti-cancer treatment is high dose or high exposure treatment.

In one embodiment of any aspect provided herein, administering is systemic or local administration. In one embodiment of any aspect provided herein, local administration is perfusion.

In one embodiment of any aspect provided herein, the agonist prevents or reduces cell death of non-cancer cells during the anti-cancer treatment. Another aspect described herein provides a method of treating a hematological neoplastic disease, the method comprising harvesting bone marrow from a subject having such a disease, contacting harvested bone marrow or a cellular fraction thereof with (a) an agonist of the δ-opioid receptor, wherein the agonist is administered at a dose sufficient to alter the function of at least one ion channel selected from the group consisting of EAAT1 ion channel and NCX1 ion channel; or (b) an agent that alters the function of at least one ion channel selected from the group consisting of EAAT1 ion channel and NCX1 ion channel; and with one or more anti-cancer therapeutics at a dose sufficient to kill neoplastic cells, treating the subject with chemotherapy or radiation sufficient to kill remaining bone marrow hematologic stem cells, and then administering the contacted bone marrow or cellular fraction to the subject.

In one embodiment of any aspect provided herein, the treatment with the agonist or agent protects non-neoplastic cells from killing by the one or more anti-cancer therapeutics.

In one embodiment of any aspect provided herein, the cell, tissue or organ is of human origin.

In one embodiment of any aspect provided herein, the cell, tissue or organ is of non-human origin.

Another aspect described herein provides a composition comprising a live explanted cell, tissue or organ in contact with a δ-opioid receptor agonist, wherein the agonist is present in an amount sufficient to alter the function of at least one ion channel selected from the group consisting of EAAT1 ion channel and NCX1 ion channel; or an agent that alters the function of at least one ion channel selected from the group consisting of EAAT1 ion channel and NCX1 ion channel.

In one embodiment of any aspect provided herein, the composition does not further comprise local anesthetic, an anti-arrhythmic, citrate, or magnesium.

In one embodiment of any aspect provided herein, the composition further comprises at least a second agent that alters the function of at least one ion channel selected from the group consisting of EAAT1 ion channel and NCX1 ion channel

In one embodiment of any aspect provided herein, the composition further comprises deuterium oxide. One aspect described herein provides a method of inducing biostasis in a cell, tissue or organ, the method comprising contacting the cell, tissue or organ in need of preservation with SNC-80, wherein the contacted cell, tissue or organ exhibits biostasis.

In one embodiment of any aspect provided herein, the agonist or agent is administered at or about 100 μM.

One aspect described herein provides a method of preserving viability or function of a cell, tissue or organ, the method comprising contacting the cell, tissue or organ in need of such preservation with SNC-80.

One aspect described herein provides a method of cell, tissue or organ transplant, the method comprising contacting a donor cell, tissue or organ in situ or ex vivo with SNC-80.

One aspect described herein provides a method of preparing a cell, tissue or organ for transplant, the method comprising contacting a donor cell, tissue or organ with SNC-80.

One aspect described herein provides a method of preserving healthy cells in a subject undergoing a cancer treatment, the method comprising administering to the subject receiving or to receive an anti-cancer therapy SNC-80.

One aspect described herein provides a method of treating a hematological neoplastic disease, the method comprising harvesting bone marrow from a subject having such a disease, contacting harvested bone marrow or a cellular fraction thereof with SNC-80 and with one or more anti-cancer therapeutics at a dose sufficient to kill neoplastic cells, treating the subject with chemotherapy or radiation sufficient to kill remaining bone marrow hematologic stem cells, and then administering the contacted bone marrow or cellular fraction to the subject.

One aspect described herein provides a composition comprising a live explanted cell, tissue or organ in contact with SNC-80.

One aspect described herein provides a method of preserving viability or function of a cell, tissue or organ, the method comprising contacting the cell, tissue or organ in need of such preservation with SNC-80.

One aspect described herein provides a method of cell, tissue or organ transplant, the method comprising contacting a donor cell, tissue or organ in situ or ex vivo with SNC-80.

One aspect described herein provides a method of preparing a cell, tissue or organ for transplant, the method comprising contacting a donor cell, tissue or organ with SNC-80.

One aspect described herein provides a method of inducing biostasis in a cell, tissue or organ, the method comprising contacting the cell, tissue or organ in need of preservation with Donepezil, wherein the contacted cell, tissue or organ exhibits biostasis.

One aspect described herein provides a method of preserving viability or function of a cell, tissue or organ, the method comprising contacting the cell, tissue or organ in need of such preservation with Donepezil.

One aspect described herein provides a method of cell, tissue or organ transplant, the method comprising contacting a donor cell, tissue or organ in situ or ex vivo with Donepezil.

One aspect described herein provides a method of preparing a cell, tissue or organ for transplant, the method comprising contacting a donor cell, tissue or organ with Donepezil.

One aspect described herein provides a method of preserving healthy cells in a subject undergoing a cancer treatment, the method comprising administering to the subject receiving or to receive an anti-cancer therapy Donepezil.

One aspect described herein provides a method of treating a hematological neoplastic disease, the method comprising harvesting bone marrow from a subject having such a disease, contacting harvested bone marrow or a cellular fraction thereof with Donepezil and with one or more anti-cancer therapeutics at a dose sufficient to kill neoplastic cells, treating the subject with chemotherapy or radiation sufficient to kill remaining bone marrow hematologic stem cells, and then administering the contacted bone marrow or cellular fraction to the subject.

One aspect described herein provides a composition comprising a live explanted cell, tissue or organ in contact with Donepezil.

One aspect described herein provides a method of preserving viability or function of a cell, tissue or organ, the method comprising contacting the cell, tissue or organ in need of such preservation with Donepezil.

One aspect described herein provides a method of cell, tissue or organ transplant, the method comprising contacting a donor cell, tissue or organ in situ or ex vivo with Donepezil.

One aspect described herein provides a method of preparing a cell, tissue or organ for transplant, the method comprising contacting a donor cell, tissue or organ with Donepezil.

One aspect described herein provides a method of slowing viral replication or a viral infection in a subject, the method comprising administering SNC-80 to a subject in need thereof.

One aspect described herein provides a method of slowing viral replication or a viral infection in a subject, the method comprising administering Donepezil to a subject in need thereof.

In one embodiment of any aspect herein, the subject has or is at risk of having a viral infection.

In one embodiment of any aspect herein, the administration is local or systemic.

One aspect described herein provides a method of slowing viral replication or a viral infection in an organ or tissue, the method comprising contacting the organ or tissue with SNC-80.

One aspect described herein provides a method of slowing viral replication or a viral infection in an organ or tissue, the method comprising contacting the organ or tissue with Donepezil.

Without wishing to be bound by theory, it is contemplated that a given agent, e.g., SNC-80 or Donepezil, among others, can act in different ways during the process of inducing biostasis. For example, as the concentration of the drug in the tissue, organ or organism increases, those pathways most sensitive to the drug will be affected first, followed by pathways that are only sensitive at higher dosages, providing a sequential saturation of the various pathways, ultimately resulting in biostasis. In this manner, then, without wishing to be bound by theory, it is contemplated that a range or continuum of mechanisms may be involved in the process of inducing and/or reversing biostasis with an agent as described herein.

One aspect described herein provides a method of restoring metabolic activity in a cell, tissue or organ that has been contacted by an agonist of the δ-opioid receptor, wherein the agonist is administered at a dose sufficient to alter the function of at least one ion channel selected from the group consisting of EAAT1 ion channel and NCX1 ion channel or an agent that alters the function of at least one ion channel selected from the group consisting of EAAT1 ion channel and NCX1 ion channel, the method comprising contacting the cell, tissue or organ with a polyol. Preferred polyols can modulate intra- or inter-molecular motion through interaction with the hydrogen shells of proteins.

One aspect described herein provides a method of restoring normal metabolic function in a cell, tissue or organ that has been contacted by an agonist of the δ-opioid receptor, wherein the agonist is administered at a dose sufficient to alter the function of at least one ion channel selected from the group consisting of EAAT1 ion channel and NCX1 ion channel or an agent that alters the function of at least one ion channel selected from the group consisting of EAAT1 ion channel and NCX1 ion channel, the method comprising contacting the cell, tissue or organ with a polyol.

One aspect described herein provides a method of restoring oxidative metabolism in a cell, tissue or organ that has been contacted by an agonist of the δ-opioid receptor, wherein the agonist is administered at a dose sufficient to alter the function of at least one ion channel selected from the group consisting of EAAT1 ion channel and NCX1 ion channel or an agent that alters the function of at least one ion channel selected from the group consisting of EAAT1 ion channel and NCX1 ion channel, the method comprising contacting the cell, tissue or organ with a polyol.

One aspect described herein provides a method of restoring metabolic function is recovering a cell, tissue or organ that has been contacted by an agonist of the δ-opioid receptor to induce biostasis, wherein the agonist is administered at a dose sufficient to alter the function of at least one ion channel selected from the group consisting of EAAT1 ion channel and NCX1 ion channel or an agent that alters the function of at least one ion channel selected from the group consisting of EAAT1 ion channel and NCX1 ion channel, the method comprising contacting the cell, tissue or organ with a polyol.

In one embodiment of any aspect herein, the method further comprises the step, prior to contact with the polyol, of removing the agonist or agent from the organ or tissue.

In one embodiment of any aspect herein, the polyol is kestose or erlose.

### Definitions

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology and molecular biology can be found in The Merck Manual of Diagnosis and Therapy, 19th Edition, published by Merck Sharp & Dohme Corp., 2011 (ISBN 978-0-911910-19-3); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), Taylor & Francis Limited, 2014 (ISBN 0815345305, 9780815345305); Lewin's Genes XI, published by Jones & Bartlett Publishers, 2014 (ISBN-1449659055); Michael Richard Green and Joseph Sambrook, Molecular Cloning: A Laboratory Manual, 4^{th} ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

The terms “decrease”, “reduced”, “reduction”, or “inhibit” are all used herein to mean a decrease by a statistically significant amount. In some embodiments of any of the aspects, “reduce,” “reduction” or “decrease” or “inhibit” typically means a decrease by at least 10% as compared to a reference level (e.g. the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase”, “enhance”, or “activate” are all used herein to mean an increase by a statically significant amount. In some embodiments of any of the aspects, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, a “increase” is a statistically significant increase in such level.

As used herein, a “subject” means a human or animal. Usually the animal is a vertebrate such as a primate, rodent, domestic animal or game animal. Primates include chimpanzees, cynomolgus monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments of any of the aspects, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of conditions described herein. A subject can be male or female.

A subject can be one who has been previously diagnosed with or identified as suffering from or having a condition in need of treatment, e.g., a cancer treatment, a traumatic wound or a cell, tissue or organ transplantation, or one or more complications related to such a condition, and optionally, have already undergone treatment for the condition or the one or more complications related to the condition. Alternatively, a subject can also be one who has not been previously diagnosed as having the condition or one or more complications related to the condition. For example, a subject can be one who exhibits one or more risk factors for the condition or one or more complications related to the condition or a subject who does not exhibit risk factors.

As used herein, “biostasis” refers to a state of a biological system in which metabolism is slowed and energy demands reduced such that a cell, tissue, organ or a whole organism remains viable but respiration, biochemical processes and metabolic demands are reduced such that the system maintains viability under conditions that, absent the induction of the biostatic state, would normally kill the cell, tissue, organ or organism. As the term is used herein, biostasis is reversible, such that the cell, tissue, organ or organism returns to substantially normal metabolic and physical activity upon withdrawal of an inducer or inducers of biostasis.

As used herein, “preserving” refers to maintaining the original physiological state and functionality of a cell, tissue or organ prior to or upon removal from a donor for transplant. In one embodiment, preserving is achieved by treatment of the donor or donor cell, tissue or organ prior to removal from a donor.

As used herein, “organ failure” refers to a change in organ function in critically ill patients that require medical intervention to achieve homeostasis.

As used herein, “agonist” refers to a compound that binds to and activates signaling by a receptor, causing a response in a cell.

An “organ transplant” refers to transferring or “transplanting” an internal organ (for example, heart, lung, kidney, liver, pancreas, stomach, large intestine and small intestine and bone marrow) or external organ (for example, skin, cornea) from a donor subject to a recipient subject. In one embodiment, an organ transplant is from one subject to another, usually genetically distinct, subject. In another embodiment, a transplant is from a given subject, back to that subject, e.g., as is the case in autologous stem cell transplant. An “organ transplant” also includes cross-species transplants (e.g., xenotransplants).

As used herein, “modulate,” “modulates,” “modulation” or variations on these terms refer to an increase or a decrease in a given parameter or activity as those terms are defined herein.

A “subject in need” of treatment e.g., a cancer treatment or a cell, tissue or organ transplant, for a particular condition can be a subject having that condition, diagnosed as having that condition, or at risk of developing that condition.

As used herein, the terms “treat,” “treatment,” or “treating,” refer to therapeutic treatments, wherein the object is to reverse, alleviate, ameliorate, inhibit, slow down or stop the progression or severity of a condition associated with a disease or disorder, e.g., cancer, trauma or a disease that results in the need for a cell, tissue or organ transplant. The term “treating” includes reducing or alleviating at least one adverse effect or symptom of a condition, disease or disorder associated with a condition described herein. Treatment is generally “effective” if one or more symptoms or clinical markers of a disease, disorder or condition are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

As used herein, the term “pharmaceutical composition” refers to an active agent in combination with a pharmaceutically acceptable carrier, e.g., a carrier commonly used in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. In some embodiments of any of the aspects, a pharmaceutically acceptable carrier can be a carrier other than water. In some embodiments of any of the aspects, a pharmaceutically acceptable carrier can be a cream, emulsion, gel, liposome, nanoparticle, and/or ointment. In some embodiments of any of the aspects, a pharmaceutically acceptable carrier can be an artificial or engineered carrier, e.g., a carrier that the active ingredient would not be found to occur in in nature.

As used herein, the term “contacting,” refers to the placement or introduction of, for example, an agonist or other agent as disclosed herein, on or into, e.g., a cell, tissue, organ, or subject by a method or route which results in at least partial delivery of the agent at a desired site. Contacting can be in vivo, ex vivo, or in situ. Preferably, contacting is ex vivo or in situ. Exemplary methods of contacting include perfusion or immersion of a cell, tissue, organ, or subject with the agonist or other agent as described herein. Preferably, the agonist or other agent is directly introduced to the cell, tissue, or organ.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean±1%.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

### BRIEF DESCRIPTION OF THE DRAWINGS

This application file contains at least one drawing executed in color. Copies of this patent application publication with color drawings will be provided by the Office upon request and payment of the necessary fee.

FIG. 1 shows SNC-80 induces hypothermia in mice and rats and can be modulated through dose and drug combinations (e.g., fluoxetine). Data presented in, e.g., Rawls and Cowan 2006.

FIG. 2 shows tail length assay at 24 h of drug exposure as metric for Xenopus development. Developed tail length assay are used to assess embryo tail development during drug contact. Compounds marked with (*) showed the significant arrest in development.

FIG. 3 shows network profile comparison of SNC80 tadpoles with early torpor in ground squirrels. Differences are concentrated around key nodes along the network spine. Data presented herein indicate torpor-like state and SNC-80 both impact network control structure. SNC-80 treatment of Xenopus is similar to aggregate Arctic ground squirrel torpor datasets (˜50% similar gene expression patterns).

FIG. 4 shows SNC80 in Caco-2 cells reduces intracellular ATP while leaving respiration unchanged.

FIG. 5 shows NAD/NADH live reporting in Coco-2 cells treated with SNC-80. 100 uM of SNC80 results in a significant increase (****, p<0.0005) in NAD/NADH ratio comparable to the positive control sodium oxamate.

FIG. 6 shows continuous Xenopus embryo development tracking reveals transient SNC-80 effects. Automated embryo culture and imaging platform with machine vision processing. Analysis pipeline allows screening scale-up to hundreds of embryos at a given time. 0.4 μM SNC-80 (n=5) resulted in a 19.8% reduction of embryo length compared to control (n=6) at the end of the dosing period, t=48 h. Delayed embryos caught up to controls after drug were removed.

FIG. 7 shows SNC-80 reduces oxygen consumption of embryos.

FIG. 8 shows SNC80 demonstrates reversible biostatic effect in Xenopus laevis tadpoles.

FIG. 9 shows Xenodose Platform: Time-Dependent Drug Delivery for Time-Modulating Interventions. Dynamic drug dosing platform comprises a pumping system, flowcells or vials that contain the specific targeted organisms, sensors that measure specific parameters and provides feedback to the pumping system, and enables assessment of drug PKPD and implementation of control theory to pharmacology.

FIG. 10 shows initial exploration of dynamic dosing of SNC-80 in tadpoles. Dynamic drug dosing used to examine stasis control parameters (10 tadpoles/chamber, n=3 replicates). SNC-80 (60 μM) dose shows ˜10 min delay in impacting oxygen metabolism.

FIG. 11 shows exemplary discovered network nodes with ability to exert control over communities. Spine like structure detected with approximately 6 dense communities attached to it. Key control genes on the spine include EGR1 (mediates hypoxia response), GLI2 (regulates of biomacromolecule synthesis), ONECUT2 (promotes cell differentiation and cell fate), PHOX2B (regulates parasympathetic nervous system development), TWIST1 (promotes cellular response to decreased oxygen levels), and TWIST2 (controls negative regulation of transcription). Further, 52 genes that sit one step off the spine are up- or down-regulated during early torpor. It is possible to control/perturb communities via their connection to spine to replicate a state network profile.

FIG. 12 shows development of a new pipeline for assessing dynamic networks. This new pipeline can be used to assess gene network perturbations in response to candidate biostasis drugs. The steps of the pipeline include (1) Sub-sample the gene network at each time point, (2) Assess network structure, key motifs, and clustering, (3) Use gene ontology to identify and validate pathway dynamics. It is noted that all pathways should share dynamics. Gene ontology analysis of all genes above a threshold revealed a strong connection to hypoxia response with an average path distance of 4.04 to HIF1A. Some of the top ranked GO terms include, Positive regulation of histone H3-K27 methylation, Response to hypoxia, Chromatin silencing at telomerem Negative regulation of glutamate secretions, and Pyroptosis.

FIGS. 13A and 13B show effects of WC-22 (Donepezil) treatment. FIG. 13A shows that Xenopus tadpoles treated with WC-22 (Donepezil) have reduced tail length as compared to a vehicle control or SNC-80, indicating that Donepezil slightly reduced growth in Xenopus. FIG. 13B shows that Donepezil treatment markedly reduced oxygen consumption in Xenopus tadpoles, as compared to a vehicle control. This effect is not due to lack of motion alone; Donepezil treatment markedly reduced oxygen consumption in Xenopus tadpoles, as compared to treatment with 1×Tricaine, an anesthetic control.

FIG. 14 shows rapid slowing of motion following Donepezil treatment in Xenopus tadpoles. This slowing of motion is reversible; average movement of the Xenopus tadpoles is returned to pre-treatment levels following removal of Donepezil. Slowing occurred within 10 min following 50 uM Donepezil addition. The drug is removed at 30 minutes, as indicated by the hash-line. Movement resumed within 50 min after drug removal.

FIG. 15 shows that Donepezil stasis, or reversal does not alter cognitive or motor function performance of Xenopus. Tadpoles were exposed to 25 uM or 50 uM Donepezil for 30 min, to induce stasis, and allowed to recover. Tadpoles were then tested over a 24 h period in an automated behavior and cognitive testing platform to measure cognitive or motor function. Donepezil-treated tadpoles did not exhibit decreased cognitive or motor function, as compared to control tadpoles. 50 uM condition may have slightly improved cognitive performance.

FIG. 16 shows oxygen uptake in pig hind limbs treated with SNC-80, or control-treated. Treatment with SNC-80 markedly reduces oxygen uptake in the hind limb as compared to control treated.

FIG. 17 shows relative metabolic rate in pig hind limbs treated with SNC-80 or control-treated with perfusion medium alone. Treatment with SNC-80 markedly reduces metabolic rate in the hind limb as compared to control treated.

FIG. 18 shows relative metabolic rate in pig hind limbs treated 3 hours post SNC-80 or control-treatment. A marked reduction of metabolic rate is observed 3 hours post treatment with SNC-80 as compared to control treated. SNC-80 slope is significantly different from Control (P<0.0001), 95% CI shown.

FIG. 19 shows relative metabolic rate by phases post SNC-80 or control-treatment. Treatment and Washout phases are statistically significant between SNC-80 and control treatment.

FIG. 20 shows the change in mass of limb prior to and following treatment. SNC-80 results in a larger change in a mass as compared to a control treated limb.

FIG. 21 shows histology of limb muscle prior to and following treatment with SNC-80. An increase in intercellular edema that corresponds to weight increase observed in limbs. Muscle bundle area did not increase significantly, which means that the muscle remains intact during the preservation period

FIG. 22 shows histology of glycogen content prior to and following treatment with SNC-80. Glycogen content is visualized via PAS immunofluorescence. Analysis of optical density to calculate glycogen content revealed that glycogen content inside the cells remains at a basal level

FIG. 23 shows potassium, lactate, and glucose levels in pig hind limbs treated with SNC-80, or control-treated. No change in potassium, lactate, or glucose levels is observed following SNC-80 treatment as compared to control treatment.

FIG. 24 shows a schematic of ion exchange of the EAAT and NCX1 channels.

FIGS. 25A-25B show oxygen consumption in tadpoles contacted by the indicated compounds. (FIG. 25A) No change in oxygen consumption was observed following contact with 25 μM SNC-80 (WC1). (FIG. 25B) No change in oxygen consumption was observed following contact with 35 μM KB-R7943 Mesylate (WC60). (FIG. 25C) A reduction in oxygen consumption was observed following contact with 25 μM SNC-80 and 35 μM KB-R7943 Mesylate, indicating an additive/synergistic effect.

FIGS. 26A-26B show recovery from Donepezil (WC-22)-induced biostasis in tadpoles contacted by the indicated compounds. (FIG. 26A) Increased tail length was observed in tadpoles that recovered in kestose (WC33) as compared to control treatment. (FIG. 26B) An increased rate of oxygen consumption was observed in tadpoles that recovered in kestose (WC33) as compared to MMR alone.

FIG. 27 shows embryonic tail length of Xenopus embryos contacted with various concentrations of deuterium oxide (²H²O). Statistical tests performed: 2-way ANOVA with multiple comparisons N=5/group.

FIG. 28 shows recovery of Xenopus embryos from 4 days of biostasis induced by various concentrations of deuterium oxide (²H₂O). Statistical tests performed: 1-way ANOVA with pairwise comparisons N=5/group.

FIG. 29 shows 50% ²H₂O-treated tadpoles exhibit longer life span in low-oxygen environments. At the indicated consumption rate, ²H₂O-treated tadpoles survive for >30 hours as compared to 6 hours for MMR controls.

FIG. 30 show 50% ²H₂O-treated embryos have reduced oxygen consumption within hours of contact.

FIG. 31 shows embryonic tail length of Xenopus embryos contacted with 50% ²H₂O at 24 hours and 48 hours.

FIG. 32 shows free swimming tadpoles exposed to WC61 (Aprindine) at 25 uM results in decreased oxygen consumption after 20 min of exposure as compared to vehicle controls.

FIG. 33 shows that delta opioid receptor antagonist Naltrindole does not block the stasis-inducing effects of WC1 (SNC-80) at concentrations up to 100 uM during 4 h of exposure. Oxygen consumption in tadpoles remains decreased when exposed to a stasis inducing concentration of SNC-80 in combination with 100 uM Naltrindole. These data indicate that activation of the opioid pathway may not be required for SNC-80-induced stasis.

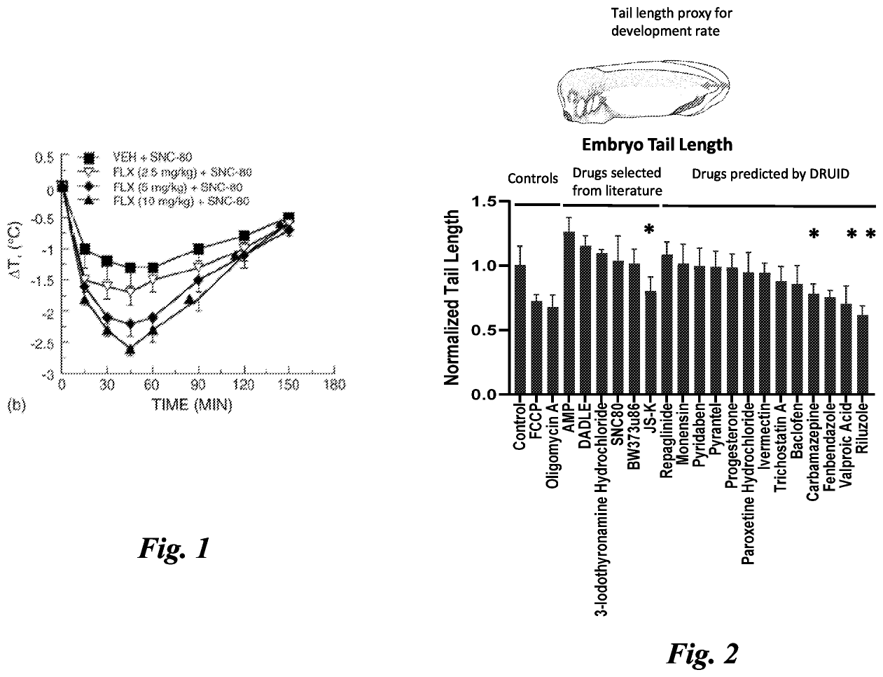

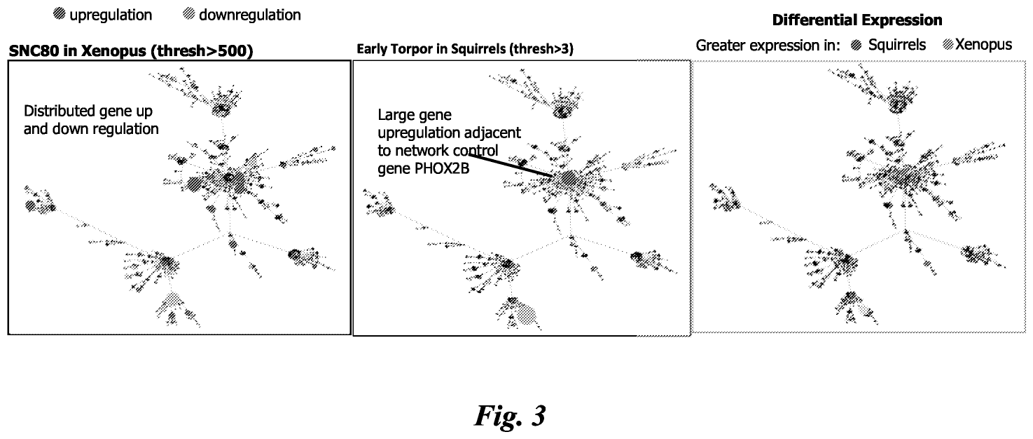

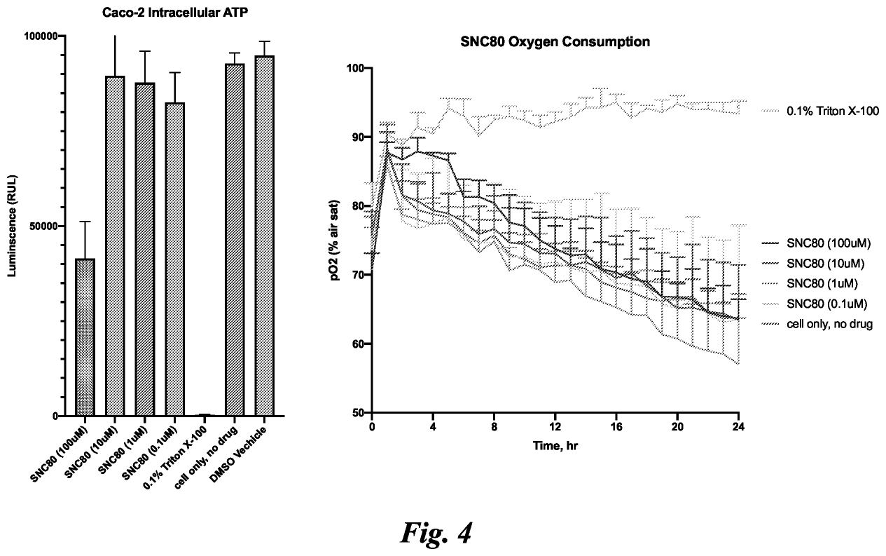

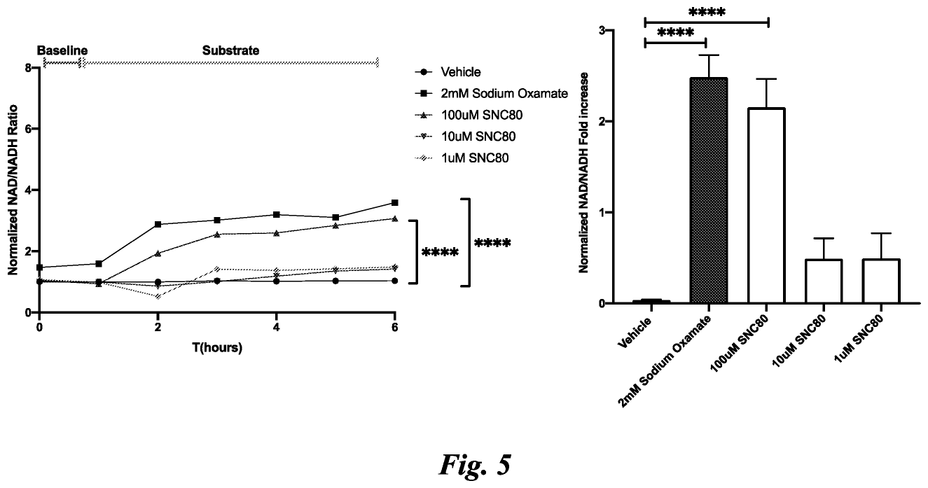

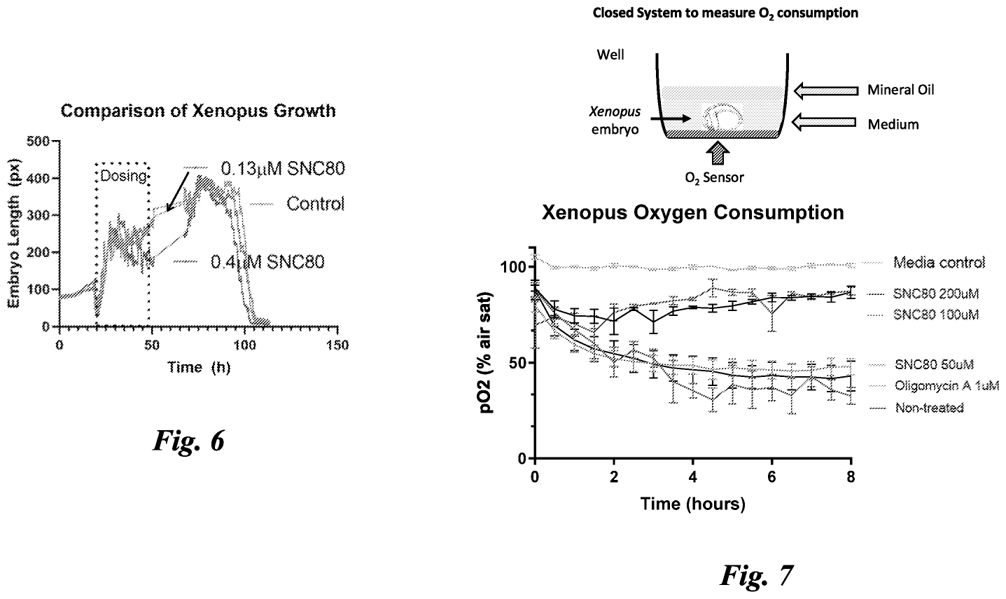

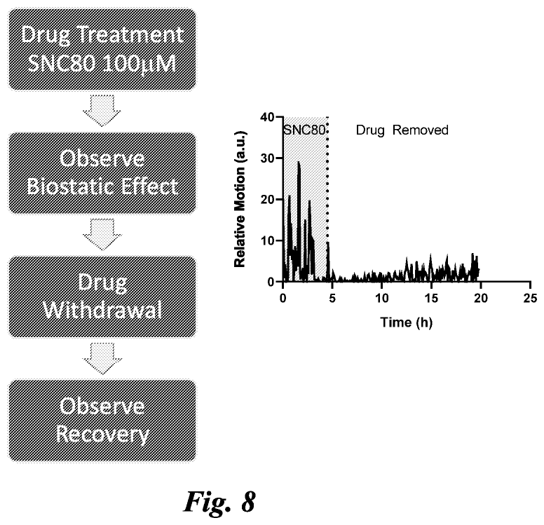

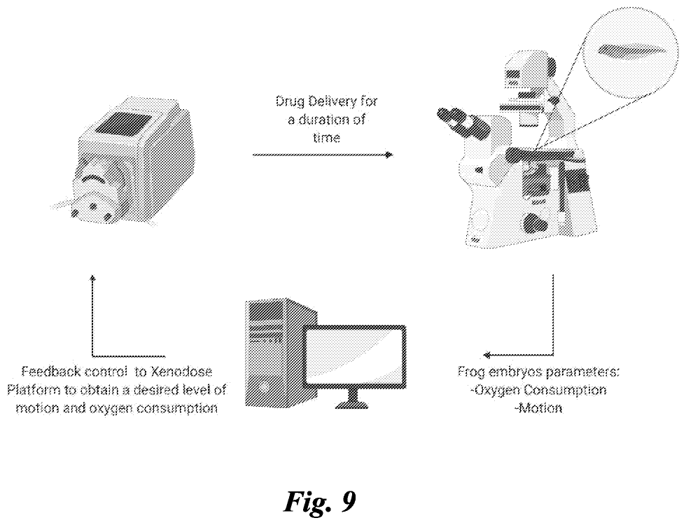

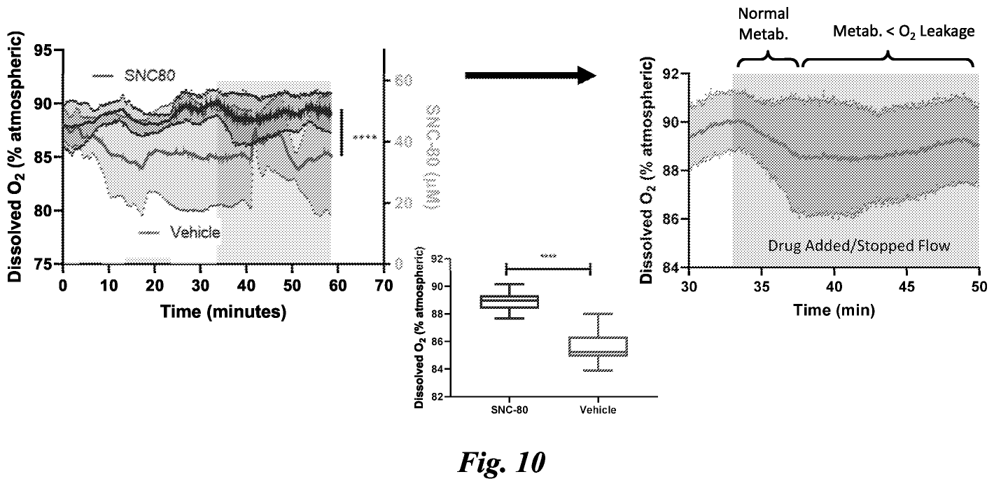

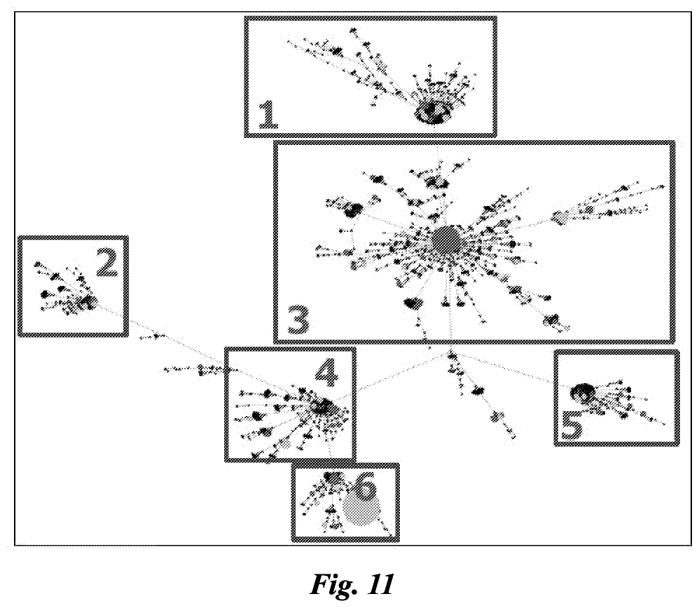

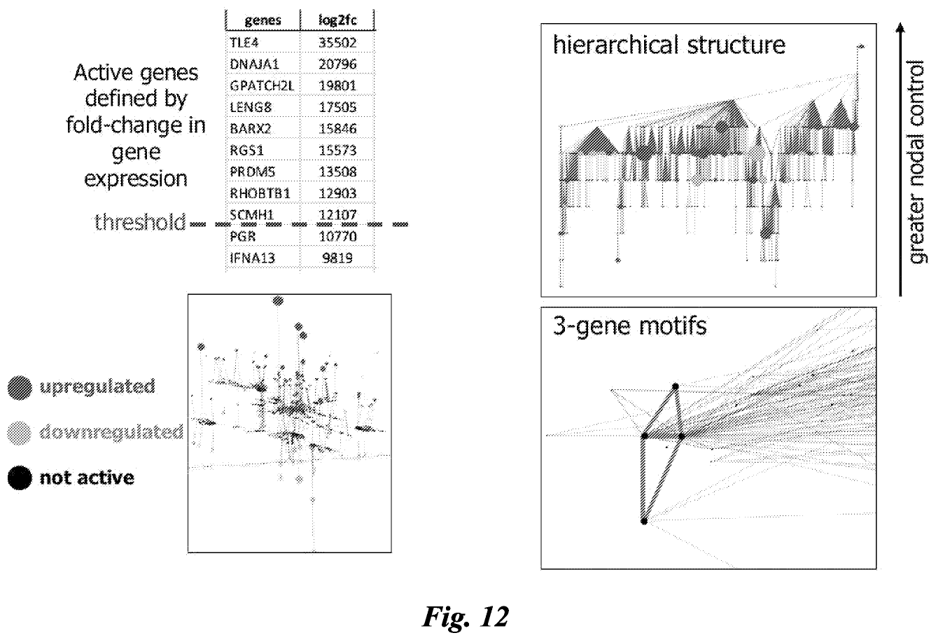

### DETAILED DESCRIPTION

Opioid drugs are typically classified by their binding selectivity in respect of the cellular and differentiated tissue receptors to which a specific drug species binds as a ligand. At least three subtypes of opioid receptors (mu, delta and kappa) are described and documented in the scientific literature. All three receptors are present in the central and peripheral nervous systems of many species including human. Activation of delta receptors produces antinociception in rodents and can induce analgesia in man, in addition to influencing motility of the gastrointestinal tract. (See Burks, T. F. (1995) in “The Pharmacology of Opioid Peptides”, edited by Tseng, L. F., Harwood Academic Publishers).

Various aspects of the technology described herein comprise contacting a cell, tissue or organ with an agonist of the δ-opioid receptor.

As used herein, the δ-opioid receptor, also known as DOP; DOR; DOR1; OPRD; and OPRD1, refers to an inhibitory 7-transmembrane G-protein coupled receptor (GPCR) coupled to the G protein Gi/GO and has enkephalins as its endogenous ligands. δ-opioid receptor sequences are known for a number of species, e.g., human δ-opioid receptor (NCBI Gene ID: 4985) polypeptide (e.g., NCBI Ref Seq NP_000902.3) and mRNA (e.g., NCBI Ref Seq MM_1.MM_000911.4). δ-opioid receptor can refer to human δ-opioid receptor, including naturally occurring variants and alleles thereof. To be clear, the mu and kappa opioid receptors are not variants or alleles of the delta opioid receptor as the terms are used herein. δ-opioid receptor refers to the mammalian δ-opioid receptor of, e.g., mouse, rat, rabbit, dog, cat, cow, horse, pig, and the like.

### δ-Opioid Receptor Agonists

Exemplary peptide agonists of δ-opioid receptor include, but are not limited to Leu-enkephalin, Met-enkephalin, Deltorphins, DADLE, DSLET and DPDPE. Exemplary non-peptide agonists of δ-opioid receptor include, but are not limited to spiroindanyloxymorphone, N-Phenethyl-14-ethoxymetopon, ADL-5859, BU-48, SNC-80, BW373U86, DPI-221, DPI-287, DPI-3290, TAN-67, RWJ-394674, Desmethylclozapine, Norbuprenorphine (peripherally restricted), Cannabidiol (allosteric modulator, non-selective), Tetrahydrocannabinol (allosteric modulator, non-selective), orphanol, Mitragyna speciosa (kratom) indole derivatives (e.g., Mitragynine, and Mitragynine pseudoindoxyl). Additional δ-opioid receptor agonists are further described in, e.g., published U.S. Pat. App. No. 2004/0138220, which is incorporated herein by reference; see also U.S. Pat. No. 8,575,169, which is incorporated herein by reference, and particularly column 8, line 33 to column 14, line 3 therein.

In one embodiment, the agonist is a compound that binds to and is selective for activation of the δ-opioid receptor. A selective δ-opioid receptor agonist activates δ-opioid receptor at least 20× more potently than the same composition activates the κ or μ opioid receptors, and preferably at least 30×, 40×, 50×, 60×, 70×, 80×, 90×, 100× or more potently.

In one embodiment, the δ-opioid receptor agonist is SNC-80. SNC-80 is an opioid analgesic drug discovered in 1994 that selectively activates μ-δ opioid receptor heteromers and is used primarily in scientific research. SNC-80 was the first non-peptide drug developed that was regarded as a highly selective agonist for the δ-opioid receptor. It has been shown to produce useful analgesic, antidepressant and anxiolytic effects in animal studies, but its usefulness is limited as it produces convulsions at high doses. As such SNC-80 is not currently used as a medical therapeutic.

SNC-80, which has the chemical name, (+)-4-[(αR)-α-((2S,5R)-4-Allyl-2,5-dimethyl-1-piperazinyl)-3-methoxybenzyl]-N,N-diethylbenzamide, has the chemical structure shown below.

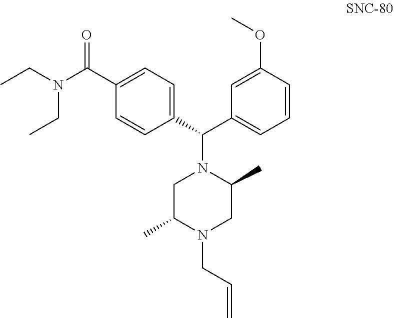 [SMILES: C=CCN1C[C@H](C)N([C@H](c2ccc(C(=O)N(CC)CC)cc2)c2cccc(OC)c2)C[C@H]1C]

In one embodiment, the agonist is an SNC-80 derivative, analog, or variant. In another embodiment, the SNC-80, derivative, analog or variant thereof, is formulated to take advantage of increased bioavailability of the delta-opioid receptor. Methods for formulating SNC-80 for increased receptor bioavailability are described in, e.g., U.S. Pat. No. 9,823,260B2, which is incorporated herein by reference in its entirety.

In various aspects herein, the agonist is administered at a dose sufficient to alter the function of at least one ion channel selected from the group consisting of EAAT1 ion channel and NCX1 ion channel. In one embodiment, the agonist is administered at a dose of 100 uM or in the range of doses of as described herein below. In one embodiment, the agonist is administered at a dose of 100 uM for at least 2 hours.

In one embodiment, the agonist is a derivative, analogue or variant of a known δ-opioid receptor agonist.

The term “derivative” as used herein refers to a chemical substance related structurally to another, i.e., related to an “original” substance, which can be referred to as a “parent” compound. A “derivative” can be made from the structurally-related parent compound in one or more steps. While specific properties and structure of a derivative may vary, the general physical and chemical properties of a derivative are generally similar to the parent compound. A derivative of SNC-80 as described herein will selectively bind to and activate a delta opioid receptor and/or alter the function of the EAAT1 or NCX1 ion channels.

The terms “functional derivative” and “mimetic” are used interchangeably herein, and refer to compounds which possess a biological activity (in particular functional biological activity, e.g., receptor binding, and activation) that is substantially similar to the biological activity of the entity or molecule of which it is a functional derivative. The term functional derivative is intended to include variants, analogues or chemical derivatives of a molecule. In certain embodiments, functional derivatives and functional analogues of opioid receptor agonists, e.g., δ-opioid receptor agonists, can be assessed for their biological activity, whether δ-opioid receptor activity or EAAT1 or NCX1 ion channel activity using an assay as described herein below, where derivatives and analogues which selectively activate δ opioid receptors or alter EAAT1 or NCX1 ion channel activity are or would be considered functional derivatives or functional analogues of such delta opioid receptor agonists.

The term “substantially similar,” when used to define the biological activity of a derivative or analogue of a delta opioid receptor agonist as compared to the biological activity of its parent agonist, means that a particular derivative or analogue differs from the parent agonist in chemical structure, by one or more groups or elements, including substitutions, deletions, or additions of groups or elements, the net effect of which is to retain at least some of the agonist activity found in the reference agonist. Such biological activity as a delta opioid receptor agonist by a functional derivative or analogue can be assessed by one of ordinary skill in the art using assays well known in the art, for example, receptor binding assays described herein below.

### Donepezil

Donepezil (marketed as Aricept®) is member of the acetylcholinesterase inhibitor class of drugs. Current indications for Donepezil are for dementia of the Alzheimer's type. Data regarding Donepezil show it functions as a reversible acetylcholinesterase inhibitor. Administration of the drug increases acetylcholine concentrations, which in turn enhances cholinergic neurotransmission. In addition to its actions as an acetylcholinesterase inhibitor, Donepezil has been found to act as a potent agonist of the σ₁ receptor (Ki=14.6 nM). Donepezil has been shown to produce specific antiamnesic effects in animals mainly via this action.

Some noncholinergic mechanisms of action have also been proposed for Donepezil.

Donepezil is shown to upregulate the nicotinic receptors in the cortical neurons, adding to neuroprotective property. Donepezil inhibits voltage-activated sodium currents reversibly and delays rectifier potassium currents and fast transient potassium currents.

Donepezil, which has the chemical name, 2-((1-Benzylpiperidin-4-yl)methyl)-5,6-dimethoxy-2,3-dihydro-1H-inden-1-one, has the chemical structure shown below.

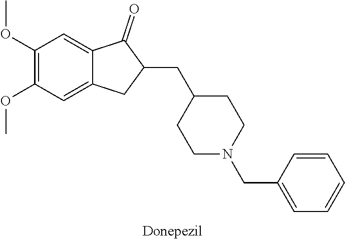 [SMILES: COc1cc2c(cc1OC)C(=O)C(CC1CCN(Cc3ccccc3)CC1)C2]

In one embodiment, an agent for inducing biostasis is a Donepezil derivative, analog, or variant. Such variants can be selected to have, for example, different solubility, stability or other properties relative to Donepezil, yet retain the ability to induce biostasis at, e.g., higher, lower or similar amounts relative to Donepezil.

Donepezil and Donepezil formulations are further described in, for example, International application Nos WO2006030249A1; WO2007129712A1; WO2006045512A1; and WO2008066179A1, and US Application Nos US20060183776A9; US20060280789A1; US20070129402A1; and US20060270709A1; the contents of which are incorporated herein by reference in their entireties.

Therapeutic dosages for Donepezil for treatment of Alzheimer's disease range from 5 mg-23 mg daily dependent upon the severity or progression of the disease. For example, for mild to moderate Alzheimer's disease, a subject is administered 5 mg daily for 4-6 weeks, and is then administered 10 mg daily. For moderate to severe Alzheimer's disease, a subject is administered 10 mg daily for at least 3 months, and is then administered 23 mg daily. Therapeutic doses are administered orally for Alzheimer's disease. The therapeutic range of 5 mg-23 mg Donepezil for treatment of Alzheimer's disease is not sufficient to alter the function of the EAAT1 and/or NCX1 ion channels.

In one embodiment, the dosage of Donepezil sufficient to induce biostasis is greater than the therapeutic range of 5 mg-23 mg Donepezil for treatment of Alzheimer's disease.

It is specifically contemplated herein that the dosage of Donepezil sufficient to induce biostasis is can be toxic to an individual, but that exposure to such a dosage will not have a toxic effect on an isolated cell, tissue or organ. In one embodiment, Donepezil is administered or contacted at 50 μM for at least 30 minutes.

### Receptor Binding and Activity Assays

Opioid (mu and kappa) receptor binding assays can be performed in guinea-pig brain membrane preparations, essentially as described by essentially as described by International Patent Application No. US20040138220, which is incorporated herein by reference in its entirety. Binding assays can be carried out at 25° C. for 60 minutes in 50 mM Tris (pH 7.4) buffer. [3H]-DAMGO(2 nM) and [3H]-U-69,593 (2 nM) can be used to label mu and kappa receptor binding sites, respectively. The protein concentration can be approximately 200 μg/well. Non-specific binding can be defined with 10 μM naloxone.

δ-receptor binding assays can be performed in a stable line of CHO cells expressing the human δ-receptor, essentially as described by International Patent Application No. US20040138220, which is incorporated herein by reference in its entirety. The binding assay can be carried out at 25° C. for 120 minutes in 50 mM Tris (pH 7.4) buffer. [3H]-SNC-80 can be used to label δ-receptor binding sites. The protein concentration can be approximately 12.5 μg/well. Non-specific binding can be defined with 10 μM naltrexone.

The binding reaction can be terminated by rapid filtration through glass fiber filters, and the samples can be washed with ice-cold 50 mM Tris buffer (pH 7.4).

Agonist activity at the delta, mu and kappa opioid receptors can be determined, for example, as follows.

Opioid (delta, mu and kappa) activity is commonly studied, as described below, in two isolated tissues, the mouse vas deferens (MVD)(δ) and the guinea-pig myentric plexus with attached longitudinal muscle (GPMP) (μ and k).

MVD (DC1 strain, Charles River, 25-35 g) are suspended in 15 ml organ baths containing Mg++ free Krebs' buffer of the following composition (mM): NaCl, 119; KCl, 4.7; NaHCO₃, 25; KH2PO4, 1,2; CaCl2, 2,5 and glucose, 11. The buffer is gassed with 95% O2 and 5% CO2. The tissues are suspended between platinum electrodes, attached to an isometric transducer with 500 mg tension and stimulated with 0.03 Hz pulses of 1-msec pulse-width at supramaximal voltage. IC50 values are determined by the regression analysis of concentration-response curves for inhibition of electrically-induced contractions in the presence of 300 nM of the mu-selective antagonist CTOP. This test is a measure of δ agonism.

Guinea-pig (Porcellus strain, male, 450-500 g, Dunkin Hartley) myentric plexus with attached longitudinal muscle segments are suspended with 1 g of tension in Krebs' buffer and stimulated with 0.1 Hz pulses of 1-msec pulse-width at supramaximal voltage. Mu functional activity is determined in the presence of 10 nM nor-BNI with 1 μM of the mu selective agonist, DAMGO, added to the bath at the end of the experiment to define a maximal response. This test is a measure of mu agonism.

Kappa functional activity is determined in the presence of 1 μM CTOP with 1 μM of the kappa selective agonist U-69,593 added at the end of the experiment to define a maximal response. All inhibitions of twitch height for test compounds are expressed as a percentage of the inhibition obtained with the standard agonist and the corresponding IC50 values determined.

The following procedure can also be used to determine the activity of agonists of δ-opioid receptors as described herein. The assay is based on the inhibition of adenylate cyclase by delta opioid receptor activation, and measures a reduction in forskolin-mediated cyclic AMP levels as a metric for receptor agonism.

Cell Culture: Chinese hamster ovary cells expressing the human δ-opioid receptor are passaged twice weekly in Ham's F-12 medium with L-glutamine containing 10% fetal bovine serum and 450 μg/mL hygromycin. Cells are prepared for assays 3 days prior to the experiment. Briefly, cells are trypsinized for passage. Viability of the cells is assessed using trypan blue, the cells counted and plated out into 96 well poly-D-lysine coated plates at a density of 7,500 cells/well.

Agonist Test Plate: Cells plated 3 days prior to assay are rinsed twice with PBS. The plates are placed into a 37° C. water bath. Fifty microliters of assay buffer (PBS, dextrose 1 mg/mL, 5 mM MgCl2, 30 mM HEPES, 66.7 μg/mL of IBMX) is then added to designated wells. Fifty microliters of appropriate drug+10 μM forskolin (final assay concentration is 5 μM forskolin) is then added to all wells, and timed for 15 minutes. The reaction is then stopped by the addition of 10 μL of 6N perchloric acid to all wells. To neutralize, 134 of 5N KOH is added to all wells, and to stabilize 12 μL of 2M Tris, pH 7.4 is added to all wells. Mix by shaking on an orbital shaker for 10 minutes, and centrifuge at setting 7 for 10 minutes. Aliquot into 3H plate.

Both test plates are placed into an Amersham 3H cAMP binding kit overnight, and harvested onto GF/B filters previously soaked in 0.5% PEI with a Skatron using 50 mM Tris HCl pH 7.4 at 4° C. Filtermats can be air-dried overnight then place in bags with 20 ml Betaplate scintillation cocktail and counted on a Betaplate counter for 60 sec per sample. Other cAMP detection/quantitation approaches can also be used. Data can be analyzed using Excel.

Acetylcholinesterase activity, and therefore acetylcholinesterase inhibitor activity, can be measured by any of a number of well-known assays. As but one example, Leuzinger et al. describe an assay in Proc. Natl Acad. U.S.A. 57: 446-451 (1967), the content of which is incorporated herein by reference. Kits for measuring acetylcholinesterase activity are commercially available, e.g., from LSBio (Cat. No. LS-K34) and from Abcam (Cat. No. Ab 138871), among others.

### EAAT1 and NCX1 Ion Channels

The Excitatory Amino Acid Transporter 1 (EAAT1), also known as Glutamate Aspartate Transporter 1 (GLAST-1) is an ion channel found in the plasma membrane of a cell and on the inner mitochondrial membrane. EAAT1 has been found to function in vivo as a homotrimer within the malate-aspartate shuttle. This channel mediates the transport of glutamic and aspartic acid with the co-transport of three Na+ and one H+ cations, and counter transport of one K+ cation. This co-transport coupling (or symport) allows the transport of glutamate into cells against a concentration gradient.

Inhibitors of EAAT1 are known in the art, and include, but are not limited to L-trans-Pyrrolidine-2,4-dicarboxylic acid, L-(−)-threo-3-Hydroxyaspartic acid, and UCPH 101.

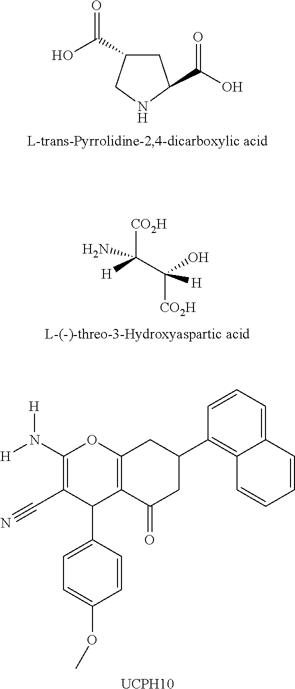

The Na⁺/Ca⁺ exchanger, referred to as NCX1 channel is an antiporter membrane protein that removes calcium from cells. The channel is additionally found at the plasma membrane of a cell and on the inner mitochondrial membrane, and is positioned in very close proximity to the EAAT1 channel NCX1 uses energy stored in the electrochemical gradient of sodium (Na+) by allowing Na+ to flow down its gradient across the plasma membrane in exchange for the counter transport of calcium ions (Ca2+). A single calcium ion is exported for the import of three sodium ions. The NCX1 ion channel is thought to be one of the most important cellular mechanisms for removing Ca2⁺.

Inhibitors of NCX1 are known in the art, and include, but are not limited KB-R7943 Mesylate, Aprindine, and SN-6. While the mesylate salt of KB-R7943 is noted here, it is specifically contemplated that other salts of KB-R7943 would have similar activity.

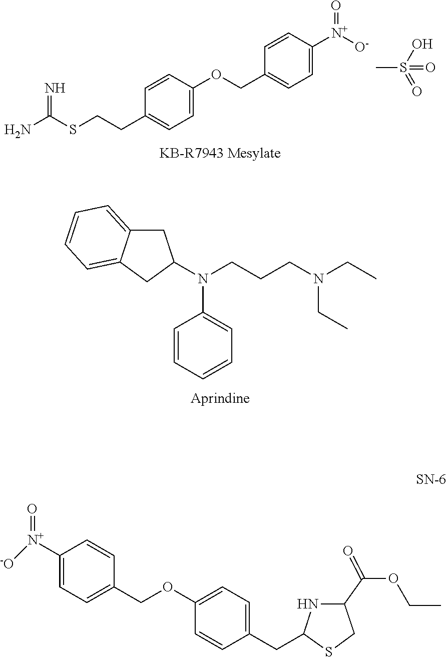

Recent evidence suggests that the EAAT1 and NCX1 channels work in concert to maintain homeostasis in a cell. Accordingly, an inhibitor for one channel can indirectly inhibit the other. In one embodiment, an inhibitor of EAAT1 channel inhibits the function of the EAAT1 channel, the NCX1 channel, or the EAAT1 and NCX1 channels. In one embodiment, an inhibitor of NCX1 channel inhibits the function of the NCX1 channel, the EAAT1 channel, or the EAAT1 and NCX1 channels.

In one embodiment, an inhibitor of the EAAT1 and/or NCX1 channels binds directly to the intended channel and alters its function.

In one embodiment, an inhibitor of the EAAT1 channel binds directly to the EAAT1 channel and alters its function, and indirectly alters the function of the NCX1 channel.

In one embodiment, an inhibitor of the NCX1 channel binds directly to the NCX1 channel and alters its function, and indirectly alters the function of the EAAT1 channel.

In one embodiment, an inhibitor of the EAAT1 and/or NCX1 channels alters channel function, for example via an allosteric regulation. As used herein, “allosteric regulation” refers to the regulation of a protein by binding at a site other than the protein's active site. In one embodiment, an inhibitor of the EAAT1 and/or NCX1 channels exhibits an allosteric regulation of the channel.

In one embodiment, an inhibitor of the EAAT1 and/or NCX1 channels changes the structure or formation of the channel and alters its function.

In one embodiment, an inhibitor of the EAAT1 and/or NCX1 channels changes the interaction between the EAAT1 and NCX1 channels and alters the function of at least one of the channels.

In certain embodiments, altering the function of the EAAT1 and/or NCX1 channels includes inhibiting the function of the EAAT1 and/or NCX1 channels, or inhibiting at least 10% of the function, e.g., transporting an ion or maintaining an ion homeostasis. In one embodiment, altering the function of the EAAT1 and/or NCX1 channels is inhibiting at least 5%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% or more of the function of the channels as compared to activity in the absence of the inhibitor. For example, an agent or agonist that alters the function of the EAAT1 and/or NCX1 channels can reduce the function by limiting the ions that are transported or can reduce the number of ions that are transported.

In certain embodiments, altering the function of the EAAT1 and/or NCX1 channels includes slowing the function of the EAAT1 and/or NCX1 channel, e.g., rate of transport. In one embodiment, altering the function of the EAAT1 and/or NCX1 channels is slowing transport by at least at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% or more as compared to activity in the absence of the inhibitor.

In certain embodiments, altering the function of the EAAT1 and/or NCX1 channels includes activating or accelerating the function of the EAAT1 and/or NCX1 channel, e.g., rate of transport. In one embodiment, altering the function of the EAAT1 and/or NCX1 channels is activating/accelerating transport by at least at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% or more, or 1×, 2×, 3×, 4×, 5×, 25×, 50×, 100×, 500×, or 1000× or more as compared to activity in the absence of the inhibitor. In order to determine the functionality of a calcium ion channel following contact with an agonist of the channel, e.g., a EAAT1 or NCX1 ion channel, one skilled in the art can use any of the standard assays used in the field. For example, a Fluo-4 Direct Calcium Assay (i.e., available via TheroFisher Therapeutics; Waltham, Mass.) measures the flux of calcium through an ion channel.

In order to assess the binding site of an agonist of an ion channel, one skilled in the art can, e.g., use thermal proteome profiling. This assay can be used to identify which proteins an agonist, e.g., SNC-80 or donepezil, physically interact with, for example, in the ion channel.

In order to assess the pathways affected by an agonist of an ion channel, one skilled in the art can, e.g., use metabolomics and transcriptomics together with proteomics. This approach can be used to identify which proteins or pathways are modulated (i.e., up- or down-regulated) in the presence of the agonist.

### Inducing Biostasis and Preservation

Provided herein is a method of inducing biostasis in a cell, tissue or organ, the method comprising contacting the cell, tissue or organ in need of preservation with an agent that alters the function of at least one ion channel selected from the group consisting of EAAT1 ion channel and NCX1 ion channel, wherein the contacted cell, tissue or organ exhibits biostasis.

Provided herein is a method of preserving viability or function of a cell, tissue or organ, the method comprising contacting the cell, tissue or organ in need of such preservation with an agent that alters the function of at least one ion channel selected from the group consisting of EAAT1 ion channel and NCX1 ion channel.

Provided herein is a method of cell, tissue or organ transplant, the method comprising contacting a donor cell, tissue or organ in situ or ex vivo with an agent that alters the function of at least one ion channel selected from the group consisting of EAAT1 ion channel and NCX1 ion channel.

Provided herein is a method of preparing a cell, tissue or organ for transplant, the method comprising contacting a donor cell, tissue or organ with an agent that alters the function of at least one ion channel selected from the group consisting of EAAT1 ion channel and NCX1 ion channel.

In one embodiment, the agent further activates the δ-opioid receptor following contact.

In one embodiment, the agent does not activate the δ-opioid receptor following contact.

Provided herein is a method of inducing biostasis in a cell, tissue or organ, the method comprising contacting the cell, tissue or organ in need of preservation with an agonist for the δ-opioid receptor, wherein the contacted cell, tissue or organ exhibits biostasis wherein the agonist is administered at a dose sufficient to alter the function of at least one ion channel selected from the group consisting of EAAT1 ion channel and NCX1 ion channel.

Provided herein is a method of inducing biostasis in a cell, tissue or organ, the method comprising contacting the cell, tissue or organ in need of preservation with SNC-80, wherein the contacted cell, tissue or organ exhibits biostasis wherein the agonist is administered at a dose sufficient to alter the function of at least one ion channel selected from the group consisting of EAAT1 ion channel and NCX1 ion channel.

Provided herein is a method of inducing biostasis in a cell, tissue or organ, the method comprising contacting the cell, tissue or organ in need of preservation with Donepezil, wherein the contacted cell, tissue or organ exhibits biostasis wherein the agonist is administered at a dose sufficient to alter the function of at least one ion channel selected from the group consisting of EAAT1 ion channel and NCX1 ion channel.

Further provided is a method of preserving viability or function of a cell, tissue or organ, the method comprising contacting the cell, tissue or organ in need of such preservation with an agonist of the δ-opioid receptor wherein the agonist is administered at a dose sufficient to alter the function of at least one ion channel selected from the group consisting of EAAT1 ion channel and NCX1 ion channel.

Provided herein is a method of inducing biostasis in a cell, tissue or organ, the method comprising contacting the cell, tissue or organ in need of preservation with any of the biostasis-inducing compositions described herein, e.g., at a concentration sufficient to induce biostasis.

Provided herein is a method of preserving viability or function of a cell, tissue or organ, the method comprising contacting the cell, tissue or organ in need of such preservation with any of the biostasis-inducing compositions described herein, e.g., at a concentration sufficient to induce biostasis and/or to reduce metabolic activity in the cell, tissue or organ, and thereby preserve the viability or functional capacity of the cell, tissue or organ.

Provided herein is a method of cell, tissue or organ transplant, the method comprising contacting a donor cell, tissue or organ in situ or ex vivo with any of the biostasis-inducing compositions described herein, e.g., at a concentration sufficient to induce biostasis and/or to reduce metabolic activity in the cell, tissue or organ.

Provided herein is a method of preparing a cell, tissue or organ for transplant, the method comprising contacting a donor cell, tissue or organ with any of the biostasis-inducing compositions described herein, e.g., at a concentration sufficient to induce biostasis and/or to reduce metabolic activity in the cell, tissue or organ.

Further provided is a method of preserving viability or function of a cell, tissue or organ, the method comprising contacting the cell, tissue or organ in need of such preservation with SNC-80, e.g., at a concentration sufficient to induce biostasis and/or to reduce metabolic activity in the cell, tissue or organ.

Further provided is a method of preserving viability or function of a cell, tissue or organ, the method comprising contacting the cell, tissue or organ in need of such preservation with Donepezil, e.g., at a concentration sufficient to induce biostasis and/or to reduce metabolic activity in the cell, tissue or organ

In one embodiment, the methods comprise further administering at least a second agent. For example, the at least a second agent can be an inhibitor of the NCX1 ion channel, or a EAAT1 inhibitor. In one embodiment, second agent is KB-R7943 mesylate.

In one embodiment, the methods comprise further administering KB-R7943 mesylate.

In certain aspects, the method comprises administering SNC-80 and KB-R7943 mesylate.

In one embodiment, induced biostasis or preservation reduces cell death and/or degradation occurring in the cell, tissue or organ, e.g., after transplantation. In one embodiment, induced biostasis or preservation reduces cell death and/or degradation in the cell, tissue or organ by at least 5, 10, 15, 20, 25, 30, 35, 40, 45, 50, 55, 60, 65, 70, 75, 80, 85, 90, 95, 99% or more as compared to an otherwise identical cell, tissue or organ that is not contacted with an agonist or agent as described herein. Cell death and/or degradation in a cell, tissue or organ can be assessed by one skilled in the art using standard techniques, for example, by visualizing trypan blue dye, a vital stain that selectively labels dead cells or tissue. Trypan blue dye is readily pumped out of healthy cells; the presence of cellular trypan blue dye indicates that the cell has undergone cellular death. Other viability assays are discussed herein below.

In one embodiment, induced biostasis or preservation delays cell death and/or degradation in the cell, tissue or organ. In one embodiment, induced biostasis or preservation delays cell death and/or degradation in the cell, tissue or organ by at least 1, 2, 3, 4, 5, 6, 7, 8, 9, 10, 11, 12, 13, 14, 15, 16, 17, 18, 19, 20, 21, 22, 23, 24 hours, or more as compared to an otherwise identical cell, tissue or organ that is not contacted by a delta opioid receptor agonist or other biostasis-inducing agent as described herein.

The viability of a cell, tissue or organ is limited following removal from a donor for transplant. For example, using current preservation approaches, a heart/lung is viable for 4 to 6 hours; a pancreas for 12 to 24 hours; a liver for up to 24 hours; a kidney for 48 to 72 hours; a cornea for 5 to 7 days; and heart valves, skin, bone, saphenous veins for 3 to 10 years. In one embodiment, preservation extends the period for which a cell, tissue or organ is viable outside the donor. In one embodiment, preservation extends the period for which a cell, tissue or organ is viable by at least 10% or more relative to an otherwise identical cell, tissue or organ treated in substantially the same way but lacking contacting with the agonist or biostasis-inducing agent. In further embodiments, preservation extends the period for which a cell, tissue or organ is viable by at least 20%, 40%, 60%, 80%, 100% (i.e. doubling the period), 120%, 140%, 160%, 180%, 200% (i.e., tripling the period), or more relative to an otherwise identical cell, tissue or organ treated in substantially the same manner but lacking contacting with the agonist or biostasis-inducing agent. One skilled in the art can determine if a cell, tissue or organ is viable using a viability assay as described herein below.

In one embodiment, the tissue is selected from the group consisting of cornea, bone, cartilage, tendon, pancreas islet, heart valve, nerve, vascular, deep tissue flap, fat tissue, muscle, and vein.

In one embodiment, the organ is selected from the group consisting of intestine, stomach, heart, kidney, bladder, pancreas, liver, lung, brain, skin, uterus, digit, and limb.

In one embodiment, the cell, or population thereof, is a stem cell, a T cell, an embryonic stem cells, an induced pluripotent cell, a differentiated cell, an organoid, or a primary cell. In one embodiment, the cell is an engineered cell, e.g., a Chimeric Antigen Receptor (CAR) cell, e.g., a CAR T cell.

A viability assay permits the qualitative or quantitative determination of the viability of a tissue, organ, or individual cells. However, the type of viability assay needed can depend upon the cellular or tissue structure of the material being evaluated. One skilled in the art can determine whether cells are viable within a sample using any of the assays described herein below.

Dye exclusion viability assays begin by creating a cellular suspension in which a dye is introduced; e.g., naphthalene black, erythrosine, or trypan blue. Once the dye has been introduced, the solution will be examined using, e.g., an optical microscope optionally with a hemocytometer. Viable, whole cells exclude and will not be able to be penetrated by these dyes, however, the cells that have been damaged beyond recovery will permit dye entry visible under a microscope. By counting viable and non-viable cells, e.g., in a microscopic field or on the grid of a hemocytometer, one can determine the properties or percentage of viable versus non-viable cells. Once the dye has permeated, it will be able to determine which percentage of the cells within the test sample was viable and which percentage were not. Dye exclusion viability assays are useful when testing a sample to determine the amount of cells still viable.

Dye Uptake Viability Assays introduce a dye that can only be taken up by healthy cells, rather than dyes that are rejected by healthy cells. A neutral red staining can be introduced to a sample that will penetrate a living cell, but that will not penetrate dead or dying cells. Once the red stain has been introduced, the sample can be viewed, e.g., through the use of an optical microscope. The viability of the sample can then be expressed as a percentage of viable cells.

Fluorescent viability assays use for example, diacetyl fluorescein, which is hydrolyzed into fluorescein. This assay gives cells a fluorescence under the correct light. Only healthy cells will be able to give off a green fluorescence. Propidium iodine or ethidium bromide, which fluorescent when bound to DNA but excluded by viable cells, can be used to fluorescently quantitate cell viabilityin a manner similar to trypan blue dye exclusion when viewed under a fluorescence microscope. Fluorescence-based assays, whether using dye uptake or dye exclusion, can also be adapted to measure the intensity of fluorescence in a sample, rather than fluorescence of individual cells.

Mitochondrial assays are generally used when cell death may be imminent or on-going. A mitochondrial assay can, for example, separate different stages of the apoptosis process, utilizing, for example, Resazurin and Formazan. This assay is often used when it is suspected that cell death in a tissue or an organ could be on-going.

Functional assays can be performed dependent on the specific cell type, tissue or organ being tested. As but one example, red blood cells can be tested for viability during medical procedures. A functional assay will be based on the normal function of such specific cells. For example, red blood cells may be assayed not only regarding whether they are viable or non-viable, but also whether they are deformed, whether they are fragile, and whether they have the right ATP level and hemoglobin content. Other tissues or organs generate markers of the death of specific cells. For example, measurement of cardic enzymes (e.g., troponin T (TnT) Troponin I (TnI, and creatine phosphokinase) in the blood provides a measure of these normally intracellular cardiac-specific enzymes that have been liberated from cardiac cells, and thereby a measure of cardiac/cardiomyocyte cell death. The measurement of enzymes of this type in the extracellular environment can provide a measure of the viability of cardiac tissue of a donor heart. Similar markers for, e.g., renal damage or viability include, for example, serum cystatin C and serum neutrophil gelatinase—associated lipocalin. Any of these markers in, e.g., a perfused kidney, or in extracellular locations in the kidney can provide a measure of renal viability or donor tissue quality. Finally, assays may be performed on organs in entirety simply through the process of transplantation.

In one embodiment, biostasis results in the modulation of the gene profile of the cell, tissue or organ, such that biostasis is achieved. For example, when biostasis is induced in a cell, tissue or organ as a result of contacting the cell, tissue or organ with an agonist of delta-opioid receptor, at least one gene listed in Table 1 is modulated, e.g., upregulated or downregulated. Modulation is with respect to a reference level. Such modulation of at least one such gene can be seen when biostasis is induced, for example with SNC-80. Modulation of at least one such gene can also be seen when biostasis is induced with Donepezil. In one embodiment, the at least one gene selected from Table 1 is upregulated by at least 1%, 5%, 10%, 15%, 20%, 25%, 30%, 35%, 40%, 45%, 50%, 55%, 60%, 65%, 70%, 75%, 80%, 85%, 90%, 95%, 99%, or more, or at least 1-fold, 5-fold, 10-fold, 15-fold, 20-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, 100-fold, or more as compared to a reference level. In one embodiment, the at least one gene selected from Table 1 is downregulated by at least 1%, 5%, 10%, 15%, 20%, 25%, 30%, 35%, 40%, 45%, 50%, 55%, 60%, 65%, 70%, 75%, 80%, 85%, 90%, 95%, 99%, or more as compared to a reference level. As used herein, a “reference level” refers to the level of gene or gene product expression in an otherwise identical sample that is not contacted with an agonist or other agent as described herein. A skilled person can measure the gene or gene product expression using standard techniques, e.g., PCR based assays or western blotting to assess mRNA and protein levels, respectively. In one embodiment, at least 5% of the genes listed in Table 1 are modulated when biostasis is induced. In another embodiment, at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more of the genes listed in Table 1 are modulated when biostasis is induced.

In one embodiment, biostasis results in the modulation of oxygen consumption metrics (e.g., VO2), pulse oximetry, blood assays (e.g., ATP/ADP ratio, adenylate kinase, alkaline phosphatase, lactate dehydrogenase, heme oxygenase, alanine aminotransferase, aspartate aminotransferase), temperature, respirometry. One skilled in the art can determine if these factors have been modulated using standard techniques in the art. In one embodiment, one can assess clinical metrics of cell, tissue or organ damage, for example, blood lactate dehydrogenase, superoxide dismutase, pH, creatinine, cognitive function, to determine if biostasis was induced.

In one embodiment, a δ-opioid receptor agonist, SNC-80 or a derivative or analogue thereof, or Donepezil or a derivative or analogue thereof or an EAAT1 or NCX1 ion channel modulator suppresses metabolism via internal molecular mechanisms in a stable, reversible manner for stabilization of cells, tissues, organs, tissues, and whole organisms.

In one embodiment, the cell, tissue or organ can be contacted with an agonist or other agent as described herein for a duration sufficient to induce biostasis or preservation. In one embodiment, the cell, tissue or organ is contacted for at least 1 minute to initiate biostasis or preservation. In other embodiments, contacting is for at least 5 minutes, at least 10, 20, 30, 40, 50, or 60 minutes (1 hour) or more, e.g., at least 2, 3, 4, 5, 6, 7, 8, 9, 10, 11, 12, 13, 14, 15, 16, 17, 18, 19, 20, 21, 22, 23, 24 hours or more; at least 1, 2, 3, 4, 5, 6, 7 days or more; at least 1, 2, 3, 4, 5, 6 weeks or more. In one embodiment, the contacting occurs for a fraction of normal metabolic rate of a given cell, tissue or organ. Once biostasis is induced, the cells, tissue, organ or organism can be maintained in that state by continuing contact with the agonist or other agent. Contacting can be maintained for as long as biostasis or preservation is needed. Length of effective biostasis or preservation will vary with, for example, the type of cell, tissue, organ or organism, as well as other factors, such as the original state of the cell, tissue, organ or organism, and other treatments used in combination with the δ-opioid receptor agonist or other agent that induces biostasis. In one embodiment, markers of, e.g., necrotic or apoptotic cell death can be measured to monitor the status of the preserved material. During or after, e.g., transplantation, the agonist can be withdrawn or no longer administered so as to permit reversal of biostasis.

In one embodiment, the contacting with a δ-opioid receptor agonist, SNC-80, Donepezil or other EAAT1 and/or NCX1 modulator(s) is short-term, e.g., less than 24 hours. In an alternative embodiment, the contacting is long-term, e.g., greater than or equal to 24 hours. One skilled in the art can determine if contact has induced biostasis or preservation by determining if, for example, the gene profile or gene product expression described herein above is achieved, or via other measures or assays of viability or function known to those skilled in the art or described herein.

In one embodiment, the δ-opioid receptor agonist, SNC-80, Donepezil or modulator(s) of EAAT1 and/or NCX1 as described herein can be used as part of a storage solution for living cells, cultured tissues, tissue explants, or organ explants using immersion methods for tissues/organs that cannot be perfused.

In one embodiment, contacting is performed via perfusion. As used herein, “perfusion” refers to the act of pumping or passing a fluid through an organ or tissue, preferably the passage of fluid through the vasculature of an organ or tissue.

In one embodiment, contacting is performed via immersion, that is, by placing the cell, tissue or organ in a solution comprising the agonist or other agent(s) such that the cell, tissue or organ is completely covered by the solution. In one embodiment, contacting is performed via direct introduction, e.g., placement, to the cell, tissue or organ. As a non-limiting example, perfusion of a donor kidney by introducing the agonist directly into the renal circulation before removal of the kidney would be considered direct introduction. A combination of perfusion and immersion, whether simultaneous or concurrent (e.g., perfuse first, then store immersed, or perfuse while immersed) can also be used.

In one embodiment, perfusion or immersion is performed in vivo, e.g., prior to removal of a donor organ or tissue, or ex vivo.

In one embodiment, the method further comprises contacting the cell, tissue or organ with at least a second biostatic composition or compound. In one embodiment, the second composition or compound is selected from hydrogen sulfide; nitrogen, argon, or other gases with or without addition of oxygen to modulate oxygen in tissue; Oligomycin A, rotenone, or other electron transport chain inhibitors known in the art; 2-deoxyglucose and other glycolysis inhibitors known in the art; adenosine monophosphate (AMP); a neuropeptide; deferoxamine; an antioxidants or anti-inflammatory agents known in the art; and a prolyl hydroxylase inhibitor.

Hydrogen sulfide is the chemical compound with the formula H₂S. Hydrogen sulfide, a gasotransmitter, has been shown recently to have therapeutic properties for treatment in, e.g., diabetes, cardiovascular disease, and neurodegenerative disease. Therapeutic uses of hydrogen sulfide are reviewed in, e.g., Y D, Wen, et al. Oxidative Medicine and Cellular Longevity; Volume 2018, Article ID 4010395; and Jansen A R, SHOCK; 2017: 48(5); and Li, Z, et al. Circulation Research. 2018; 123:590-600, the contents of which are incorporated herein by reference in their entireties.

AMP, or 5′-adenylic acid, is a nucleotide which consists of a phosphate group, a sugar ribose, and a nucleobase adenine; it is an ester of phosphoric acid and the nucleoside adenosine. AMP plays an important role in many cellular metabolic processes, being interconverted to ADP and/or ATP, and is also a component in synthesis of RNA.

AMP is also known by its chemical name, [(2R,3S,4R,5R)-5-(6-aminopurin-9-yl)-3,4-dihydroxyoxolan-2-yl]methyl dihydrogen phosphate, and has the chemical structure:

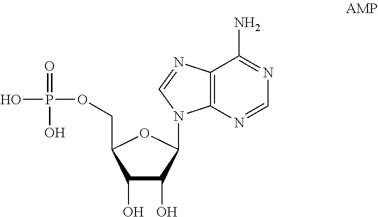

Deferoxamine, otherwise known as desferrioxamine or desferal, is a chelating agent that can be used to remove excess iron or aluminum from the body, organ or tissue. In that capacity, it acts by binding free iron and reducing ischemia-induced free radical damage. By removing excess iron or aluminum, the agent reduces the damage done to various organs and tissues, such as the liver. For example, 100 mg of deferoxamine is capable of binding approximately 8.5 mg of trivalent (ferric) iron. The iron chelation effect also induces hypoxia response pathways by preventing prolyl 4-hydroxylase degradation of HIF 1, thus mimicking a low-oxygen state.

Deferoxamine is also known by its chemical name, N-(5-aminopentyl)-N-hydroxy-N′-[5-(N-hydroxy-3-{[5-(N-hydroxyacetamido)pentyl]carbamoyl}propanamido)pentyl]butanediamide, and has the chemical structure:

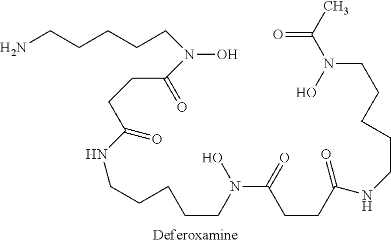 [SMILES: CC(=O)N(O)CCCCCNC(=O)CCC(=O)N(O)CCCCCNC(=O)CCC(=O)N(O)CCCCCN]

In one embodiment, a cell, tissue or organ is contacted with a δ-opioid receptor agonist, SNC-80, Donepezil or EAAT1 and/or NCX1 modulator and the second compound at substantially the same time. In an alternative embodiment, the cell, tissue or organ are contacted with the agonist or other agent and the second compound at different times.

While cooling is not necessary in some embodiments, in one embodiment, the cell, tissue or organ is contacted with the agonist or other agent under hypoxia, osmotic stress, physiological stress, burn injury, blast injury, trauma, radiation, chemical exposure, toxin exposure and cooling or freezing condition. A hypoxic condition refers to a state in which oxygen supply is insufficient in the cell, tissue or organ. A cooling condition refers to state in which the internal temperature is decreased from the normal temperature, e.g., 37° C. or 98.6° F.

In one embodiment, induced biostasis or preservation is reversed upon withdrawal of the agonist or other agent, or upon administration of an agent that counters the effect thereof, e.g., a delta-opioid receptor antagonist, as the case may be. Opioid antagonist, e.g., a delta-receptor opioid antagonists, are known in the art and can be identified by a skilled person. Exemplary delta-opioid receptor antagonists are further reviewed in, U.S. Pat. Nos. 4,816,586; 5,631,263; 5,411,965; 5,352,680; and 8,980,908, the contents of which are incorporated herein by reference in their entireties.

In one embodiment, contacting does not induce hypothermia. As used herein, “hypothermia” refers the state in which the internal temperature of, e.g., a tissue, organ or subject, is lower than 95° F. One skilled in the art can determine if hypothermia has occurred by assessing, e.g., the internal temperature of the tissue, organ or subject.

In one embodiment, the δ-opioid receptor agonist, SNC-80, Donepezil, or EAAT1 and/or NCX1 modulator(s) is not contacted with cells, tissue or an organ in combination with a local anesthetic. In another embodiment, the agonist or other agent is not contacted in combination with an anti-arrhythmic agent. In another embodiment, the agonist or other agent is not contacted in combination with citrate. In another embodiment, the agonist or other agent is not contacted in combination with exogenous magnesium. In another embodiment, the agonist or other agent is not contacted in combination with a local anesthetic, anti-arrhythmic agent, exogenous citrate or exogenous magnesium.

The methods and compositions for inducing stasis as described herein can also be used to preserve cells, tissues or organs for transport or shipment. In one embodiment, cells, tissues or organs can be preserved by inducing stasis as described herein prior to shipment, e.g., via, courier, mail or parcel carrier. This approach can not only increase viable lifespan of a cell, tissue or organ for transplant or other use, but can also obviate or lessen the need to cold-chain shipment, whether refrigerated, on ice or on dry ice or under liquid nitrogen.

The methods and compositions for inducing stasis as described herein can also be used to preserve cells, tissues or organs for storage, for example, liquid nitrogen storage.

The methods and compositions for inducing stasis as described herein can also be used to preserve cells during cell passage. As used herein, “cell passage” refers to a cell culture technique used to maintain live cells under culture conditions for extended periods of time.

The methods and compositions for inducing stasis as described herein can also be used to preserve a whole organism, e.g., a non-mammal organism, a non-human mammal, or a human.

### Reversing Biostasis and Preservation

In order for a preserved cell, tissue or organ as described herein to function as needed, biostasis as described herein must be reversible. In one aspect, reversing the biostasis induced with an agent or agents as described herein is a matter of removal of the agent(s). This can be achieved by simply transplanting the preserved tissue to a recipient, where the recipient and the transplanted tissue are not administered the agent(s). In this passive approach, dilution of the agent(s) by the recipient's own circulation will remove the agent(s) and thereby reverse biostasis.

In other approaches, the reversal can comprise, for example, active treatment of the preserved cells, tissue or organ to either remove the biostasis-inducing agent(s) prior to transplant, and/or to counteract the biostasis-indjcing agent(s) prior to transplant. Removal can be performed, for example, by perfusion with and/or immersion in a medium lacking the biostasis inducing agent(s). Counteracting the agent(s) can be achieved by contacting the cells, tissue or organ with one or more different agents that inhibit or counter the biotatitic effect. As with induction of biostasis, the contacting with a counteracting agent or agents can be performed by perfusion, immersion or a combination of both.

Accordingly, provided herein is a method of restoring metabolic activity in a cell, tissue or organ that has been contacted by an agonist of the δ-opioid receptor, wherein the agonist is administered at a dose sufficient to alter the function of at least one ion channel selected from the group consisting of EAAT1 ion channel and NCX1 ion channel or an agent that alters the function of at least one ion channel selected from the group consisting of EAAT1 ion channel and NCX1 ion channel, the method comprising contacting the cell, tissue or organ with a polyol.

Provided herein is a method of restoring oxidative metabolism in a cell, tissue or organ that has been contacted by an agonist of the δ-opioid receptor, wherein the agonist is administered at a dose sufficient to alter the function of at least one ion channel selected from the group consisting of EAAT1 ion channel and NCX1 ion channel or an agent that alters the function of at least one ion channel selected from the group consisting of EAAT1 ion channel and NCX1 ion channel, the method comprising contacting the cell, tissue or organ with a polyol.

Provided herein is a method of restoring normal metabolic function in a cell, tissue or organ that has been contacted by an agonist of the δ-opioid receptor, wherein the agonist is administered at a dose sufficient to alter the function of at least one ion channel selected from the group consisting of EAAT1 ion channel and NCX1 ion channel or an agent that alters the function of at least one ion channel selected from the group consisting of EAAT1 ion channel and NCX1 ion channel, the method comprising contacting the cell, tissue or organ with a polyol.

Provided herein is a method for reversing biostasis comprising removing an agonist or agent that induced biostasis from contact with the organ or tissue.

In one embodiment, reversal of biostasis results in restoring the gene profile of the organ or tissue to the gene profile prior to contact by an agent or agonist. As described herein above, biostasis can be defined by a modulation of at least one gene selected from Table 1; reversal of the biostasis would revert the modulation of the at least one gene. The gene profile of an organ or tissue can be assessed using methods described herein above.

In one embodiment, reversal of biostasis results in restoring the oxygen consumption levels of the organ or tissue to what it was prior to contact by an agent or agonist. Biostasis can be defined a reduction in oxygen consumption; reversal of the biostasis would result in an increased oxygen consumption as compared to biostasis levels, with levels preferably at or comparable to the levels in a normally metabolically active organ or tissue. The oxygen consumption of an organ or tissue can be assessed using methods described herein above. In one embodiment, oxygen consumption is increased by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or more, or at least 1×, at least 2×, at least 3×, at least 4×, at least 5×, at least 6×, at least 7×, at least 8×, at least 9×, at least 10×, at least 11×, at least 12×, at least 13×, at least 14×, at least 15×, at least 50×, at least 100×, at least 500×, at least 1000× or more as compared to the oxygen consumption levels at biostasis.

In one embodiment, reversal of biostasis results in restoring the metabolic function of the organ or tissue to what it was prior to contact with an agent or agonist. Biostasis generally involves a reduction in metabolic function; reversal of the biostasis would result in an increased metabolic function as compared to biostasis levels. The metabolic function of an organ or tissue can be assessed using methods described herein above. In one embodiment, metabolic function is increased by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or more, or at least 1×, at least 2×, at least 3×, at least 4×, at least 5×, at least 6×, at least 7×, at least 8×, at least 9×, at least 10×, at least 11×, at least 12×, at least 13×, at least 14×, at least 15×, at least 50×, at least 100×, at least 500×, at least 1000× or more as compared to the oxygen consumption levels at biostasis.

In one aspect, biostasis or preservation is reversed by removing the agent or agonist that was used to contact the organ or tissue in order to induce biostasis or preservation.

In one embodiment, the method comprises adding a polyol directly to the composition in contact with the organ or tissue, i.e., not removing the agonist or agent in contact with the organ or tissue prior to contact.

In one embodiment, the method comprises the step, prior to contact with the polyol, of removing the agonist or agent from the organ or tissue.

In one embodiment, the polyol is kestose or erlose.

### Cell, Tissue or Organ Transplant

One aspect described herein provides a method of cell, tissue or organ transplant, the method comprising contacting a donor cell, tissue or organ in situ or ex vivo with one or more agents as described herein that induce biostasis.

Another aspect described herein provides a method of cell, tissue or organ transplant, the method comprising contacting a donor cell, tissue or organ in situ or ex vivo with SNC-80.

Another aspect described herein provides a method of cell, tissue or organ transplant, the method comprising contacting a donor cell, tissue or organ in situ or ex vivo with Donepezil.

Another aspect herein is a method of preparing a cell, tissue or organ for transplant, the method comprising contacting a donor cell, tissue or organ with an agonist of the δ-opioid receptor.

Another aspect described herein is a method of preparing a cell, tissue or organ for transplant, the method comprising contacting a donor cell, tissue or organ with SNC-80.

Another aspect described herein is a method of preparing a cell, tissue or organ for transplant, the method comprising contacting a donor cell, tissue or organ with Donepezil.

Dosages or concentrations for biostasis-inducing agents are discussed elsewhere herein.

The term “donor,” refers to mammalian species from which a transplant is obtained. In one embodiment, the donor is deceased, e.g., has been pronounced clinically deceased prior to contacting. In one embodiment, the donor is brain dead. In one embodiment, the donor is a live donor. A living donor remains alive and donates a renewable tissue, cell, or fluid (e.g., blood, skin), or donates an organ or part of an organ in which the remaining organ can regenerate or take on the workload of the rest of the organ (primarily single kidney donation, partial donation of liver, lung lobe, small bowel).

Methods described herein can be used to induce biostasis or preservation of any cell, tissue or organ that is capable of being transplanted in a recipient, and can be used in any type of transplant.

Autografts are a transplant of tissue to the same person. For example, a transplant done with surplus tissue, tissue that can regenerate, or tissues more needed elsewhere (e.g., skin grafts, vein extraction for CABG, etc.). An autograft can be done to remove a tissue and then treat it or the person before returning it (e.g., stem cell autograft and storing blood in advance of surgery). In a rotationplasty, a distal joint is used to replace a more proximal one; typically a foot or ankle joint is used to replace a knee joint. The subject's foot is severed and reversed, the knee removed, and the tibia joined with the femur.

Allografts are a transplant of an organ or tissue between two genetically non-identical members of the same species. The majority of human tissue and organ transplants are allografts. Due to the genetic difference between the organ and the recipient, the recipient's immune system can identify the organ as foreign and attempt to destroy it, resulting in transplant rejection. The risk of transplant rejection can be estimated by measuring the Panel reactive antibody level and be treated or prevented.

Isografts are a subset of allografts in which organs or tissues are transplanted from a donor to a genetically identical recipient (such as an identical twin). Isografts are differentiated from other types of transplants because while they are anatomically identical to allografts, they do not trigger an immune response.

Xenografts are transplants of organs or tissue from one species to another. An example is porcine heart valve transplant, which is quite common and successful. Another example is attempted piscine-primate (fish to non-human primate) transplant of islet (i.e. pancreatic or insular tissue) tissue.

Domino transplants are transplant of at least two organs in one procedure. For example, in people with cystic fibrosis (CF), where both lungs need to be replaced, it is a technically easier operation with a higher rate of success to replace both the heart and lungs of the recipient with those of the donor. As the recipient's original heart is usually healthy, it can then be transplanted into a second recipient in need of a heart transplant, thus making the person with CF a living heart donor. Another example of this situation occurs with a special form of liver transplant in which the recipient suffers from familial amyloidotic polyneuropathy, a disease where the liver slowly produces a protein that damages other organs. The recipient's liver can then be transplanted into an older person for whom the effects of the disease will not necessarily contribute significantly to mortality. This term also refers to a series of living donor transplants in which one donor donates to the highest recipient on the waiting list and the transplant center utilizes that donation to facilitate multiple transplants. These other transplants are otherwise impossible due to blood type or antibody barriers to transplantation. The “Good Samaritan” kidney is transplanted into one of the other recipients, whose donor in turn donates his or her kidney to an unrelated recipient. Depending on the person on the waiting list, this has sometimes been repeated for up to six pairs, with the final donor donating to the person at the top of the list. This method allows all organ recipients to get a transplant even if their living donor is not a match to them.

Because very young children (generally under 12 months, but often as old as 24 months) do not have a well-developed immune system, it is possible for them to receive organs from otherwise incompatible donors. This is known as ABO-incompatible (ABOi) transplantation. Graft survival and recipient mortality is approximately the same between ABOi and ABO-compatible (ABOc) recipients. While focus has been on infant heart transplants, the principles generally apply to other forms of solid organ transplantation. The most important factors are that the recipient not have produced isohemagglutinins, and that they have low levels of T cell-independent antigens. United Network for Organ Sharing (LINOS) regulations allow for ABOi transplantation in children under two years of age if isohemagglutinin titers are 1:4 or below, and if there is no matching ABOc recipient. Studies have shown that the period under which a recipient may undergo ABOi transplantation may be prolonged by exposure to nonself A and B antigens.

Exemplary cell, tissue or organs that can be successfully transplanted from a deceased or living donor include Heart (deceased-donor, except as noted above), Lung (deceased-donor and living-related lung transplantation), Heart/Lung (deceased-donor and domino transplant), Kidney (deceased-donor and living-donor), Liver (deceased-donor enables donation of a whole liver; and living-donor provides partial liver), Pancreas (deceased-donor only), Intestine (deceased-donor and living-donor; normally refers to the small intestine), Stomach (deceased-donor only) Testis (deceased-donor and living-donor), Penis (deceased-donor only), Hand (deceased-donor only), Cornea (deceased-donor only), Skin, including face replant (autograft) and face transplant, Islets of Langerhans (pancreas islet cells) (deceased-donor and living-donor), Bone marrow/Adult stem cell (living-donor and autograft), Blood transfusion/Blood Parts Transfusion (living-donor and autograft), Blood Vessels (autograft and deceased-donor), Heart Valve (deceased-donor, living-donor and xenograft (porcine/bovine)), and Bone (deceased-donor and living-donor)

In one embodiment, the tissue or organ is contacted prior to removal from the donor for a transplant in a recipient.

In one embodiment, the cell, tissue or organ is contacted following removal from the donor, and prior to a transplant in a recipient.

In one embodiment, contacting protects the cell, tissue or organ from injury prior to transplantation.

In one embodiment, the cell, tissue or organ is contacted following an injury to the cell, tissue or organ. In one embodiment, the cell, tissue or organ is contacted prior to a surgical procedure.

### Composition

One aspect provided herein is a composition comprising at least two agents that alter the function of at least one ion channel selected from the group consisting of EAAT1 ion channel and NCX1 ion channel. In one embodiment, the composition comprises at least 2, 3, 4, 5, or more agents that alter the function of at least one ion channel selected from the group consisting of EAAT1 ion channel and NCX1 ion channel.

One aspect provided herein is a composition comprising an agonist of the δ-opioid receptor and an agent that alters the function of at least one ion channel selected from the group consisting of EAAT1 ion channel and NCX1 ion channel. For example, the composition comprises SNC-80 and Donepezil.

In one embodiment, the composition is a unit dosage that results in higher than in vivo therapeutic concentration of the agent, or agents, and is sufficient to alter the function of the EAAT1 and/or NCX1 ion channels.

In one embodiment, the composition further comprises deuterium oxide. Deuterium oxide, otherwise known as “heavy water,” is a form of water that contains only deuterium (2H or D, also known as heavy hydrogen) rather than the common hydrogen-1 isotope CH or H, also called protium), which is a common component of normal water. The presence of the heavier hydrogen isotope gives the heavy water different nuclear, physical and chemical properties when compared to normal water. Methods for producing deuterium oxide are known in the art, and are further described in, e.g., U.S. Pat. No. 2,690,379, the contents of which are incorporated herein by reference in its entirety. Compositions comprising deuterium oxide are described in, e.g., U.S. Pat. No. 6,376,531, the contents of which are incorporated herein by reference in its entirety.

One aspect provided herein is a composition comprising deuterium oxide and an agent that alters the function of at least one ion channel selected from the group consisting of EAAT1 ion channel and NCX1 ion channel, e.g., Donepezil.

In one embodiment, deuterium oxide is used at a concentration of at least 10% of the composition. In one embodiment, deuterium oxide is used at a concentration of at least 5%, at least 15%, at least 20%, at least 25%, at least 30%, at least 35%, at least 40%, at least 45%, at least 50% of the composition. In one embodiment, deuterium oxide is used at a concentration range of 10-50%, 20-50%, 30-50%, 40-50%, 10-40%, 10-30%, 10-20%, 10-40%, 20-40%, 20-50%, 20-30%, or 25-45%.

One aspect provided herein is a composition comprising deuterium oxide and an agonist of the δ-opioid receptor, e.g., SNC-80.

One aspect provided herein is a composition comprising a live explanted cell, tissue or organ in contact with a δ-opioid receptor agonist, wherein the agonist is present in an amount sufficient to alter the function of at least one ion channel selected from the group consisting of EAAT1 ion channel and NCX1 ion channel; or an agent that alters the function of at least one ion channel selected from the group consisting of EAAT1 ion channel and NCX1 ion channel.

In one embodiment, the composition further comprises at least a second agent that alters the function of at least one ion channel selected from the group consisting of EAAT1 ion channel and NCX1 ion channel

In one embodiment, the composition further comprises deuterium oxide.

One aspect provided herein is a composition comprising a live explanted cell, tissue or organ in contact with SNC-80.

One aspect provided herein is a composition comprising a live explanted cell, tissue or organ in contact with Donepezil.

Another aspect provided herein is a live explanted cell, tissue or organ in biostasis induced by contact with an exogenous δ-opioid receptor agonist.

Another aspect provided herein is a live explanted cell, tissue or organ in biostasis induced by contact with SNC-80.

Another aspect provided herein is a live explanted cell, tissue or organ in biostasis induced by contact with Donepezil.

In one embodiment of either of these aspects, the cell, tissue or organ is a human cell, tissue or organ.

In one embodiment, the composition further comprises a pharmaceutically acceptable carrier. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. In some embodiments of any of the aspects, a pharmaceutically acceptable carrier can be a carrier other than water. In some embodiments of any of the aspects, a pharmaceutically acceptable carrier can be an artificial or engineered carrier, e.g., a carrier in which the active ingredient would not be found to occur in nature.

In one embodiment, the composition does not further comprise local anesthetic, an anti-arrhythmic, exogenous citrate, or exogenous magnesium.

In one embodiment, the cell, tissue or organ is of human origin. In an alternate embodiment, the cell, tissue or organ is of non-human origin.

### Methods of Slowing a Viral Infection

Provided herein is a method of slowing viral replication or a viral infection in a subject, the method comprising administering SNC-80 to a subject in need thereof.

Provided herein is a method of slowing viral replication or a viral infection in a subject, the method comprising administering Donepezil to a subject in need thereof.

Provided herein is a method of slowing viral replication or a viral infection in a subject, the method comprising administering an δ-opioid receptor agonist to a subject in need thereof, wherein the agonist is administered at a dose sufficient to alter the function of at least one ion channel selected from the group consisting of EAAT1 ion channel and NCX1 ion channel.

In one embodiment, the subject has been diagnosed as having a viral infection. In one embodiment, the methods further comprise the step of diagnosing a subject as having a viral infection prior to administration. In one embodiment, the methods further comprise the step of receiving the results of an assays that diagnoses a subject as having a viral infection prior to administration. One skilled in the art can diagnose a subject as having a viral infection using assays known in the art, for example, an assay that detects a viral nucleic acid, an antibody test or a viral antigen test, or the like.

In one embodiment, the subject is at risk of having or developing a viral infection. Risk factors for a viral infection include, but are not limited to, having direct contact or close contact with a subject having a viral infection, having direct contact or close contact with an object harboring live infection-causing viruses, having a reduced immune system, poor hygiene, and living in a densely populated area.

In one embodiment, the viral infection is slowed by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more, as compared to an untreated control (e.g., a substantially similar viral infection in a tissue, organ or individual not contacted with a drug or agent, e.g., a biostasis-inducing drug or agent as described herein).

In one embodiment, slowing a viral infection refers to slowing the viral replication. In one embodiment, slowing the viral infection refers to slowing the spread of the infection from a primary site of infection.

In one embodiment, the administration is local, for example, directly to the site of infection. In one embodiment, the administration is systemic.

Provided herein is a method of slowing viral replication or a viral infection in an organ or tissue, the method comprising contacting the organ or tissue with SNC-80.

Provided herein is a method of slowing viral replication or a viral infection in an organ or tissue, the method comprising contacting the organ or tissue with Donepezil.

Provided herein is a method of slowing viral replication or a viral infection in an organ or tissue, the method comprising contacting the organ or tissue with a δ-opioid receptor agonist, wherein the agonist is administered at a dose sufficient to alter the function of at least one ion channel selected from the group consisting of EAAT1 ion channel and NCX1 ion channel

### Methods of Treating Cancer

One aspect herein provides a method of preserving healthy cells in a subject undergoing a cancer treatment comprising administering to the subject receiving or to receive an anti-cancer therapy an agonist of the δ-opioid receptor.

One aspect herein provides a method of preserving healthy cells in a subject undergoing a cancer treatment comprising administering to the subject receiving or to receive an anti-cancer therapy SNC-80.

One aspect herein provides a method of preserving healthy cells in a subject undergoing a cancer treatment comprising administering to the subject receiving or to receive an anti-cancer therapy Donepezil.

In one embodiment, administering is performed prior to, at substantially the same time, and/or after receiving an anti-cancer therapy.

In one embodiment, the agonist of δ-opioid receptor, SNC-80, or Donepezil is administered in combination with a cancer treatment, e.g., an anti-cancer therapy, e.g., a treatment for the intended use of treating a subject with cancer. An anti-cancer therapy can be, e.g., chemotherapy, radiation therapy, chemo-radiation therapy, immunotherapy, hormone therapy, surgery or stem cellular therapy, or an engineered tissue construct. In one embodiment, the anti-cancer therapy is high dose or high exposure treatment; including, for example, treatment at a dose or exposure that would normally be lethal if not for a protective effect of the δ-opioid receptor agonist, SNC-80, or Donepezil.

In accordance with one embodiment, the subject is administered a chemotherapeutic agent in combination with an agonist of δ-opioid receptor, SNC-80, or Donepezil as described herein. Exemplary chemotherapeutic agents include, but are not limited to, a platinum chemotherapeutic agent, an anthracycline therapeutic agent, or an alkylating chemotherapeutic agent. Non-limiting examples of chemotherapeutic agents include an anthracycline (e.g., doxorubicin (e.g., liposomal doxorubicin)), a vinca alkaloid (e.g., vinblastine, vincristine, vindesine, vinorelbine), an alkylating agent (e.g., cyclophosphamide, decarbazine, melphalan, ifosfamide, temozolomide), an immune cell antibody (e.g., alemtuzamab, gemtuzumab, rituximab, tositumomab), an antimetabolite (including, e.g., folic acid antagonists, pyrimidine analogs, purine analogs and adenosine deaminase inhibitors (e.g., fludarabine)), an mTOR inhibitor, a TNFR glucocorticoid induced TNFR related protein (GITR) agonist, a proteasome inhibitor (e.g., aclacinomycin A, gliotoxin or bortezomib), an immunomodulator such as thalidomide or a thalidomide derivative (e.g., lenalidomide). General chemotherapeutic agents considered for use in combination therapies include anastrozole (Arimidex®), bicalutamide (Casodex®), bleomycin sulfate (Blenoxane®), busulfan (Myleran®), busulfan injection (Busulfex®), capecitabine (Xeloda®), N4-pentoxycarbonyl-5-deoxy fluorocytidine, carboplatin (Paraplatin®), carmustine (BiCNU®), chlorambucil (Leukeran®), cisplatin (Platinol®), cladribine (Leustatin®), cyclophosphamide (Cytoxan® or Neosar®), cytarabine, cytosine arabinoside (Cytosar-U®), cytarabine liposome injection (DepoCyt®), dacarbazine (DTIC-Dome®), dactinomycin (Actinomycin D, Cosmegan), daunorubicin hydrochloride (Cerubidine®), daunorubicin citrate liposome injection (DaunoXome®), dexamethasone, docetaxel (Taxotere®), doxorubicin hydrochloride (Adriamycin®, Rubex®), etoposide (Vepesid®), fludarabine phosphate (Fludara®), 5-fluorouracil (Adrucil®, Efudex®), flutamide (Eulexin®), tezacitibine, Gemcitabine (difluorodeoxycitidine), hydroxyurea (Hydrea®), Idarubicin (Idamycin®), ifosfamide (IFEX®), irinotecan (Camptosar®), L-asparaginase (ELSPAR®), leucovorin calcium, melphalan (Alkeran®), 6-mercaptopurine (Purinethol®), methotrexate (Folex®), mitoxantrone (Novantrone®), mylotarg, paclitaxel (Taxol®), phoenix (Yttrium90/MX-DTPA), pentostatin, polifeprosan 20 with carmustine implant (Gliadel®), tamoxifen citrate (Nolvadex®), teniposide (Vumon®), 6-thioguanine, thiotepa, tirapazamine (Tirazonet), topotecan hydrochloride for injection (Hycamptint), vinblastine (Velban®), vincristine (Oncovin®), and vinorelbine (Navelbine®). Exemplary alkylating agents include, without limitation, nitrogen mustards, ethylenimine derivatives, alkyl sulfonates, nitrosoureas and triazenes): uracil mustard (Aminouracil Mustard®, Chlorethaminacil®, Demethyldopan®, Desmethyldopan®, Haemanthamine®, Nordopan®, Uracil nitrogen Mustard®, Uracillost®, Uracilmostaza®, Uramustin®, Uramustine®), chlormethine (Mustargen®), cyclophosphamide (Cytoxan®, Neosar®, Clafen®, Endoxan®, Procytox®, Revimmune™), ifosfamide (Mitoxana®), melphalan (Alkeran®), Chlorambucil (Leukeran®), pipobroman (Amedel®, Vercyte®), triethylenemelamine (Hemel®, Hexalen®, Hexastat®), triethylenethiophosphoramine, Temozolomide (Temodar®), thiotepa (Thioplex®), busulfan (Busilvex®, Myleran®), carmustine (BiCNU®), lomustine (CeeNU®), streptozocin (Zanosar®), and Dacarbazine (DTIC-Dome®). Additional exemplary alkylating agents include, without limitation, Oxaliplatin (Eloxatin®); Temozolomide (Temodar® and Temodal®); Dactinomycin (also known as actinomycin-D, Cosmegen®); Melphalan (also known as L-PAM, L-sarcolysin, and phenylalanine mustard, Alkeran®); Altretamine (also known as hexamethylmelamine (HMM), Hexalen®); Carmustine (BiCNU®); Bendamustine (Treanda®); Busulfan (Busulfex® and Myleran®); Carboplatin (Paraplatin®); Lomustine (also known as CCNU, CeeNU®); Cisplatin (also known as CDDP, Platinol® and Platinol®-AQ); Chlorambucil (Leukeran®); Cyclophosphamide (Cytoxan® and Neosar®); Dacarbazine (also known as DTIC, DIC and imidazole carboxamide, DTIC-Dome®); Altretamine (also known as hexamethylmelamine (HMM), Hexalen®); Ifosfamide (Ifex®); Prednumustine; Procarbazine (Matulane®); Mechlorethamine (also known as nitrogen mustard, mustine and mechloroethamine hydrochloride, Mustargen®); Streptozocin (Zanosar®); Thiotepa (also known as thiophosphoamide, TESPA and TSPA, Thioplex®); Cyclophosphamide (Endoxan®, Cytoxan®, Neosar®, Procytox®, Revimmune®); and Bendamustine HCl (Treanda®). Exemplary mTOR inhibitors include, e.g., temsirolimus; ridaforolimus (formally known as deferolimus, (1R,2R,45)-4-[(2R)-2 [(1R,95,125,15R,16E,18R,19R,21R,235,24E,26E,28Z,305,325,35R)-1,18-dihydroxy-19,30-dimethoxy-15,17,21,23, 29,35-hexamethyl-2,3,10,14,20-pentaoxo-11,36-dioxa azatricyclo[30.3.1.04′9] hexatriaconta-16,24,26,28-tetraen-12-yl]propyl]-2-methoxycyclohexyl dimethylphosphinate, also known as AP23573 and MK8669, and described in PCT Publication No. WO 03/064383); everolimus (Afinitor® or RADOO1); rapamycin (AY22989, Sirolimus®); simapimod (CAS 164301-51-3); emsirolimus, (5-{2,4-Bis[(35,)-3-methylmorpholin-4-yl]pyrido[2,3-(i]pyrimidin-7-yl}-2-methoxyphenyl)methanol (AZD8055); 2-Amino-8-[iraw5,-4-(2-hydroxyethoxy)cyclohexyl]-6-(6-methoxy-3-pyridinyl)-4-methyl-pyrido[2,3-JJpyrimidin-7(8H)-one (PF04691502, CAS 1013101-36-4); and N2-[1,4-dioxo-4-[[4-(4-oxo-8-phenyl-4H-1-benzopyran-2-yl)morpholinium-4-yl]methoxy]butyl]-L-arginylglycyl-L-a-aspartylL-serine-, inner salt (SF1126, CAS 936487-67-1), and XL765. Exemplary immunomodulators include, e.g., afutuzumab (available from Roche®); pegfilgrastim (Neulasta®); lenalidomide (CC-5013, Revlimid®); thalidomide (Thalomid®), actimid (CC4047); and IRX-2 (mixture of human cytokines including interleukin 1, interleukin 2, and interferon γ, CAS 951209-71-5, available from IRX Therapeutics). Exemplary anthracyclines include, e.g., doxorubicin (Adriamycin® and Rubex®); bleomycin (Lenoxane®); daunorubicin (dauorubicin hydrochloride, daunomycin, and rubidomycin hydrochloride, Cerubidine®); daunorubicin liposomal (daunorubicin citrate liposome, DaunoXome®); mitoxantrone (DHAD, Novantrone®); epirubicin (Ellence™); idarubicin (Idamycin®, Idamycin PFS®); mitomycin C (Mutamycin®); geldanamycin; herbimycin; ravidomycin; and desacetylravidomycin. Exemplary vinca alkaloids include, e.g., vinorelbine tartrate (Navelbine®), Vincristine (Oncovin®), and Vindesine (Eldisine®)); vinblastine (also known as vinblastine sulfate, vincaleukoblastine and VLB, Alkaban-AQ® and Velban®); and vinorelbine (Navelbine®). Exemplary proteosome inhibitors include bortezomib (Velcade®); carfilzomib (PX-171-007, (5)-4-Methyl-N-((5)-1-(((5)-4-methyl-1-((R)-2-methyloxiran-2-yl)-1-oxopentan-2-yl)amino)-1-oxo-3-phenylpropan-2-yl)-2-((5,)-2-(2-morpholinoacetamido)-4-phenylbutanamido)-pentanamide); marizomib (NPT0052); ixazomib citrate (MLN-9708); delanzomib (CEP-18770); and O-Methyl-N-[(2-methyl-5-thiazolyl)carbonyl]-L-seryl-O-methyl-N-[(11S′)-2-[(2R)-2-methyl-2-oxiranyl]-2-oxo-1-(phenylmethyl)ethyl]-L-serinamide (ONX-0912).

One of skill in the art can readily identify a chemotherapeutic agent of use with methods and compositions describe herein (e.g. see Physicians' Cancer Chemotherapy Drug Manual 2014, Edward Chu, Vincent T. DeVita Jr., Jones & Bartlett Learning; Principles of Cancer Therapy, Chapter 85 in Harrison's Principles of Internal Medicine, 18th edition; Therapeutic Targeting of Cancer Cells: Era of Molecularly Targeted Agents and Cancer Pharmacology, Chs. 28-29 in Abeloff s Clinical Oncology, 2013 Elsevier; and Fischer D S (ed): The Cancer Chemotherapy Handbook, 4th ed. St. Louis, Mosby-Year Book, 2003).

In accordance with one embodiment, the subject is administered a radiation therapy in combination with agonist of δ-opioid receptor, SNC-80, or Donepezil as described herein. Radiation therapy, according to the invention disclosed herein, encompasses both non-invasive (external) and invasive (internal) radiation therapies. In an external radiation therapy, treatment is affected by radiation sources outside the body, whereas in an invasive radiation therapy treatment is affected by radiation sources planted inside the body. The representative diseases treated by non-invasive or invasive radiation therapy include, for example, cancer, rheumatoid arthritis, angioplasty, or restenosis.

In accordance with one embodiment, the subject is administered a chemo-radiation therapy, e.g., a combination of a chemotherapy and radiation therapy, in combination with agonist of δ-opioid receptor, SNC-80, or Donepezil as described herein.

In one embodiment, administering is systemic or local administration, e.g., injection, diffusion, or perfusion of a cell, tissue or organ.

In one embodiment, the agonist, SNC-80, or Donepezil reduces cell death of non-cancer cells during the anti-cancer treatment. In one embodiment, the agonist, SNC-80, or Donepezil reduces cell death of non-cancer cells during the anti-cancer treatment by at least 5, 10, 15, 20, 25, 30, 35, 40,45, 50, 55, 60, 65, 70, 75, 80, 85, 90, 95, 99% or more as compared to a sample not contacted by an agonist, SNC-80, or Donepezil described herein. Cell death and/or degradation in a cell, tissue or organ can be assessed by one skilled in the art using standard techniques on, e.g., a tissue biopsy following treatment.

Another aspect herein provides a method of treating a hematological neoplastic disease comprising harvesting bone marrow from a subject having such a disease, contacting harvested bone marrow or a cellular fraction thereof with an agonist of the δ-opioid receptor and with one or more anti-cancer therapeutics at a dose sufficient to kill neoplastic cells, treating the subject with chemotherapy or radiation sufficient to kill remaining bone marrow hematologic stem cells, and then administering the contacted bone marrow or cellular fraction to the subject.

Another aspect herein provides a method of treating a hematological neoplastic disease comprising harvesting bone marrow from a subject having such a disease, contacting harvested bone marrow or a cellular fraction thereof with SNC-80 and with one or more anti-cancer therapeutics at a dose sufficient to kill neoplastic cells, treating the subject with chemotherapy or radiation sufficient to kill remaining bone marrow hematologic stem cells, and then administering the contacted bone marrow or cellular fraction to the subject.

Another aspect herein provides a method of treating a hematological neoplastic disease comprising harvesting bone marrow from a subject having such a disease, contacting harvested bone marrow or a cellular fraction thereof with an and with one or more anti-cancer therapeutics at a dose sufficient to kill neoplastic cells, treating the subject with chemotherapy or radiation sufficient to kill remaining bone marrow hematoligic stem cells, and then administering the contacted bone marrow or cellular fraction to the subject.

Another aspect herein provides a method of treating a hematological neoplastic disease comprising harvesting bone marrow from a subject having such a disease, contacting harvested bone marrow or a cellular fraction thereof with Donepezil and with one or more anti-cancer therapeutics at a dose sufficient to kill neoplastic cells, treating the subject with chemotherapy or radiation sufficient to kill remaining bone marrow hematologic stem cells, and then administering the contacted bone marrow or cellular fraction to the subject.

In one embodiment, treatment with an agonist of δ-opioid receptor, SNC-80, or Donepezil protects non-neoplastic cells from killing by the one or more anti-cancer therapeutics. In one embodiment, the agonist, SNC-80, or Donepezil reduces non-neoplastic cells from killing by the one or more anti-cancer therapeutics by at least 5, 10, 15, 20, 25, 30, 35, 40,45, 50, 55, 60, 65, 70, 75, 80, 85, 90, 95, 99% or more as compared to a sample not contacted by an agonist, SNC-80, or Donepezil as described herein.

In one embodiment, the method further comprises contacting the cell, tissue or organ with at least a second biostatic composition or compound. In one embodiment, the second compound is selected from the group consisting of hydrogen sulfide, adenosine monophosphate (AMP), a neuropeptide, deferoxamine, and a prolyl hydroxylase inhibitor.

### Administration

In some embodiments, the method of preserving healthy cells in a subject undergoing a cancer treatment comprises administering to the subject receiving or to receive an anti-cancer therapy an agonist of δ-opioid receptor, SNC-80, or Donepezil. Subjects having a cancer can be identified by a physician using current methods of diagnosing a cancer. Symptoms, and/or complications of the cancer, which characterize this disease and aid in diagnosis are well known in the art and include but are not limited to, fatigue, weight loss, bone pain, swollen or painful lymph nodes, and headaches. Tests that may aid in a diagnosis of, e.g. the cancer, include but are not limited to, punch or excision biopsy, and non-invasive imaging (e.g., Magnetic Resonance Imaging, or Computerized Tomography scan), and are known in the art for a given condition. A family history for a condition, or exposure to risk factors for a cancer can also aid in determining if a subject is likely to have the condition or in making a diagnosis of the cancer.

An agonist of δ-opioid receptor, SNC-80, or Donepezil as described herein can be administered to a subject having or diagnosed as having a cancer and who is receiving an anti-cancer therapy.

An agonist of δ-opioid receptor, SNC-80, or Donepezil as described herein can be administered to a subject having or diagnosed in need of preserving tissue or an organ. An isolated tissue or an organ can be directly contacted with an agonist of δ-opioid receptor, SNC-80, or Donepezil as described herein.

In one embodiment, the agonist of δ-opioid receptor, SNC-80, or Donepezil is administered systemically or locally, e.g., diffusion, injection, perfusion to a cell, tissue or organ. In one embodiment, contacting is submerging an isolated cell, tissue or organ is in a composition comprising the agonist of δ-opioid receptor, SNC-80, or Donepezil. In one embodiment, the agonist, SNC-80, or Donepezil is administered intravenously.

In one embodiment, the agonist, SNC-80, or Donepezil is administered or contacts the cell, tissue or organ once. In an alternate embodiment, the agonist, SNC-80, or Donepezil is administered or contacts the cell, tissue or organ at least twice, for example, at least once per hour, day, week or more. In one embodiment, the dosage of each contact is the same. Alternatively, the dosage of an agonist, SNC-80, or Donepezil can vary between administerations or contacts. For example, the initial administering or contacting can comprise a high dose of the agonist of δ-opioid receptor, SNC-80, or Donepezil, followed by at least one subsequent lower dose of the agonist of δ-opioid receptor, SNC-80, or Donepezil, respectively.

The term “effective amount” as used herein refers to the amount of an agonist, SNC-80, or Donepezil needed to induce biostasis of a cell, tissue or organ, or preserve healthy cells in a subject undergoing a cancer treatment. The term “therapeutically effective amount” can refer to an amount of an agonist, SNC-80, or Donepezil that is sufficient to induce biostasis of a cell, tissue or organ following contact. The term “therapeutically effective amount” can refer to an amount of an agonist, SNC-80, or Donepezil that is sufficient preserving healthy cells in a subject undergoing a cancer treatment when administered to a typical subject. Thus, it is not generally practicable to specify an exact “effective amount”. However, for any given case, an appropriate “effective amount” can be determined by one of ordinary skill in the art using only routine experimentation. It is specifically contemplated that the “therapeutically effective amount” is not so high that it induces biostasis that is not reversible, e.g., a cell, tissue or organ that is unable to recover from biostasis. For example, for transplant, the “therapeutically effective amount” would be one that would induce biostasis and allow for recovery (e.g., return of function of the cell, tissue or organ following induced biostasis) prior to, or following transplant.

Effective amounts, toxicity, and therapeutic efficacy can be evaluated by standard pharmaceutical procedures in cell cultures or experimental animals. The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, a dose can be formulated in animal models to achieve a circulating plasma concentration range that includes the IC50 (i.e., the concentration of the agonist, SNC-80, or Donepezil, which achieves a half-maximal inhibition of symptoms) as determined in cell culture, or in an appropriate animal model. Levels in plasma can be measured, for example, by high performance liquid chromatography. The effects of any particular dosage can be monitored by a suitable bioassay, e.g., noninvasive imaging, among others. The dosage can be determined by a physician and adjusted, as necessary, to suit observed effects of the treatment. Where high doses of δ-opioid receptor agonists such as SNC-80 are known to induce serious side effects including convulsions, localized dosing may be preferable to systemic dosing.

### Combination Treatment

While it is contemplated herein that the preservation of healthy cells in a subject undergoing a cancer treatment can be accomplished by administering only an agonist of δ-opioid receptor, SNC-80, or Donepezil to a subject co-administered an anti-cancer treatment to preserve healthy cells in a subject in one embodiment, the agonist is administered with at least a second biostatic agent or under a specific condition, e.g., hypoxia, osmotic stress, physiological stress, burn injury, blast injury, trauma, radiation, chemical exposure, toxin exposure and cooling or freezing condition.

Administered “in combination,” as used herein, means that two (or more) different treatments, e.g., the agonist, SNC-80, or Donepezil and anti-cancer therapy, are delivered to the subject during the course of the subject's affliction with the disorder, e.g., the two or more treatments are delivered after the subject has been diagnosed with the disorder or disease (for example, cancer) and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered. The agents described herein and the at least one biostatic agent can be administered simultaneously, in the same or in separate compositions, or sequentially. For sequential administration, the agonist, SNC-80, or Donepezil described herein can be administered first, and the at least one biostatic agent can be administered second, or the order of administration can be reversed. The agonist, SNC-80, or Donepezil and/or other at least one biostatic agent, procedures or modalities can be administered during periods of active disorder, or during a period of remission or less active disease. The agonist can be administered before another treatment, concurrently with the treatment, post-treatment, or during remission of the disorder.

### Dosage

Where a 8-opioid receptor agonist is contacted with a cell, tissue or organ for transplantation, the concentration of the agonist will depend upon the identity of that agonist. The agonist is to be administered at a dose sufficient to alter the function of at least one ion channel selected from the group consisting of EAAT1 ion channel and NCX1 ion channel By way of non-limiting example, SNC-80 can be used in a range of 50 to 1000 micromolar (μM) concentration, e.g., at least 50 μM, 60 μM, 70 μM, 80 μM, 90 μM, 100 μM, 120 μM, 140 μM, 160 μM, 180 μM, 200 μM, 220 μM, 240 μM, 260 μM, 280 μM, 300 μM, 320 μM, 340 μM, 360 μM, 380 μM, 400 μM, 420 μM, 440 μM, 460 μM, 480 μM, 500 μM, 520 μM, 540 μM, 560 μM, 580 μM, 600 μM, 620 μM, 640 μM, 680 μM, 700 μM, 720 μM, 740 μM, 760 μM, 780 μM, 800 μM, 820 μM, 840 μM, 860 μM, 880 μM, 900 μM, 920 μM, 940 μM, 960 μM, 980 μM or 1000 μM (1 mM). Initial effective concentrations for other 8-opioid receptor agonists can be gauged by considering the potency of the other agonist relative to SNC-80.

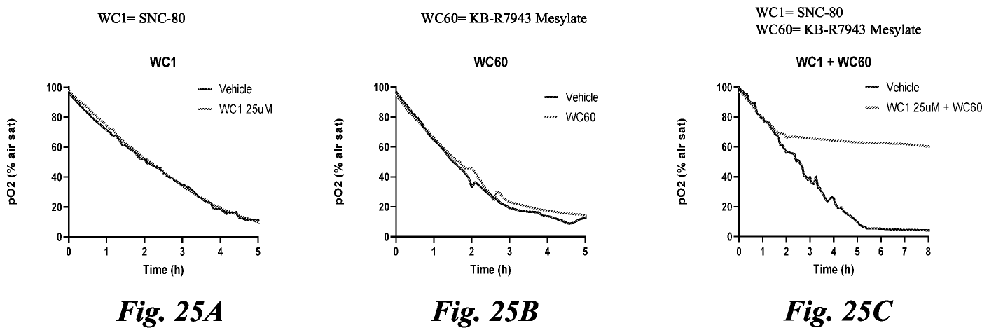

Alternatively, in one embodiment, SNC-80 is be administered at a lower dose, e.g., 25 μM concentration, when administered in combination with at least a second agent (see, e.g., FIGS. 25A-25C). In one embodiment, SNC-80 is administered at a dose that is not sufficient to induce biostasis alone, e.g., 25 μM concentration, when administered in combination with at least a second agent (see, e.g., FIGS. 25A-25C).

In one embodiment, SNC-80 is administered at a range of 50 to 900 μM concentration, 50 to 800 μM concentration, 50 to 100 μM concentration, 50 to 150 μM concentration 50 to 700 μM concentration, 50 to 600 μM concentration, 50 to 500 μM concentration, 50 to 400 μM concentration, 50 to 300 μM concentration, 50 to 200 μM concentration, 50 to 1000 μM concentration, 50 to 1000 μM concentration, 50 to 1000 μM concentration, 500 to 1000 μM concentration, 600 to 1000 μM concentration, 700 to 1000 μM concentration, 800 to 1000 μM concentration, 900 to 1000 μM concentration, 200 to 800 μM concentration, 200 to 600 μM concentration, 200 to 500 μM concentration, 300 to 800 μM concentration, 300 to 700 μM concentration, 300 to 600 μM concentration, 250 to 750 μM concentration, 250 to 500 μM concentration, 400 to 700 μM concentration, 400 to 600 μM or concentration.

In one embodiment, Donepezil is used at a concentration at or greater than 25 μM, e.g., at least 25 μM, 30 μM, 40 μM, 50 μM, 60 μM, 70 μM, 80 μM, 90 μM, 100 μM, 110 μM, 120 μM, 130 μM, 140 μM, 150 μM, 160 μM, 170 μM, 180 μM, 190 μM, 200 μM, 210 μM, 220 μM, 230 μM, 240 μM, 250 μM, 260 μM, 270 μM, 280 μM, 290 μM, 300 μM, 400 μM, 500 μM, 600 μM, 700 μM, 800 μM, 900 μM, 1 mM, 2 mM, 3 mM, 4 mM, or more. In one embodiment, Donepezil is used at a concentration of 25 μM. In one embodiment, Donepezil is used at a concentration of 50 μM. In one embodiment, Donepezil is used at a concentration range from 25-50 μM.

In one embodiment, the polyol, e.g., kestose or erlose, is used at a dose of 50 mM. In one embodiment, the polyol, e.g., kestose or erlose, is used in a range of 500 μM-500 mM, e.g., at least 500 μM, 600 μM, 700 μM, 800 μM, 900 μM, 1 mM, 5 mM, 10 mM, 20 mM, 30 mM, 40 mM, 50 mM, 60 mM, 70 mM, 80 mM, 90 mM, 100 mM, 110 mM, 120 mM, 130 mM, 140 mM, 150 mM, 160 mM, 170 mM, 180 mM, 190 mM, 200 mM, 210 mM, 220 mM, 230 mM, 240 mM, 250 mM, 260 mM, 270 mM, 280 mM, 290 mM, 300 mM, 310 mM, 320 mM, 330 mM, 340 mM, 350 mM, 360 mM, 370 mM, 380 mM, 390 mM, 400 mM, 410 mM, 420 mM, 430 mM, 440 mM, 450 mM, 460 mM, 470 mM, 480 mM, 490 mM, 500 mM.

In one embodiment, KB-R7943 Mesylate is used in a range 3.5 μM-100 μM when exposure time is greater than 2 hours, e.g., at least 3.5 μM, 5 μM, 10 μM, 15 μM, 20 μM, 25 μM, 30 μM, 40 μM, 50 μM, 60 μM, 70 μM, 80 μM, 90 μM, 100 μM. In one embodiment, KB-R7943 Mesylate is used in a range 3.5 μM-1 mM when exposure time is less than 2 hours, e.g., at least 3.5 μM, 5 μM, 10 μM, 15 μM, 20 μM, 25 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM.

In one embodiment, KB-R7943 Mesylate is be administered at a lower dose, e.g., 35 μM concentration, when administered in combination with at least a second agent (see, e.g., FIGS. 25A-25C). In one embodiment, KB-R7943 Mesylate is administered at a dose that is not sufficient to induce biostasis alone, e.g., 35 μM concentration, when administered in combination with at least a second agent (see, e.g., FIGS. 25A-25C).

In one embodiment, Aprindine, is used at a dose of 25 μM. In one embodiment, Aprindine, is used at a dose of 30 μM, 35 μM, 40 μM, 45 μM, 50 μM, 55 μM, 60 μM, 65 μM, 70 μM, 75 μM, or more. In one embodiment, Aprindine, is used at a dose range of 25 μM-50 μM, 25 μM-35 μM, 25 μM-45 μM, 25 μM-55 μM, 25 μM-65 μM, 25 μM-75 μM, 35 μM-75 μM, 35 μM-65 μM, 35 μM-55 μM, 50 μM-75 μM, 50 μM-65 μM.

In certain embodiments, when an agent or agonist is administered in combination with at least a second agent, the dose of the agent or agonist can be administered at a lower dose than if it is administered alone.

In certain embodiments, when an agent or agonist is administered in combination with deuterium oxide, the dose of the agent or agonist can be administered at a lower dose than if it is administered alone. For example, in one embodiment, Donepezil is used at a concentration of 40 μM, 20 μM, or 10 μM when combined with 50% deuterium oxide. In one embodiment, Donepezil is used at a concentration of 40 μM, 20 μM, or 10 μM when combined with 25% deuterium oxide.

Dosages described herein can be administered at least once an hour, at least once a day, at least once a week, at least once a month, at least once a year, or longer

In those instances, where an agonist of δ-opioid receptor, SNC-80 or Donepezil is administered to a patient, e.g., as part of a cancer treatment regimen, the dosage can be determined by a physician and adjusted, as necessary, to suit observed effects of the treatment. It is specifically contemplated herein that the dosage of an agent described herein dependent if administration is to a subject, e.g., as part of a cancer treatment regimen, or to an isolated cell, tissue or organ. With respect to duration and frequency of treatment, it is typical for skilled clinicians to monitor subjects in order to determine when the treatment is providing therapeutic benefit, and to determine whether to administer further doses, discontinue treatment, resume treatment, or make other alterations to the treatment regimen. The dosage should not be so large as to cause adverse side effects, such as cytotoxic effects or convulsions. The dosage can also be adjusted by the individual physician in the event of any complication.

“Unit dosage form” as the term is used herein refers to a dosage for suitable one administration. By way of example a unit dosage form can be an amount of therapeutic disposed in a delivery device, e.g., a syringe or intravenous drip bag. In one embodiment, a unit dosage form is administered in a single administration. In another, embodiment more than one unit dosage form can be administered simultaneously.

The dosage range depends upon the potency, and includes amounts large enough to produce the desired effect, e.g., prevent killing of healthy cells caused by an anti-cancer therapy. Generally, the dosage will vary with the age, sex, and condition of the patient. Typically, the dosage will range from 0.001 mg/kg body weight to 5 g/kg body weight. In some embodiments, the dosage range is from 0.001 mg/kg body weight to 1 g/kg body weight, from 0.001 mg/kg body weight to 0.5 g/kg body weight, from 0.001 mg/kg body weight to 0.1 g/kg body weight, from 0.001 mg/kg body weight to 50 mg/kg body weight, from 0.001 mg/kg body weight to 25 mg/kg body weight, from 0.001 mg/kg body weight to 10 mg/kg body weight, from 0.001 mg/kg body weight to 5 mg/kg body weight, from 0.001 mg/kg body weight to 1 mg/kg body weight, from 0.001 mg/kg body weight to 0.1 mg/kg body weight, from 0.001 mg/kg body weight to 0.005 mg/kg body weight. Alternatively, in some embodiments the dosage range is from 0.1 g/kg body weight to 5 g/kg body weight, from 0.5 g/kg body weight to 5 g/kg body weight, from 1 g/kg body weight to 5 g/kg body weight, from 1.5 g/kg body weight to 5 g/kg body weight, from 2 g/kg body weight to 5 g/kg body weight, from 2.5 g/kg body weight to 5 g/kg body weight, from 3 g/kg body weight to 5 g/kg body weight, from 3.5 g/kg body weight to 5 g/kg body weight, from 4 g/kg body weight to 5 g/kg body weight, from 4.5 g/kg body weight to 5 g/kg body weight, from 4.8 g/kg body weight to 5 g/kg body weight. In some embodiments of any of the aspects, the dose range is from 1 μg/kg body weight to 20 μg/kg body weight. Alternatively, the dose range will be titrated to maintain serum levels between 1 μg/mL and 20 μg/mL. In some embodiments, the dosage range is from 1 μg/mL to 15 μg/mL, from 1 μg/mL to 10 μg/mL, from 1 μg/mL to 5 μg/mL, from 1 μg/mL to 2.5 μg/mL, from 2.5 μg/mL to 20 μg/mL, from 5 μg/mL to 20 μg/mL, from 10 μg/mL to 20 μg/mL, from 15 μg/mL to 20 μg/mL, from 10 μg/mL to 5 μg/mL, from 5 μg/mL to 15 μg/mL, from 5 μg/mL to 10 μg/mL, from 2.5 μg/mL to 10 μg/mL, or from 2.5 μg/mL to 15 μg/mL.

Modes of administration can include, for example intravenous (i.v.) injection or infusion. The compositions described herein can be also be administered to a patient transarterially, intratumorally, or intranodally. In some embodiments, the agonist, SNC-80, or Donepezil can be injected directly into a tumor or lymph node, or, for example, into adjacent healthy tissue. In one embodiment, the agonist, SNC-80, or Donepezil described herein is administered into a body cavity or body fluid (e.g., ascites, pleural fluid, peritoneal fluid, or cerebrospinal fluid).

### Parenteral Dosage Forms

Where appropriate, parenteral dosage forms of a 8-opioid receptor agonist, SNC-80, or Donepezil as described herein can be administered to a subject by various routes, including, but not limited to, epidural, intracerebral, intracerebroventricular, epicutaneous, nasal administration, intraarterial, intraarticular, intracardiac, intracavernous injection, intradermal, intralesional, intramuscular, intraocular, intraosseous infusion, intraperitoneal, intrathecal, intrauterine, intravaginal administration, intravenous, intravesical, intravitreal, subcutaneous, transdermal, perivascular administration, or transmucosal. Since administration of parenteral dosage forms typically bypasses the patient's natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, controlled-release parenteral dosage forms, and emulsions.

Suitable vehicles that can be used to provide parenteral dosage forms of the disclosure are well known to those skilled in the art. Examples include, without limitation: sterile water; water for injection USP; saline solution; glucose solution; aqueous vehicles such as but not limited to, sodium chloride injection, Ringer's injection, dextrose Injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate.

### Efficacy

The efficacy of an agonist of delta-opioid receptor, SNC-80, or Donepezil, e.g., for inducing biostasis or preventing killing of healthy cells during an anti-cancer therapy, described herein can be determined by the skilled clinician. However, a treatment is considered “effective treatment,” as the term is used herein, if one or more of the signs of a biostasis are observed following treatment according to the methods described herein. Efficacy can be assessed, for example, by measuring a marker or indicator and/or the incidence of biostasis according to the methods described herein or any other measurable parameter appropriate (e.g., a reduction in oxygen uptake by the contacted cell, tissue or organ. Efficacy of biostasis can be assessed by its ability to preserve an isolated cell, tissue or organ, e.g., by preventing cellular death in the cell, tissue or organ for an longer period of time as compared to an untreated cell, tissue or organ. Efficacy of biostasis of an organ or tissue to be transplanted can be assessed be determining if the preserved cell, tissue or organ results in, e.g., an increase in time between tissue harvest and tissue transplant without death of the tissue. Increased as that term is defined herein, for example, by at least 1 hour, at least 2 hours, at least 3 hours, etc.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The invention can be further described in the following numbered paragrahs.
1. A method of inducing biostasis in a cell, tissue or organ, the method comprising contacting the cell, tissue or organ in need of preservation with an agent that alters the function of at least one ion channel selected from the group consisting of EAAT1 ion channel and NCX1 ion channel, wherein the contacted cell, tissue or organ exhibits biostasis.
2. A method of preserving viability or function of a cell, tissue or organ, the method comprising contacting the cell, tissue or organ in need of such preservation with an agent that alters the function of at least one ion channel selected from the group consisting of EAAT1 ion channel and NCX1 ion channel
3. A method of cell, tissue or organ transplant, the method comprising contacting a donor cell, tissue or organ in situ or ex vivo with an agent that alters the function of at least one ion channel selected from the group consisting of EAAT1 ion channel and NCX1 ion channel
4. A method of preparing a cell, tissue or organ for transplant, the method comprising contacting a donor cell, tissue or organ with an agent that alters the function of at least one ion channel selected from the group consisting of EAAT1 ion channel and NCX1 ion channel
5. The method of any preceding paragraphs, wherein the agent further activates the δ-opioid receptor following contact.
6. The method of any preceding paragraphs, wherein the agent does not activate the δ-opioid receptor following contact.
7. A method of inducing biostasis in a cell, tissue or organ, the method comprising contacting the cell, tissue or organ in need of preservation with an agonist for the δ-opioid receptor, wherein the contacted cell, tissue or organ exhibits biostasis, wherein the agonist is administered at a dose sufficient to alter the function of at least one ion channel selected from the group consisting of EAAT1 ion channel and NCX1 ion channel
8. A method of preserving viability or function of a cell, tissue or organ, the method comprising contacting the cell, tissue or organ in need of such preservation with an agonist of the δ-opioid receptor, wherein the agonist is administered at a dose sufficient to alter the function of at least one ion channel selected from the group consisting of EAAT1 ion channel and NCX1 ion channel
9. A method of cell, tissue or organ transplant, the method comprising contacting a donor cell, tissue or organ in situ or ex vivo with an agonist of the δ-opioid receptor, wherein the agonist is administered at a dose sufficient to alter the function of at least one ion channel selected from the group consisting of EAAT1 ion channel and NCX1 ion channel
10. A method of preparing a cell, tissue or organ for transplant, the method comprising contacting a donor cell, tissue or organ with an agonist of the δ-opioid receptor, wherein the agonist is administered at a dose sufficient to alter the function of at least one ion channel selected from the group consisting of EAAT1 ion channel and NCX1 ion channel
11. The method of any preceding paragraphs, wherein altering the function is inhibiting the function.
12. The method of any preceding paragraphs, wherein altering the function is slowing the function.
13. The method of any preceding paragraphs, wherein altering the function is activating the function.
14. The method of any preceding paragraphs, wherein the tissue is an endoderm tissue, a mesoderm tissue, or an ectoderm tissue.
15. The method of any preceding paragraphs, wherein the tissue is selected from the group consisting of cornea, bone, tendon, pancreas islet, heart valve, nerve, vascular, deep tissue flap, fat tissue, muscle, and vein.
16. The method of any preceding paragraphs, wherein the organ is selected from the group consisting of intestine, stomach, heart, kidney, bladder, pancreas, liver, lung, brain, skin, uterus, digit, and limb.
17. The method of any preceding paragraphs, wherein the contacting suppresses the metabolism or induces biostasis of the cell, tissue or organ.
18. The method of any preceding paragraphs, wherein the agent is SNC-80 or donepezil.
19. The method of any preceding paragraphs, wherein the agonist is SNC-80.
20. The method of any preceding paragraphs, wherein the agent is a derivative, analog, or variant of SNC-80 that alters the function of at least one ion channel selected from the group consisting of EAAT1 ion channel and NCX1 ion channel
21. The method of any preceding paragraphs, wherein the agent is a derivative, analog, or variant of donepezil that alters the function of at least one ion channel selected from the group consisting of EAAT1 ion channel and NCX1 ion channel
22. The method of any preceding paragraphs, wherein the agonist is a derivative, analog, or variant of SNC-80 that activates signaling by the δ-opioid receptor and alters the function of at least one ion channel selected from the group consisting of EAAT1 ion channel and NCX1 ion channel
23. The method of any preceding paragraphs, further comprising contacting with at least a second agent that alters the function of at least one ion channel selected from the group consisting of EAAT1 ion channel and NCX1 ion channel
24. The method of any preceding paragraphs, wherein the agonist or agent and the at least second agent are contacted at substantially the same time.
25. The method of any preceding paragraphs, wherein the agonist or agent and the at least second agent are contacted at different times.
26. The method of any preceding paragraphs, wherein the at least second agent is an inhibitor of the NCX1 ion channel
27. The method of any preceding paragraphs, wherein the inhibitor is KB-R7943 mesylate.
28. The method of any preceding paragraphs, wherein the agent or agonist is comprised in a vehicle that is deuterium oxide.
29. The method of any preceding paragraphs, wherein the contacting is short-term or long-term.
30. The method of any preceding paragraphs, wherein the contacting is a single contact, or reoccurring contacting.
31. The method of any preceding paragraphs, wherein one or more genes listed in Table 1, or gene products thereof, are modulated by agent or agonist following contacting.
32. The method of any preceding paragraphs, wherein the contacting is performed for at least 1 hour, 2 hours, 3 hours, 4 hours, 5 hours, 6 hours, 10 hours, 18 hours, 24 hours, 36 hours 48, hour, 96 hours or more.
33. The method of any preceding paragraphs, wherein contacting is performed via diffusion, perfusion, injection, immersion, or delivery via air.
34. The method of any preceding paragraphs wherein the diffusion, perfusion, injection, immersion, or delivery via air is performed in vivo or ex vivo.
35. The method of any preceding paragraphs, wherein contacting is performed via direct introduction to the cell, tissue or organ.
36. The method of any preceding paragraphs, wherein the cell, tissue or organ is contacted prior to removal from the donor for a transplant in a recipient.
37. The method of any preceding paragraphs, wherein the cell, tissue or organ is preserved contacted following removal from the donor, and prior to a transplant in a recipient.
38. The method of any preceding paragraphs, wherein the cell, tissue or organ is contacted following an injury to the cell, tissue or organ.
39. The method of any preceding paragraphs, wherein the cell, tissue or organ is contacted prior to a surgical procedure.
40. The method of any preceding paragraphs, wherein the cell, tissue or organ is contacted during a therapeutic treatment.
41. The method of any preceding paragraphs, wherein the therapeutic treatment is an anti-cancer treatment.
42. The method of any preceding paragraphs, wherein the anti-cancer treatment is radiation, chemotherapy, immunotherapy, CAR-T cell therapy, or other cellular therapy.
43. The method of any preceding paragraphs, wherein the contacting permits treatment with a higher dose of anti-cancer treatment relative to treatment in the absence of the contacting.
44. The method of any preceding paragraphs, further comprising contacting the cell, tissue or organ with at least a second, biostatic compound.
45. The method of any preceding paragraphs, wherein the at least a second compound is selected from the group consisting of hydrogen sulfide, nitrogen, argon, Oligomycin A, rotenone, 2-deoxyglucose, adenosine monophosphate (AMP), a neuropeptide, deferoxamine, and a prolyl hydroxylase inhibitor.
46. The method of any preceding paragraphs, wherein the cell, tissue or organ are contacted with the agonist and the at least a second compound at substantially the same time.
47. The method of any preceding paragraphs, wherein the cell, tissue or organ are contacted with the agonist and the at least a second compound at different times.
48. The method of any preceding paragraphs, wherein the cell, tissue or organ is contacted with the agonist under a condition selected from the group consisting of hypoxia, osmotic stress, physiological stress, burn injury, blast injury, trauma, radiation, chemical exposure, toxin exposure and cooling or freezing condition.
49. The method of any preceding paragraphs, wherein the contacting comprises induction of biostasis that is reversed following withdrawal of the agonist and/or administration of an opioid antagonist.
50. The method of any preceding paragraphs, wherein the contacting does not induce hypothermia.
51. The method of any preceding paragraphs, wherein the agonist is not contacted in combination with a local anesthetic, an anti-arrhythmic, citrate, or magnesium.
52. A method of inducing biostasis in a cell, tissue or organ, the method comprising contacting the cell, tissue or organ in need of preservation with at least two agents that alter the function of at least one ion channel selected from the group consisting of EAAT1 ion channel and NCX1 ion channel, wherein the contacted cell, tissue or organ exhibits biostasis.
53. A method of preserving viability or function of a cell, tissue or organ, the method comprising contacting the cell, tissue or organ in need of such preservation with at least two agents that alter the function of at least one ion channel selected from the group consisting of EAAT1 ion channel and NCX1 ion channel
54. A method of cell, tissue or organ transplant, the method comprising contacting a donor cell, tissue or organ in situ or ex vivo with at least two agents that alter the function of at least one ion channel selected from the group consisting of EAAT1 ion channel and NCX1 ion channel
55. A method of preparing a cell, tissue or organ for transplant, the method comprising contacting a donor cell, tissue or organ with at least two agents that alter the function of at least one ion channel selected from the group consisting of EAAT1 ion channel and NCX1 ion channel
56. A method of inducing biostasis in a cell, tissue or organ, the method comprising contacting the cell, tissue or organ in need of preservation with an agonist for the δ-opioid receptor and an agent that alters the function of at least one ion channel selected from the group consisting of EAAT1 ion channel and NCX1 ion channel, wherein the contacted cell, tissue or organ exhibits biostasis, wherein the agonist is administered at a dose sufficient to alter the function of at least one ion channel selected from the group consisting of EAAT1 ion channel and NCX1 ion channel
57. A method of preserving viability or function of a cell, tissue or organ, the method comprising contacting the cell, tissue or organ in need of such preservation with an agonist of the δ-opioid receptor and an agent that alters the function of at least one ion channel selected from the group consisting of EAAT1 ion channel and NCX1 ion channel, wherein the agonist is administered at a dose sufficient to alter the function of at least one ion channel selected from the group consisting of EAAT1 ion channel and NCX1 ion channel
58. A method of cell, tissue or organ transplant, the method comprising contacting a donor cell, tissue or organ in situ or ex vivo with an agonist of the δ-opioid receptor and an agent that alters the function of at least one ion channel selected from the group consisting of EAAT1 ion channel and NCX1 ion channel, wherein the agonist is administered at a dose sufficient to alter the function of at least one ion channel selected from the group consisting of EAAT1 ion channel and NCX1 ion channel
59. A method of preparing a cell, tissue or organ for transplant, the method comprising contacting a donor cell, tissue or organ with an agonist of the δ-opioid receptor and an agent that alters the function of at least one ion channel selected from the group consisting of EAAT1 ion channel and NCX1 ion channel, wherein the agonist is administered at a dose sufficient to alter the function of at least one ion channel selected from the group consisting of EAAT1 ion channel and NCX1 ion channel
60. The method of any preceding paragraphs, wherein at least one of that at least two agents are contacted at a sub-biostasis dose.
61. The method of any preceding paragraphs, wherein the agonist or agent are contacted at a sub-biostasis dose.
62. A composition comprising at least two agents that alter the function of at least one ion channel selected from the group consisting of EAAT1 ion channel and NCX1 ion channel
63. A composition comprising an agonist of the δ-opioid receptor and an agent that alters the function of at least one ion channel selected from the group consisting of EAAT1 ion channel and NCX1 ion channel
64. The method of any preceding paragraphs, further comprising deuterium oxide.
65. A composition comprising deuterium oxide and an agent that alter the function of at least one ion channel selected from the group consisting of EAAT1 ion channel and NCX1 ion channel
66. A composition comprising deuterium oxide and an agonist of the δ-opioid receptor.
67. A method of preserving healthy cells in a subject undergoing a cancer treatment, the method comprising administering to the subject receiving or to receive an anti-cancer therapy
   a. an agonist of the δ-opioid receptor, wherein the agonist is administered at a dose sufficient to alter the function of at least one ion channel selected from the group consisting of EAAT1 ion channel and NCX1 ion channel; or
   b. an agent that alters the function of at least one ion channel selected from the group consisting of EAAT1 ion channel and NCX1 ion channel
68. The method of any preceding paragraphs, wherein the administering is performed prior to, at substantially the same time, and/or after receiving an anti-cancer therapy.
69. The method of any preceding paragraphs, wherein the agonist is SNC-80.
70. The method of any preceding paragraphs, wherein the agonist is a derivative, analog, or variant of SNC-80.
71. The method of any preceding paragraphs, wherein the agent is SNC-80 or donepezil.
72. The method of any preceding paragraphs, wherein the agent is a derivative, analog, or variant of SNC-80 or donepezil.
73. The method of any preceding paragraphs, wherein the anti-cancer treatment is radiation or chemotherapy.
74. The method of any preceding paragraphs, wherein the anti-cancer treatment is high dose or high exposure treatment.
75. The method of any preceding paragraphs, wherein administering is systemic or local administration.
76. The method of any preceding paragraphs, wherein local administration is perfusion.
77. The method of any preceding paragraphs, wherein the agonist prevents or reduces cell death of non-cancer cells during the anti-cancer treatment.
78. A method of treating a hematological neoplastic disease, the method comprising harvesting bone marrow from a subject having such a disease, contacting harvested bone marrow or a cellular fraction thereof with
   a. an agonist of the δ-opioid receptor, wherein the agonist is administered at a dose sufficient to alter the function of at least one ion channel selected from the group consisting of EAAT1 ion channel and NCX1 ion channel; or
   b. an agent that alters the function of at least one ion channel selected from the group consisting of EAAT1 ion channel and NCX1 ion channel; and with one or more anti-cancer therapeutics at a dose sufficient to kill neoplastic cells, treating the subject with chemotherapy or radiation sufficient to kill remaining bone marrow hematologic stem cells, and then administering the contacted bone marrow or cellular fraction to the subject.
79. The method of any preceding paragraphs, wherein the treatment with the agonist or agent protects non-neoplastic cells from killing by the one or more anti-cancer therapeutics.
80. The method of any preceding paragraphs, wherein the cell, tissue or organ is of human origin.
81. The method of any preceding paragraphs, wherein the cell, tissue or organ is of non-human origin.
82. A composition comprising a live explanted cell, tissue or organ in contact with a δ-opioid receptor agonist, wherein the agonist is present in an amount sufficient to alter the function of at least one ion channel selected from the group consisting of EAAT1 ion channel and NCX1 ion channel; or
   an agent that alters the function of at least one ion channel selected from the group consisting of EAAT1 ion channel and NCX1 ion channel
83. The method of any preceding paragraphs, wherein the composition does not further comprise local anesthetic, an anti-arrhythmic, citrate, or magnesium.
84. The method of any preceding paragraphs, further comprising at least a second agent that alters the function of at least one ion channel selected from the group consisting of EAAT1 ion channel and NCX1 ion channel
85. The method of any preceding paragraphs, further comprising deuterium oxide.
86. The method of any preceding paragraphs, wherein the cell, tissue or organ is of human origin.
87. The method of any preceding paragraphs, wherein the cell, tissue or organ is of non-human origin.
88. A method of preserving viability or function of a cell, tissue or organ, the method comprising contacting the cell, tissue or organ in need of such preservation with an agonist of the δ-opioid receptor.
89. A method of cell, tissue or organ transplant, the method comprising contacting a donor cell, tissue or organ in situ or ex vivo with an agonist of the δ-opioid receptor.
90. A method of preparing a cell, tissue or organ for transplant, the method comprising contacting a donor cell, tissue or organ with an agonist of the δ-opioid receptor.
91. The method of any preceding paragraphs, wherein the tissue is an endoderm tissue, a mesoderm tissue, or an ectoderm tissue.
92. The method of any preceding paragraphs, wherein the tissue is selected from the group consisting of cornea, bone, cartilage, tendon, pancreas islet, heart valve, nerve, vascular, deep tissue flap, fat tissue, muscle, and vein.
93. The method of any preceding paragraphs, wherein the organ is selected from the group consisting of intestine, stomach, heart, kidney, bladder, pancreas, liver, lung, brain, skin, uterus, digit, and limb.
94. The method of any preceding paragraphs, wherein the contacting suppresses the metabolism or induces biostasis of the cell, tissue or organ.
95. The method of any preceding paragraphs, wherein the agonist is SNC-80.
96. The method of any preceding paragraphs, wherein the agonist is a derivative, analog, or variant of SNC-80.
97. The method of any preceding paragraphs, wherein the agonist is a derivative, analog, or variant of SNC-80 that binds and activates signaling by the δ-opioid receptor.
98. The method of any preceding paragraphs, wherein the agonist is administered at 100 μM.
99. A method of inducing biostasis in a cell, tissue or organ, the method comprising contacting the cell, tissue or organ in need of preservation with SNC-80 or donepezil, wherein the contacted cell, tissue or organ exhibits biostasis.
100. A method of preserving viability or function of a cell, tissue or organ, the method comprising contacting the cell, tissue or organ in need of such preservation with SNC-80 or donepezil.
101. A method of cell, tissue or organ transplant, the method comprising contacting a donor cell, tissue or organ in situ or ex vivo with SNC-80 or donepezil.
102. A method of preparing a cell, tissue or organ for transplant, the method comprising contacting a donor cell, tissue or organ with SNC-80 or donepezil.
103. A method of preserving healthy cells in a subject undergoing a cancer treatment, the method comprising administering to the subject receiving or to receive an anti-cancer therapy SNC-80 or donepezil.
104. A method of treating a hematological neoplastic disease, the method comprising harvesting bone marrow from a subject having such a disease, contacting harvested bone marrow or a cellular fraction thereof with SNC-80 or donepezil and with one or more anti-cancer therapeutics at a dose sufficient to kill neoplastic cells, treating the subject with chemotherapy or radiation sufficient to kill remaining bone marrow hematologic stem cells, and then administering the contacted bone marrow or cellular fraction to the subject.
105. A composition comprising a live explanted cell, tissue or organ in contact with SNC-80 or donepezil.
106. A method of cell, tissue or organ transplant, the method comprising contacting a donor cell, tissue or organ in situ or ex vivo with SNC-80 or donepezil.
107. A method of preparing a cell, tissue or organ for transplant, the method comprising contacting a donor cell, tissue or organ with SNC-80 or donepezil. 108.
109. A method of slowing a viral infection in a subject, the method comprising administering SNC-80 to a subject in need thereof.
110. A method of slowing a viral infection in a subject, the method comprising administering Donepezil to a subject in need thereof
111. The method of any preceding paragraphs, wherein the subject has or is at risk of having a viral infection.
112. The method of any preceding paragraphs, wherein the administration is local or systemic.
113. A method of slowing a viral infection in an organ or tissue, the method comprising contacting the organ or tissue with SNC-80.
114. A method of slowing a viral infection in an organ or tissue, the method comprising contacting the organ or tissue with Donepezil.
115. A method of recovering a cell, tissue or organ that has been contacted by an agonist of the δ-opioid receptor, wherein the agonist is administered at a dose sufficient to alter the function of at least one ion channel selected from the group consisting of EAAT1 ion channel and NCX1 ion channel or an agent that alters the function of at least one ion channel selected from the group consisting of EAAT1 ion channel and NCX1 ion channel, the method comprising contacting the cell, tissue or organ with a polyol.
116. A method of restoring normal metabolic function in a cell, tissue or organ that has been contacted by an agonist of the δ-opioid receptor, wherein the agonist is administered at a dose sufficient to alter the function of at least one ion channel selected from the group consisting of EAAT1 ion channel and NCX1 ion channel or an agent that alters the function of at least one ion channel selected from the group consisting of EAAT1 ion channel and NCX1 ion channel, the method comprising contacting the cell, tissue or organ with a polyol.
117. A method of restoring oxidative metabolism in a cell, tissue or organ that has been contacted by an agonist of the δ-opioid receptor, wherein the agonist is administered at a dose sufficient to alter the function of at least one ion channel selected from the group consisting of EAAT1 ion channel and NCX1 ion channel or an agent that alters the function of at least one ion channel selected from the group consisting of EAAT1 ion channel and NCX1 ion channel, the method comprising contacting the cell, tissue or organ with a polyol.
118. A method of restoring metabolic function is recovering a cell, tissue or organ that has been contacted by an agonist of the δ-opioid receptor to induce biostasis, wherein the agonist is administered at a dose sufficient to alter the function of at least one ion channel selected from the group consisting of EAAT1 ion channel and NCX1 ion channel or an agent that alters the function of at least one ion channel selected from the group consisting of EAAT1 ion channel and NCX1 ion channel, the method comprising contacting the cell, tissue or organ with a polyol.
119. The method of any preceding paragraphs, further comprising the step, prior to contact with the polyol, of removing the agonist or agent from the organ or tissue.
120. The method of any preceding paragraphs, wherein the polyol is kestose or erlose.

### EXAMPLES

### Example 1

Nature-inspired investigations into inducing a torpor-like state have relied on bear, ground squirrel, lemur, and other animal physiological changes to identify molecular mediators and triggers of a biostasis-like state; these triggers include hypothermia, hydrogen sulfide and carbon monoxide gases, AMP, hormones and neural signaling molecules, including delta-opioid, thyroid hormones and derivatives, and other interventions (e.g., Andrews, M. T. (2007) “Advances in molecular biology of hibernation in mammals” BioEssays, Review Article, which is incorporated herein by reference). Importantly, several interventions have been shown to rapidly induce synthetic torpor-like states in mice and other mammals, and this form of biostasis can help animals withstand lethal doses of radiation (REF). Interventions derived from analysis of torpor inducers also have been shown to significantly improve hepatocyte (liver epithelial cell) viability for cryopreservation, rat limb viability for cold storage over days, and protect against ischemic reperfusion injury following stroke. In addition, researchers have carried out transcriptomic analyses to define how genome-wide gene expression profiles change during induction of biostasis in some of these animal models (e.g., Arctic ground squirrels), which have identified key genes involved in redox cycling and glucose utilization, as well as transcriptomic signatures related to sleep deprivation, cold exposure, and calorie restriction. The PPAR-γ receptor also plays an important role in regulation of the metabolic state in hibernating animals, coupled with increased lipid metabolism that has been associated with decreased risk of ischemic reperfusion injury.

δ-opioids have been investigated due to the finding of natural δ-opioid modulation in hibernation and torpor, and the dosing of animals with δ-opioid agonists, such as DADLE and other peptides, has been shown to reduce core body temperature (Rawls, S. M., Hewson, J. M., Ivan, S. and Cowan, A. (2005) “Brain delta2 opioid receptors mediate SNC-80-evoked hypothermia in rats” Brain Research, July 5 (1049) 61:69, which is incorporated herein by reference). This work was performed to investigate the observations of δ-opioid agonists generally inducing hypothermia immediately following dosing. The authors found that indeed delta2 opioid receptors in the brain were responsible for the hypothermia response. Chemical agonists specific to δ-opioid receptors also have been developed for non-addicting pain treatment, including the compound SNC-80 (Bilsky, E J, et al. Journal of Pharmacology and Experimental Therapeutics. 1995. 273(1) 359-366. While this compound also has been noted to induce hypothermia in rats via the delta-2 opioid receptor (Rawls, S. M., Hewson, J. M., Iran, S. and Cowan, A. (2005) “Brain delta2 opioid receptors mediate SNC-80-evoked hypothermia in rats” Brain Research, July 5 (1049) 61:69, which is incorporated herein by reference), this molecule has never been explored or suggested to be useful to induce a hypometabolic state for tissue, organ, limb, or whole organism preservation.

Demonstrated herein is the ability of SNC-80 to induce a torpor-like hypometabolic state in cell culture and whole animal (tadpole) models. It was shown herein that the compound results in a dose-dependent reduction in oxygen consumption and other metrics of metabolic rate in health tissues and whole organism (Xenopus tadpoles). Importantly, the compound at higher concentrations (>100 uM) results in arrest of tadpole movement that can be subsequently reversed by removal of the drug. It was also shown that the dosing of SNC-80 on Xenopus embryos results in an approximately 50% similarity of the resulting gene regulatory network compared to Arctic ground squirrels during torpor, indicating that the drug indeed mimics torpor mechanistically to induce a similar physiological profile. Interestingly, while it was found that SNC-80 slows metabolism of cultured intestinal cancer cells (Caco-2 cells) as measured by a reduction in intracellular ATP levels, it did not inhibit oxygen utilization in these transformed cells. Unlike previous studies (e.g., Andrews, M. T. (2007) “Advances in molecular biology of hibernation in mammals” BioEssays, Review Article, which is incorporated herein by reference), this indicates that a somatic, non-brain mechanism could be responsible for the hypometabolic state that was observed, in contrast to the demonstration that central nervous system involvement is critical.

It is specifically contemplated herein that the biostasis compound described herein can be used to protect normal cells from anti-cancer therapies, such as radiation and chemotherapies that induce oxygen free radical generation, and thereby increasing their therapeutic efficacy. The SNC-80 compound suppresses metabolism via internal molecular mechanisms in a stable, reversible manner for stabilization of cells, tissues, organs, tissues, and whole organisms, as well as for the effectiveness of anti-cancer therapies by protecting normal cells against injury.

### Example 2

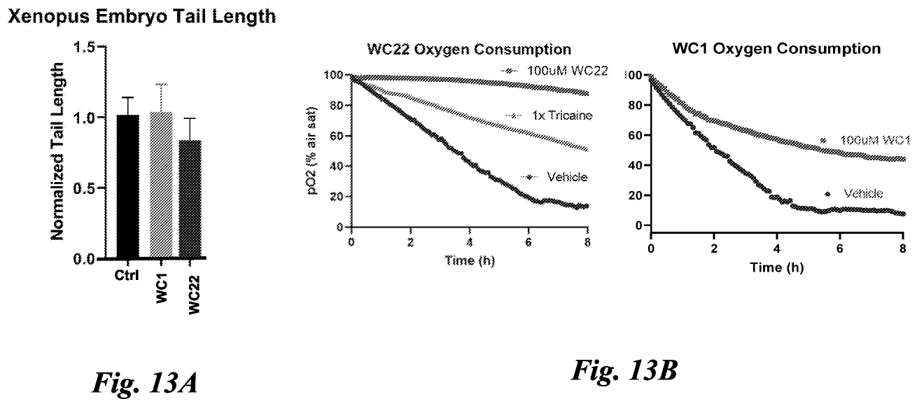

Donepezil is identified herein as an agent that can induce biostasis. To confirm whether Donepezil functions in a manner similar to SNC-80 (e.g., to induce biostasis), its effect was tested on Xenopus tadpoles. The tadpoles were treated with Donepezil to determine if the drug would slow their development, similar to what was observed with SNC-80. Donepezil-treated tadpoles had reduced tail length as compared to a vehicle control or SNC-80 over a given time period, indicating that Donepezil slows the growth rate of Xenopus tadpoles (FIG. 13A).

Next, tadpoles were treated with Donepezil to determine if the drug would slow consumption of oxygen. The oxygen consumption assay described herein above was used. Treatment with Donepezil markedly reduced oxygen consumption in the tadpoles as compared to a vehicle control (FIG. 13B). To confirm that this effect observed with the drug is not due to lack of motion alone, Xenopus tadpoles were treated with the anesthetic, Tricaine. Donepezil treatment reduced oxygen consumption in Xenopus tadpoles greater than treatment with 1×Tricaine, confirming that the observed effect is not due to lack of motion (FIG. 13B).

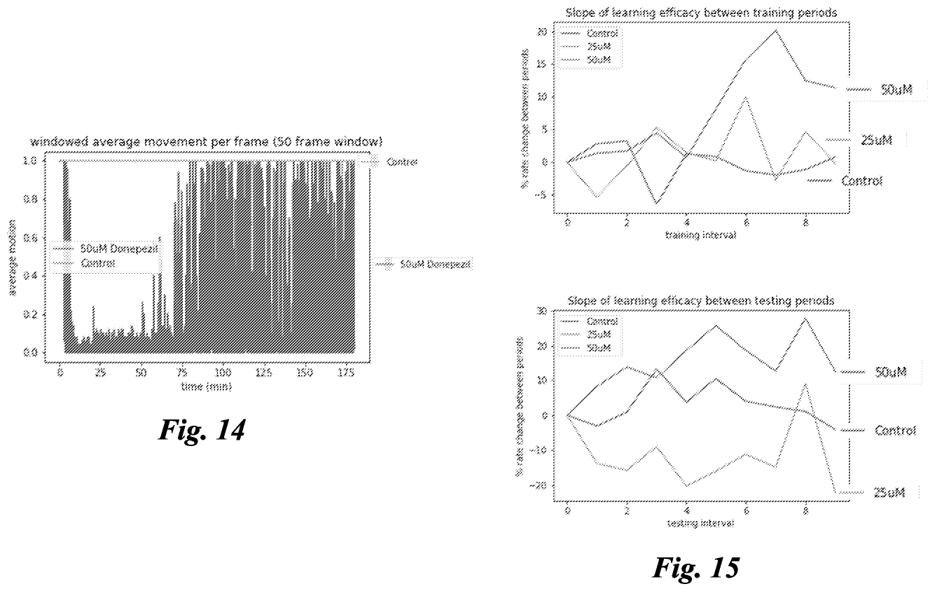

Tadpoles treated with Donepezil exhibited a dramatic slowing of movement; slowing occurred within 10 min following 50 uM Donepezil (FIG. 14). To determine if this slowing of movement is reversible, the drug was removed from the culture medium after 30 minutes (denoted by the hashed-line). Movement resumed at the pre-treatment level within 50 min after drug removal. These data show that the slowing of movement caused by Donepezil is reversible.

Finally, the cognitive and motor function performance of Xenopus tadpoles treated with Donepezil was assessed. Tadpoles were exposed to 25 uM or 50 uM Donepezil for 30 min to induce stasis. Following the onset of stasis, the drug was removed and tadpoles were allowed to recover. Tadpoles were then tested over a 24 hour period in an automated behavior and cognitive testing platform to measure whether tadpoles that previously went through stasis had decreased cognitive and motor function performance. The data presented in FIG. 15 show that Donepezil-treated tadpoles did not have decreased cognitive or motor function performance as compared to the untreated control tadpoles. In fact, the data indicate that 50 uM Donepezil treatment may have slightly improved cognitive performance.

### Example 3

Protocol for Evaluating the Effect of an Agent on Inducing Biostasis of a Pig Limb.

The establishment of biostasis in a mammal according to the methods described herein was evaluated in a pig limb model. Prior to removing a hind limb from the animal, a pre-operation blood draw was preformed to obtain a baseline. The hind limb was removed via an amputation, and four muscle biopsies were immediately taken. For use in RT-PCR, a first biopsy was stored at −80° C. to preserve RNA integrity. For histology, a second biopsy was fixed in formalin and stored at 2-4° C. or at room temperature. For use in ELISA, a third biopsy was snap frozen and stored at −80° C. Finally, in lieu of Microdialysis, a fourth biopsy was snap frozen and stored at −80° C.

The limb was first flushed with 4° C. heparinized Krebs Henseleit Buffer alone. The limb was then weighed and attached to the ULiSSES Platform. The Platform was run subnormothermic relative to room temp (e.g., 18-23° C.). During the protocol, the limb was perfused with buffer alone (control) or buffer with SNC-80. Further description of the ULiSSES Platform can be found on the world wide web at www.techbriefs.com/component/content/article/tb/stories/blog/35406.

Following the limb being attached to the platform (time 0 hour), an RNA-preserving biopsy, a formalin-fixed biopsy, and a snap frozen biopsy were taken, and a perfusate sample was also taken. The “perfusate sample” is a sample that is assessed for arterial and vascular blood gases, and subjected to a blood chemistry panel to measure sodium, potassium, glucose, and lactate levels. The perfusate sample is further snap frozen for other analyses, including RNAseq and cytokine analysis.

These sampling steps were further repeated at times 3 hours, 6 hours, 12 hours, and 24 hours. At times 9 hours, 15 hours, 18 hours, and 21 hours, a perfusate sample was taken.

The protocol was completed at time 24 hours. The limb was removed from the ULiSSES Platform and weighed. Separate, proof of concept experiments were performed as described above, but were completed at time 6 hours.

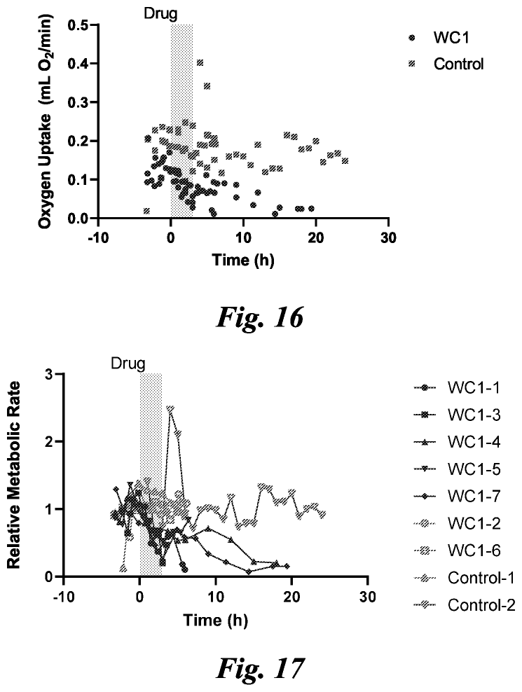

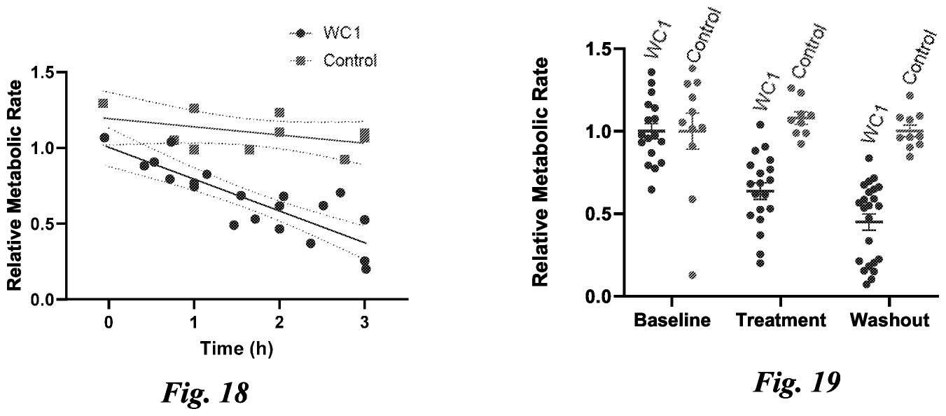

Following the additional of SNC-80, oxygen uptake levels begin to decline, with a noticeable reduction at 10 hours post-treatment, and a marked reduction at 24 hours post-treatment in the limb, as compared to a control treated limb (e.g., a deactivated form of SNC-80 that has no effect) (FIG. 16). In fact, SNC-80 was capable of reducing oxygen uptake levels to a greater extent than hypothermic conditions, which is a known standard in the art for inducing biostasis (data not shown). The metabolic rate of the limb was also reduced in the SNC-80-treated limb as compared to a control-treated limb, with a noticeable reduction occurring at 3 hours post-treatment (FIGS. 17-19). Together, these data indicated that SNC-80 had induced biostasis in the limb.

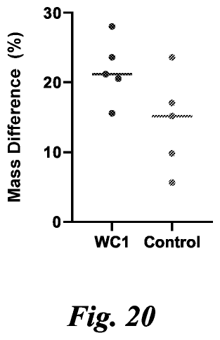

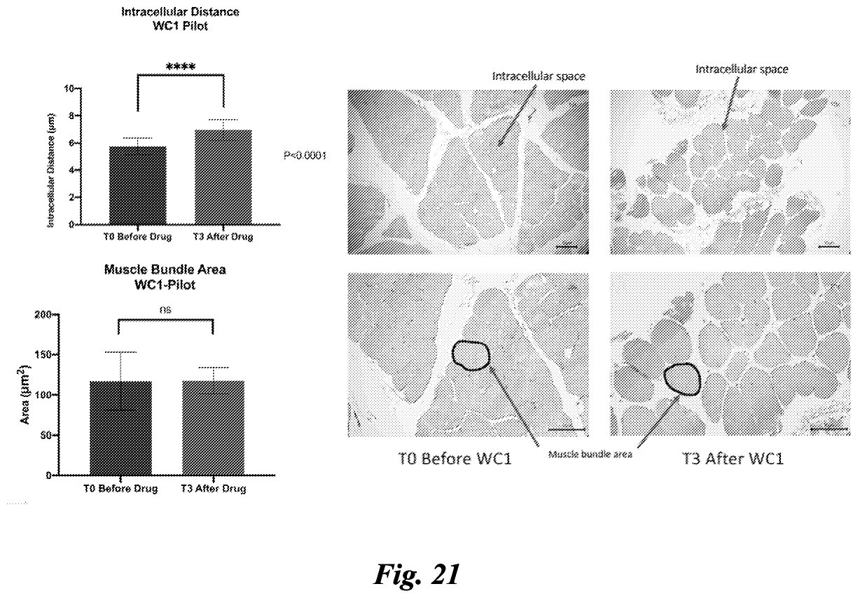

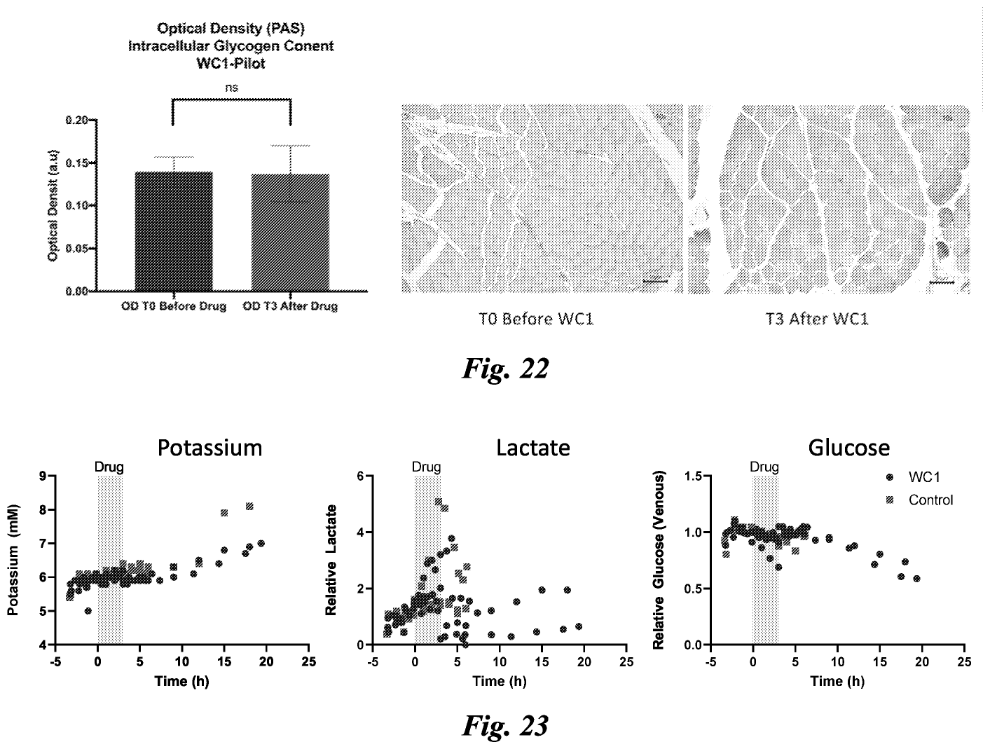

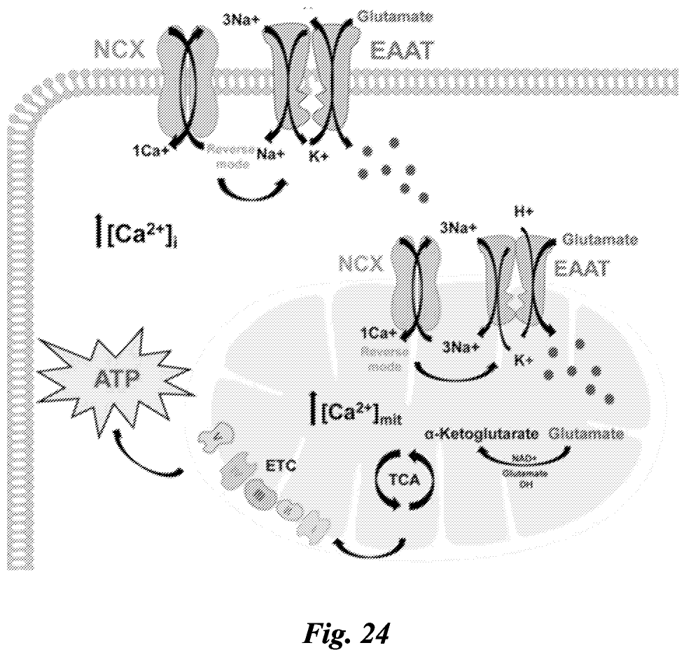

When comparing the mass difference of the limb at the start and end of the experiment, a greater difference was observed following treatment with SNC-80 as compared to the control-treated limb, however, it was noted that limb edema was observed in the fascia of the treated limb and not in the muscle (FIG. 20). There was an observed increase in intercellular edema that corresponds to weight increase observed in limbs (FIG. 21). Muscle bundle area did not increase significantly following treatment with SNC-90, indicating that the muscle remains intact following induction of biostasis (FIG. 22). Further, there was not a marked difference in the levels of potassium, lactate, or glucose following treatment with SNC-80 as compared to the control-treated limb (FIG. 23). Together, these indicate that the limb was healthy during induced biostasis and that the reduction in oxygen uptake and metabolic rate was not due to death of the tissue.

### Example 4

Synergy Between SNC-80 and KB-R7943 Mesylate

The effects of SNC-80 and the NCX1 inhibitor KB-R7943 mesylate (also referred to herein as “WC-60) were evaluated alone and in combination. Tadpoles were exposed to SNC-80 at 25 μM for 24 hr continuously, and swimming was assessed. SNC-80 at 25 μM is not sufficient to induce biostasis, and the tadpoles exhibit normal swimming and oxygen consumption (FIG. 25A). Similarly, no effect on oxygen consumption was observed when tadpoles were exposed to KB-R7943 mesylate at 35 uM for 24 h continuously (FIG. 25B). Tadpoles contacted with KB-R7943 mesylate, show decreased swimming (data not shown).

Strikingly, when exposed to combination treatment with 25 μM SNC-80 and 35 μM KB-R7943 mesylate, tadpoles show decreased oxygen consumption (FIG. 25C) and reduction in motion (data not shown), indicating an additive, or synergistic effect.

### Example 5

Polyols Accelerate Biostasis Recovery in Tadpoles

To determine if an agent plays a role in accelerating recovery following induction of biostasis, tadpoles can be treated with a biostasis-inducing agent as described herein, to induce biostasis and then transferred to a medium containing a test agent, or just medium. Recovery is assessed by assessing development rate following recovery (e.g., by measuring tadpole tail length), movement, and oxygen consumption.

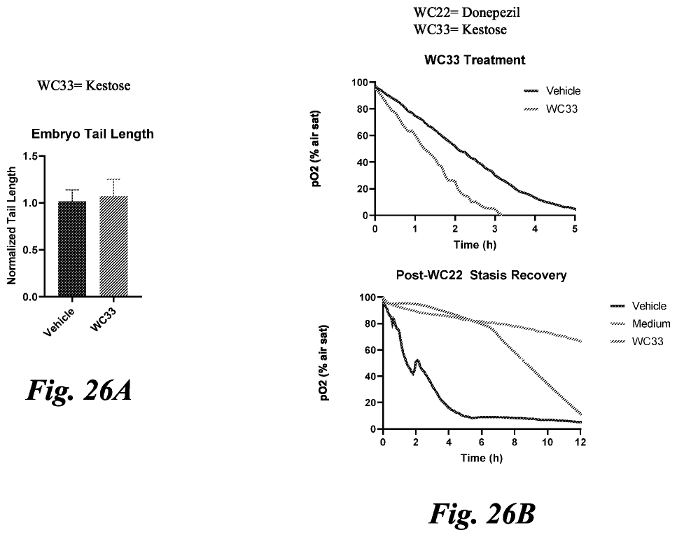

Tadpoles treated with Donepezil to induce biostasis were transferred to Marc's Modified Ringer's (MMR) medium (0.1 M NaCl, 2.0 mM KCl, 1 mM MgSO4, 2 mM CaCl2, 5 mM HEPES (pH 7.8); adjusted to pH 7.4) alone or MMR containing the polyol kestose at 50 mM. Embryos exposed to Kestose for 24 h showed an increased development via an increased tail length as compared to MMR alone (FIG. 26A). Tadpoles in Donepezil-induced biostasis that were then contacted with MMR plus 50 mM kestose showed an increased rate of oxygen consumption as compared to MMR alone (FIG. 26B). These data indicated that tadpoles treated with kestose during recovery from biostasis induced via Donepezil show faster recovery as compared to tadpoles recovered in MMR alone.

### Example 6

Deuterium Oxide (²H₂O) Slows Development in Xenopus Embryos

Deuterium oxide, ²H₂O, is commonly known as heavy water. Deuterium oxide has previously been shown to alter biological time by disrupting circadian & cell cycles.

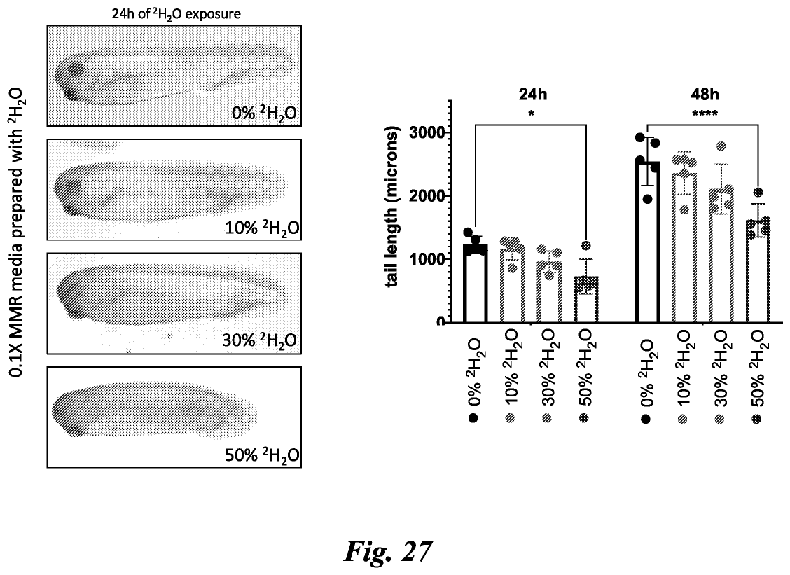

The effects of ²H₂O on metabolism were examined in relation to biostasis in vivo. Xenopus embryos were contacted with 0.1×MMR media prepared with various concentrations of ²H₂O. A dosage-dependent effect was observed when assessing embryo length, a standard assay to assess embryo development. 50% ²H₂O resulted in a marked reduction in embryo length, indicating that this dose slowed development of the embryo (FIG. 27). Concentrations<50% ²H₂O had no effect on tail length, while concentrations>70% ²H₂O were toxic (data not shown).

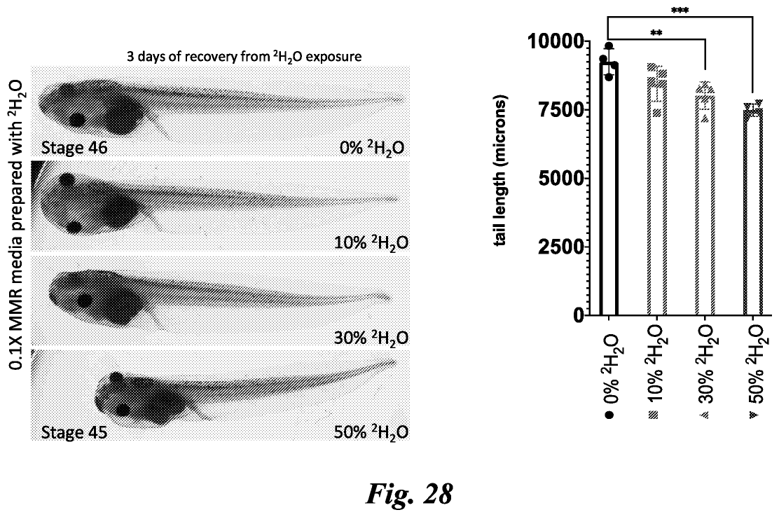

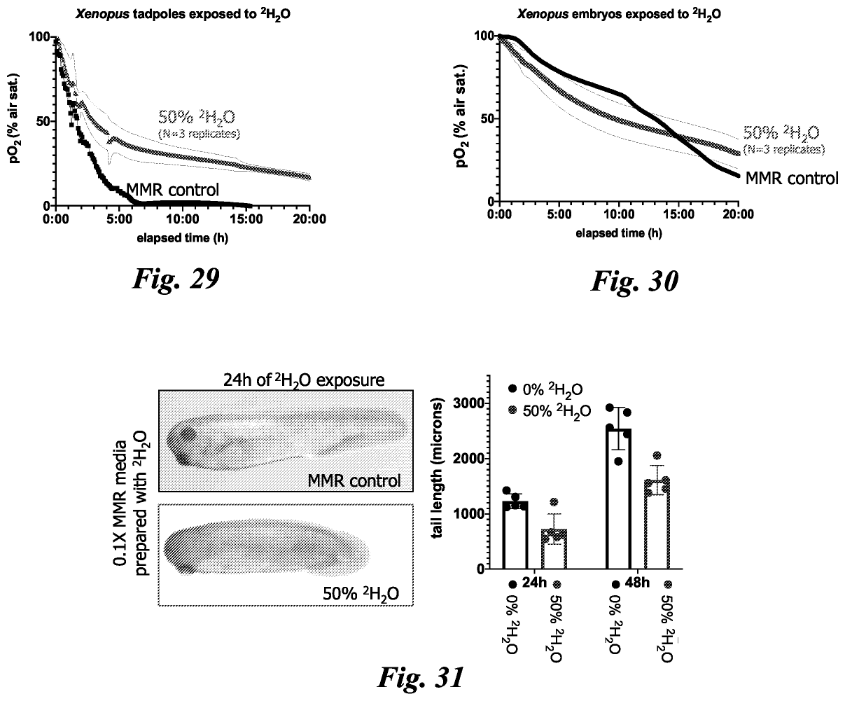

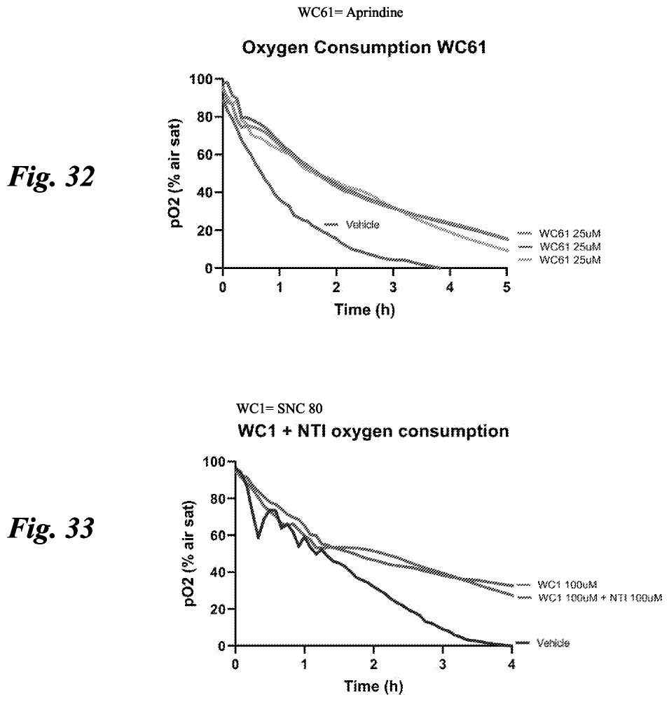

Importantly, no adverse effects are observed following recovery. Embryos that were previously in biostasis for four days were allowed to recover for three days following ²H₂O exposure. Embryos contacted with 50% ²H₂O remain developmentally delayed after 3 days of recovery but do not exhibit adverse effects (FIG. 28). These data indicated that the reduced tail length observed in FIG. 27 is not due to a developmental defect, but rather a delay in development.

When tadpoles were exposed to 50% ²H₂O, swimming was slowed within 10 minutes. When tadpoles were returned to normal media (i.e., without ²H₂O), swimming returned to normal (data not shown). Concentrations<50% ²H₂O had no effect on swimming, while concentrations>70% ²H₂O were toxic.

To determine if there is a synergistic effect between Donepezil and ²H₂O, Xenopus embryos are contacted with 0.1×MMR media prepared with 50% ²H₂O and either 40 μM, 20 μM, or 10 μM Donepazil, and 25% ²H₂O and either 40 μM, 20 μM, or 10 μM Donepezil. Oxygen consumption is measured to determine if metabolism is slowing in the embryos following contact; a reduction in oxygen consumption indicates slowed metabolism. A dosage-dependent effect is observed when assessing embryo length, a standard assay to assess embryo development, and oxygen consumption. A marked reduction in embryo length indicates that the indicated dose slowed development of the embryo.

TABLE 1

| genes | probability | log2fc | pvalue | statistic |
|---|---|---|---|---|
| DNAJA1 | 0.990828 | 20796.27 | 0.009172 | −3.5643 |
| CTH | 0.9654 | 8255.849 | 0.0346 | −2.61653 |
| MMS19 | 0.954694 | 7257.633 | 0.045306 | −2.53193 |
| HNRNPH2 | 0.976672 | 5443.447 | 0.023328 | −2.98624 |
| SFXN4 | 0.969207 | 5099.76 | 0.030793 | −2.96051 |
| OR1E3 | 0.991643 | 4271.6 | 0.008357 | −3.70528 |
| AQP5 | 0.994708 | 4206.323 | 0.005292 | −4.82664 |
| PPIC | 0.990589 | 3717.504 | 0.009411 | −3.56378 |
| TNF | 0.968859 | 3666.533 | 0.031141 | −2.80502 |
| MDH2 | 0.981377 | 3484.991 | 0.018623 | −3.19414 |
| AMOT | 0.989275 | 3344.572 | 0.010725 | −3.82254 |
| CHRND | 0.999599 | 3077.41 | 0.000401 | −6.40391 |
| NREP | 0.97992 | 2868.33 | 0.02008 | −3.02352 |
| PRKCSH | 0.997738 | 2832.411 | 0.002262 | −5.45949 |
| USP26 | 0.997322 | 2785.244 | 0.002678 | −4.60431 |
| ZBTB25 | 0.972327 | 2766.12 | 0.027673 | −2.99176 |
| RTL1 | 0.952991 | 2595.789 | 0.047009 | −2.42026 |
| COL2A1 | 0.951911 | 2512.746 | 0.048089 | −2.44918 |
| MUC19 | 0.979471 | 2334.421 | 0.020529 | −3.01619 |
| ICAM1 | 0.972108 | 2255.307 | 0.027892 | −2.78818 |
| CGB7 | 0.958796 | 2249.556 | 0.041204 | −3.31118 |
| EMSY | 0.971144 | 2221.006 | 0.028856 | −2.94756 |
| BNIP1 | 0.963278 | 2128.223 | 0.036722 | −3.47677 |
| VGF | 0.993892 | 2005.863 | 0.006108 | −3.87296 |
| KIF20B | 0.994506 | 1853.966 | 0.005494 | −3.97579 |
| BAZ1A | 0.988289 | 1831.775 | 0.011711 | −3.57763 |
| TES | 0.99031 | 1742.818 | 0.00969 | −3.52296 |
| OTUD7B | 0.984437 | 1651.693 | 0.015563 | −3.27163 |
| JMJD8 | 0.977238 | 1614.136 | 0.022762 | −3.28719 |
| FER1L5 | 0.996175 | 1559.499 | 0.003825 | −4.24669 |
| CLIC4 | 0.96999 | 1482.593 | 0.03001 | −2.74718 |
| CKB | 0.989218 | 1459.985 | 0.010782 | −3.70248 |
| KRTAP10-1 | 0.974154 | 1441.004 | 0.025846 | −2.84137 |
| CHSY1 | 0.98233 | 1417.852 | 0.01767 | −3.46184 |
| RTN1 | 0.979482 | 1395.177 | 0.020518 | −3.22852 |
| HAX1 | 0.95028 | 1356.836 | 0.04972 | −2.40325 |
| GBE1 | 0.975167 | 1296.876 | 0.024833 | −3.54645 |
| IGF1R | 0.977861 | 1292.103 | 0.022139 | −3.52089 |
| ACTN4 | 0.955639 | 1251.416 | 0.044361 | −4.55613 |
| PCDHGB1 | 0.962486 | 1163.875 | 0.037514 | −2.69164 |
| BARX1 | 0.977269 | 1143.729 | 0.022731 | −3.23584 |
| STAU2 | 0.974525 | 1120.613 | 0.025475 | −2.91752 |
| PLS1 | 0.950043 | 1101.556 | 0.049957 | −2.37212 |
| CCNC | 0.959549 | 1099.779 | 0.040451 | −2.51 |
| CDH5 | 0.959998 | 1072.8 | 0.040002 | −2.64271 |
| ADH4 | 0.986492 | 1054.74 | 0.013508 | −3.39091 |
| ARHGAP11B | 0.979046 | 1048.186 | 0.020954 | −3.14444 |
| TXNRD2 | 0.962388 | 1037.289 | 0.037612 | −4.09482 |
| CDR1 | 0.961974 | 1032.989 | 0.038026 | −2.61754 |
| HIST1H2BF | 0.991337 | 1024.575 | 0.008663 | −3.61462 |
| KIF14 | 0.992651 | 1008.855 | 0.007349 | −3.87728 |
| SDCBP | 0.95473 | 963.628 | 0.04527 | −2.52489 |
| CTNNB1 | 0.980978 | 963.2702 | 0.019022 | −3.37804 |
| EIF3F | 0.95729 | 941.1783 | 0.04271 | −2.47213 |
| SIGMAR1 | 0.991221 | 929.9472 | 0.008779 | −3.63726 |
| KAT6B | 0.971883 | 908.5141 | 0.028117 | −2.75953 |
| LATS1 | 0.952587 | 905.685 | 0.047413 | −2.49015 |
| EIF1AY | 0.989871 | 899.3523 | 0.010129 | −3.55456 |
| VAV1 | 0.968482 | 894.553 | 0.031518 | −2.74578 |
| LRR1 | 0.999819 | 887.897 | 0.000181 | −8.89232 |
| DDX21 | 0.967305 | 868.8978 | 0.032695 | −2.67125 |
| ZBED1 | 0.996421 | 866.9522 | 0.003579 | −4.38906 |
| C20orf96 | 0.984301 | 860.0427 | 0.015699 | −3.2521 |
| MAZ | 0.952506 | 857.3719 | 0.047494 | −2.56964 |
| ZFR | 0.972526 | 853.9934 | 0.027474 | −2.8456 |
| RPL19 | 0.982105 | 841.3602 | 0.017895 | −3.23988 |
| IL1RAPL2 | 0.984716 | 806.4431 | 0.015284 | −3.25083 |
| ABI1 | 0.98829 | 801.8464 | 0.01171 | −3.67733 |
| ARHGAP17 | 0.995759 | 788.4573 | 0.004241 | −4.18474 |
| ZNF101 | 0.982346 | 787.7146 | 0.017654 | −3.1151 |
| TEX13B | 0.974816 | 786.2123 | 0.025184 | −2.92443 |
| DNASE1 | 0.981395 | 782.9358 | 0.018605 | −3.26977 |
| OR5C1 | 0.96112 | 776.4814 | 0.03888 | −2.72415 |
| IKZF2 | 0.976161 | 772.1323 | 0.023839 | −2.88285 |
| HADH | 0.985683 | 770.7671 | 0.014317 | −3.30277 |
| CD63 | 0.995105 | 765.8309 | 0.004895 | −4.19311 |
| WNT7B | 0.984064 | 750.7568 | 0.015936 | −3.16085 |
| DNMT3A | 0.963967 | 748.5222 | 0.036033 | −2.82491 |
| CHRNA9 | 0.993406 | 739.5632 | 0.006594 | −3.96748 |
| GJA5 | 0.997529 | 730.9074 | 0.002471 | −5.47024 |
| PRND | 0.967416 | 729.9308 | 0.032584 | −3.76625 |
| RNASE8 | 0.981145 | 724.8077 | 0.018855 | −3.19602 |
| DEFB125 | 0.991818 | 724.6557 | 0.008182 | −5.4168 |
| MYORG | 0.976139 | 700.1136 | 0.023861 | −2.87383 |
| GPR88 | 0.959598 | 700.0657 | 0.040402 | −3.03838 |
| CD99L2 | 0.967449 | 676.5137 | 0.032551 | −2.65859 |
| SP6 | 0.974425 | 666.6328 | 0.025575 | −2.85182 |
| FLG | 0.986604 | 665.8964 | 0.013396 | −3.3703 |
| CCNB2 | 0.982625 | 663.6626 | 0.017375 | −3.12278 |
| CETN3 | 0.957683 | 663.1214 | 0.042317 | −2.53792 |
| SMURF2 | 0.977265 | 660.967 | 0.022735 | −3.09311 |
| SLC1A4 | 0.969949 | 648.492 | 0.030051 | −2.71355 |
| FBLN1 | 0.955519 | 629.5582 | 0.044481 | −2.61324 |
| GAR1 | 0.969915 | 623.2591 | 0.030085 | −2.92858 |
| NTM | 0.953802 | 618.5448 | 0.046198 | −2.51839 |
| VEGFC | 0.967537 | 618.0334 | 0.032463 | −2.7171 |
| ADGRA2 | 0.976234 | 616.446 | 0.023766 | −3.45043 |
| SPTSSA | 0.973512 | 609.0651 | 0.026488 | −3.04659 |
| ASB4 | 0.950282 | 603.8886 | 0.049718 | −2.53087 |
| VPS45 | 0.997181 | 593.4094 | 0.002819 | −4.53589 |
| AZGP1 | 0.991631 | 591.2723 | 0.008369 | −3.7801 |
| HIST1H2BO | 0.998861 | 588.9995 | 0.001139 | −5.49824 |
| KMT2A | 0.974958 | 587.3251 | 0.025042 | −2.92512 |
| PFKP | 0.996354 | 587.314 | 0.003646 | −4.40684 |
| METTL3 | 0.997235 | 571.9354 | 0.002765 | −4.53234 |
| MTNR1A | 0.994606 | 566.8203 | 0.005394 | −4.5303 |
| IGSF3 | 0.984375 | 560.0145 | 0.015625 | −4.41884 |
| SNRPF | 0.996601 | 546.4338 | 0.003399 | −4.43156 |
| ZNF330 | 0.984295 | 541.4492 | 0.015705 | −3.27736 |
| BPIFB3 | 0.996794 | 531.5301 | 0.003206 | −5.00681 |
| OR1L6 | 0.999632 | 531.0264 | 0.000368 | −6.48144 |
| ZNF107 | 0.97386 | 529.8146 | 0.02614 | −2.81035 |
| IL5RA | 0.994651 | 529.1886 | 0.005349 | −4.02692 |
| PLCD1 | 0.999833 | 528.9783 | 0.000167 | −7.28233 |
| ACTN2 | 0.995399 | 528.7876 | 0.004601 | −4.60171 |
| UNC13B | 0.994901 | 525.0144 | 0.005099 | −4.67767 |
| MAGEB17 | 0.983427 | 515.1281 | 0.016573 | −3.17226 |
| CAPZA1 | 0.965473 | 514.3723 | 0.034527 | −2.63156 |
| ADCY4 | 0.999932 | 506.5547 | 6.84E−05 | −8.47549 |
| ARC | 0.969525 | 504.9127 | 0.030475 | −2.91998 |
| RNASE2 | 0.952627 | 495.1782 | 0.047373 | −2.53458 |
| CADM1 | 0.960814 | 484.9494 | 0.039186 | −2.54393 |
| PLK4 | 0.952322 | 483.7561 | 0.047678 | −2.45705 |
| DSP | 0.960944 | 475.4615 | 0.039056 | −2.78625 |
| POGLUT2 | 0.958603 | 473.5233 | 0.041397 | −2.55568 |
| CDC5L | 0.962842 | 471.6536 | 0.037158 | −2.61653 |
| RALGAPA1 | 0.96545 | 465.8702 | 0.03455 | −2.68374 |
| SFSWAP | 0.994134 | 460.7499 | 0.005866 | −3.97155 |
| DNAH5 | 0.998968 | 454.9711 | 0.001032 | −5.60363 |
| TIMM44 | 0.95171 | 453.0853 | 0.04829 | −2.44324 |
| SLC9A4 | 0.992714 | 445.0709 | 0.007286 | −3.93078 |
| GRHL2 | 0.958242 | 444.3173 | 0.041758 | −3.42468 |
| RGS10 | 0.987463 | 438.3125 | 0.012537 | −3.66632 |
| SSBP4 | 0.987963 | 434.3816 | 0.012037 | −3.45962 |
| ZFP37 | 0.978375 | 434.2665 | 0.021625 | −3.06116 |
| RFPL3S | 0.957721 | 431.8477 | 0.042279 | −2.50031 |
| MT-ND2 | 0.99049 | 429.7436 | 0.00951 | −3.57214 |
| PDGFB | 0.987857 | 429.1195 | 0.012143 | −3.38125 |
| CCDC198 | 0.954642 | 424.4016 | 0.045358 | −2.81242 |
| SURF2 | 0.990483 | 422.8514 | 0.009517 | −4.08268 |
| TRPC1 | 0.969639 | 418.574 | 0.030361 | −2.86004 |
| PCBP2 | 0.97817 | 417.238 | 0.02183 | −3.0205 |
| TTC7B | 0.956045 | 416.2767 | 0.043955 | −2.45838 |
| OCM | 0.999027 | 411.0356 | 0.000973 | −5.5125 |
| BTN3A2 | 0.974017 | 407.9211 | 0.025983 | −2.86975 |
| OR14A16 | 0.992263 | 406.7666 | 0.007737 | −3.71844 |
| FAU | 0.993816 | 404.9277 | 0.006184 | −3.86446 |
| DGKD | 0.978038 | 402.3742 | 0.021962 | −2.96082 |
| TBC1D25 | 0.985797 | 402.3024 | 0.014203 | −3.24929 |
| GFAP | 0.969816 | 401.7407 | 0.030184 | −2.76904 |
| OR5K1 | 0.965892 | 399.461 | 0.034108 | −2.73351 |
| NUDT9 | 0.997648 | 394.3051 | 0.002352 | −5.60396 |
| NR4A1 | 0.990077 | 391.9021 | 0.009923 | −3.52003 |
| GRTP1 | 0.966938 | 385.7811 | 0.033062 | −2.75962 |
| MATN2 | 0.999768 | 382.9696 | 0.000232 | −7.51138 |
| HIST1H2AM | 0.971485 | 382.8485 | 0.028515 | −3.04774 |
| ATG14 | 0.999817 | 380.1176 | 0.000183 | −9.20922 |
| ADAM15 | 0.977244 | 376.9274 | 0.022756 | −3.11981 |
| ZBTB11 | 0.993712 | 364.5345 | 0.006288 | −4.10252 |
| STXBP6 | 0.969234 | 363.4385 | 0.030766 | −2.86891 |
| NOP9 | 0.972209 | 360.526 | 0.027791 | −2.8839 |
| CRYBB2 | 0.976992 | 360.4018 | 0.023008 | −2.9002 |
| USP17L2 | 0.975929 | 355.6111 | 0.024071 | −2.98126 |
| TUBG2 | 0.971032 | 354.33 | 0.028968 | −2.75955 |
| DHX37 | 0.975916 | 349.4411 | 0.024084 | −2.9344 |
| FKBP11 | 0.976178 | 348.9298 | 0.023822 | −2.9024 |
| FKBP10 | 0.978662 | 348.73 | 0.021338 | −3.2174 |
| ADAM32 | 0.965781 | 345.9787 | 0.034219 | −2.64413 |
| GBA2 | 0.992159 | 344.8344 | 0.007841 | −3.87062 |
| HPS4 | 0.984345 | 344.4274 | 0.015655 | −3.33883 |
| MYO9A | 0.998219 | 341.9077 | 0.001781 | −5.54782 |
| ARHGDIA | 0.965809 | 337.2551 | 0.034191 | −2.63969 |
| HMGN2 | 0.967678 | 336.5003 | 0.032322 | −2.82146 |
| IFT81 | 0.99067 | 334.1153 | 0.00933 | −3.6139 |
| SSX4B | 0.964238 | 331.0541 | 0.035762 | −2.80463 |
| NAT2 | 0.990197 | 324.4835 | 0.009803 | −3.94286 |
| LAMP3 | 0.995657 | 324.3461 | 0.004343 | −4.20162 |
| CLCN1 | 0.981083 | 321.7569 | 0.018917 | −3.04045 |
| NDUFB2 | 0.987922 | 319.7844 | 0.012078 | −3.38734 |
| ACAA1 | 0.996369 | 317.0826 | 0.003631 | −4.52627 |
| OR5D13 | 0.982322 | 316.596 | 0.017678 | −3.08554 |
| ADAM12 | 0.991727 | 316.278 | 0.008273 | −3.71788 |
| PSMC2 | 0.976742 | 315.8595 | 0.023258 | −5.06096 |
| TNNT1 | 0.974709 | 311.9784 | 0.025291 | −2.85202 |
| DNAH3 | 0.978748 | 311.9069 | 0.021252 | −2.96646 |
| KCNMB1 | 0.969615 | 309.5169 | 0.030385 | −3.30735 |
| MYCBPAP | 0.961833 | 300.3926 | 0.038167 | −2.61088 |
| MYH2 | 0.961034 | 299.9586 | 0.038966 | −2.76489 |
| SLC29A1 | 0.966584 | 296.5846 | 0.033416 | −2.64471 |
| VPS16 | 0.954885 | 295.8953 | 0.045115 | −2.71105 |
| LIPH | 0.974799 | 295.8799 | 0.025201 | −3.04407 |
| ACTB | 0.999336 | 295.321 | 0.000664 | −7.34582 |
| MSH5 | 0.957341 | 294.0858 | 0.042659 | −2.5702 |
| ACTL6B | 0.992776 | 286.5148 | 0.007224 | −3.81566 |
| GABRR2 | 0.997516 | 285.4241 | 0.002484 | −5.48811 |
| HEBP2 | 0.998678 | 282.2399 | 0.001322 | −5.42225 |
| TRAPPC2B | 0.955887 | 282.0286 | 0.044113 | −2.45004 |
| SIGLEC9 | 0.984286 | 279.8052 | 0.015714 | −3.8955 |
| CA8 | 0.993781 | 279.1576 | 0.006219 | −3.86176 |
| ABCB8 | 0.986257 | 278.2124 | 0.013743 | −3.59461 |
| INTS10 | 0.990371 | 278.0881 | 0.009629 | −3.734 |
| SYCP2 | 0.983664 | 278.0133 | 0.016336 | −3.20007 |
| ACSL4 | 0.994575 | 277.421 | 0.005425 | −4.65055 |
| SNAPC1 | 0.987511 | 275.9322 | 0.012489 | −3.46189 |
| RNASE3 | 0.998608 | 275.7824 | 0.001392 | −5.48266 |
| MARCKSL1 | 0.976747 | 273.825 | 0.023253 | −2.91888 |
| KIF1A | 0.99839 | 272.7332 | 0.00161 | −5.40983 |
| PDK4 | 0.968733 | 272.3437 | 0.031267 | −2.82031 |
| PLEKHB1 | 0.96672 | 264.3014 | 0.03328 | −2.66939 |
| USP32 | 0.980053 | 263.5973 | 0.019947 | −3.08412 |
| MATK | 0.964671 | 261.6154 | 0.035329 | −2.72981 |
| PDK1 | 0.950457 | 260.3809 | 0.049543 | −2.38329 |
| PLEK | 0.975506 | 258.4891 | 0.024494 | −2.85558 |
| APOF | 0.987578 | 258.484 | 0.012422 | −3.34216 |
| PER3 | 0.95747 | 257.251 | 0.04253 | −2.48987 |
| OR4F3 | 0.959452 | 256.5513 | 0.040548 | −2.73625 |
| SH3GLB2 | 0.995055 | 254.4523 | 0.004945 | −4.0809 |
| VPS4A | 0.981993 | 254.0078 | 0.018007 | −3.07627 |
| PCDH9 | 0.998488 | 252.3576 | 0.001512 | −5.10338 |
| HIST1H2AB | 0.971115 | 250.2848 | 0.028885 | −2.75551 |
| SH3BGR | 0.9636 | 247.6998 | 0.0364 | −2.6753 |
| LENEP | 0.963524 | 244.7181 | 0.036476 | −2.82297 |
| INHBB | 0.976161 | 242.751 | 0.023839 | −2.88182 |
| IP6K1 | 0.994543 | 242.7268 | 0.005457 | −3.97994 |
| RPL39 | 0.997965 | 242.3342 | 0.002035 | −4.9625 |
| SF3B3 | 0.998132 | 242.0362 | 0.001868 | −4.86631 |
| DPYSL2 | 0.951864 | 240.6994 | 0.048136 | −2.74475 |
| VSIG4 | 0.999701 | 240.4777 | 0.000299 | −8.01084 |
| KLRC3 | 0.953387 | 240.0347 | 0.046613 | −2.56644 |
| POU3F1 | 0.970596 | 239.2105 | 0.029404 | −2.7798 |
| SCUBE1 | 0.99145 | 237.9207 | 0.00855 | −3.90924 |
| ITPA | 0.957636 | 236.6258 | 0.042364 | −2.47756 |
| BPHL | 0.953774 | 236.0256 | 0.046226 | −2.724 |
| EMILIN1 | 0.965148 | 235.9642 | 0.034852 | −2.67532 |
| MRPS34 | 0.988358 | 234.2746 | 0.011642 | −3.51791 |
| ATP5PO | 0.967922 | 234.1928 | 0.032078 | −2.69751 |
| PEX12 | 0.973966 | 232.5744 | 0.026034 | −3.82102 |
| CLEC4A | 0.987437 | 230.1604 | 0.012563 | −3.36201 |
| IGFBP2 | 0.96499 | 228.4802 | 0.03501 | −2.78347 |
| POU1F1 | 0.993731 | 228.1198 | 0.006269 | −4.51777 |
| RB1 | 0.992161 | 227.7552 | 0.007839 | −4.33366 |
| EWSR1 | 0.97092 | 226.2556 | 0.02908 | −2.73618 |
| JAML | 0.984112 | 225.4134 | 0.015888 | −3.44826 |
| ZNF98 | 0.97548 | 224.7575 | 0.02452 | −3.132 |
| MYL2 | 0.968275 | 222.56 | 0.031725 | −2.77654 |
| CDS1 | 0.986444 | 221.5158 | 0.013556 | −3.27635 |
| PLA2G6 | 0.987333 | 220.7842 | 0.012667 | −3.33671 |
| GGT2 | 0.953149 | 220.0928 | 0.046851 | −2.96097 |
| MYBL2 | 0.997046 | 220.0258 | 0.002954 | −4.51128 |
| SLC35E4 | 0.95516 | 218.7408 | 0.04484 | −2.55894 |
| TSPAN3 | 0.992839 | 218.1867 | 0.007161 | −4.3834 |
| FCGR2A | 0.998182 | 217.1938 | 0.001818 | −4.89805 |
| SOX18 | 0.995195 | 216.4809 | 0.004805 | −4.14928 |
| ECI2 | 0.996744 | 215.5611 | 0.003256 | −4.44448 |
| HNRNPK | 0.969053 | 215.4254 | 0.030947 | −2.83332 |
| OR2D3 | 0.997804 | 214.2403 | 0.002196 | −5.45535 |
| PCDHB3 | 0.991515 | 212.0395 | 0.008485 | −3.78722 |
| IGSF9 | 0.988806 | 210.647 | 0.011194 | −3.66407 |
| GALNT1 | 0.989401 | 210.6044 | 0.010599 | −3.46176 |
| LPA | 0.989297 | 210.1532 | 0.010703 | −3.80407 |
| PPFIA2 | 0.954538 | 209.93 | 0.045462 | −2.43138 |
| C3orf84 | 0.966378 | 209.6405 | 0.033622 | −2.84075 |
| PDCD6 | 0.974245 | 208.7203 | 0.025755 | −3.72364 |
| NTHL1 | 0.995609 | 207.2689 | 0.004391 | −4.21198 |
| CABLES2 | 0.953804 | 206.3498 | 0.046196 | −2.58312 |
| PRPF40A | 0.956533 | 205.9447 | 0.043467 | −3.11339 |
| ADGRG5 | 0.995043 | 205.4411 | 0.004957 | −4.33078 |
| HS6ST2 | 0.973406 | 204.971 | 0.026594 | −2.90784 |
| BCAT2 | 0.967452 | 202.8697 | 0.032548 | −2.70758 |
| CTSS | 0.968367 | 202.7172 | 0.031633 | −2.89757 |
| CAPN7 | 0.95329 | 202.5098 | 0.04671 | −2.42045 |
| SFTA2 | 0.984863 | 202.3885 | 0.015137 | −3.25072 |
| DAPK2 | 0.998537 | 202.2543 | 0.001463 | −5.72225 |
| OR5B12 | 0.966099 | 199.0872 | 0.033901 | −2.68734 |
| SLC12A9 | 0.990117 | 197.5187 | 0.009883 | −3.74945 |
| THSD1 | 0.956922 | 196.5949 | 0.043078 | −2.61403 |
| SLC26A9 | 0.968311 | 195.8944 | 0.031689 | −2.9452 |
| ZNF266 | 0.999967 | 192.9796 | 3.32E−05 | −9.5869 |
| RPE65 | 0.97417 | 191.6234 | 0.02583 | −3.41796 |
| SEPHS2 | 0.959546 | 191.489 | 0.040454 | −2.52822 |
| ABCB9 | 0.970569 | 189.1783 | 0.029431 | −3.09766 |
| E2F1 | 0.967929 | 188.8396 | 0.032071 | −2.69113 |
| HSF1 | 0.951722 | 187.3413 | 0.048278 | −2.89146 |
| MFRP | 0.99386 | 184.9856 | 0.00614 | −5.69141 |
| SLC2A7 | 0.986561 | 183.8784 | 0.013439 | −3.47976 |
| TCEA2 | 0.983511 | 183.2386 | 0.016489 | −4.1519 |
| ZNF257 | 0.99315 | 181.7744 | 0.00685 | −3.85184 |
| NPIPB13 | 0.980389 | 181.6733 | 0.019611 | −3.66617 |
| MAP3K10 | 0.992907 | 180.0278 | 0.007093 | −3.96453 |
| SERPINH1 | 0.959103 | 177.4504 | 0.040897 | −2.56758 |
| VCAN | 0.956853 | 176.689 | 0.043147 | −2.93952 |
| NCAPH | 0.990805 | 176.431 | 0.009195 | −4.08551 |
| INPP5B | 0.996521 | 176.2881 | 0.003479 | −4.33429 |
| ATG5 | 0.99825 | 176.2094 | 0.00175 | −4.9831 |
| CYP2A7 | 0.968853 | 175.4719 | 0.031147 | −2.78709 |
| RRS1 | 0.989252 | 175.4399 | 0.010748 | −3.59703 |
| PCDHGA4 | 0.982799 | 173.7935 | 0.017201 | −3.15375 |
| TXK | 0.962419 | 173.6765 | 0.037581 | −2.72992 |
| GCLM | 0.968787 | 171.5189 | 0.031213 | −3.15377 |
| MAP3K4 | 0.990441 | 171.3835 | 0.009559 | −4.04564 |
| CLTA | 0.980307 | 171.2061 | 0.019693 | −3.09497 |
| RASAL1 | 0.978702 | 170.8835 | 0.021298 | −2.95497 |
| SSX1 | 0.990143 | 170.8606 | 0.009857 | −3.81923 |
| RNF114 | 0.972184 | 169.9093 | 0.027816 | −3.03521 |
| HTR4 | 0.997275 | 169.2366 | 0.002725 | −4.88112 |
| NPAP1 | 0.956283 | 168.1742 | 0.043717 | −2.46247 |
| DEDD | 0.974686 | 167.9191 | 0.025314 | −2.8998 |
| WNT5A | 0.978131 | 166.5247 | 0.021869 | −3.3663 |
| FOXA3 | 0.951672 | 165.7271 | 0.048328 | −2.75841 |
| CEP83 | 0.985561 | 164.9226 | 0.014439 | −3.61989 |
| TRPM8 | 0.987078 | 164.4172 | 0.012922 | −4.17523 |
| PSMA3 | 0.977091 | 164.0135 | 0.022909 | −2.91784 |
| CNOT1 | 0.968153 | 163.9039 | 0.031847 | −2.94916 |
| USP9X | 0.975992 | 163.8317 | 0.024008 | −3.17804 |
| KRTAP9-6 | 0.992787 | 162.8298 | 0.007213 | −3.76866 |
| NUP133 | 0.985062 | 161.5868 | 0.014938 | −3.26441 |
| GRK3 | 0.972846 | 161.3808 | 0.027154 | −2.78586 |
| OR6K3 | 0.965489 | 161.1397 | 0.034511 | −2.76306 |
| PRIMA1 | 0.993054 | 161.1165 | 0.006946 | −3.86065 |
| AMPD2 | 0.979146 | 159.9865 | 0.020854 | −4.90974 |
| THUMPD2 | 0.958488 | 159.2197 | 0.041512 | −2.49196 |
| WRAP73 | 0.953126 | 157.4406 | 0.046874 | −2.41111 |
| TTC4 | 0.962358 | 157.2872 | 0.037642 | −2.7561 |
| RAB41 | 0.964715 | 156.8134 | 0.035285 | −3.18298 |
| DBN1 | 0.964122 | 155.4798 | 0.035878 | −2.66168 |
| ZAP70 | 0.967178 | 154.7173 | 0.032822 | −2.99223 |
| GJB3 | 0.970143 | 154.4417 | 0.029857 | −3.31828 |
| MT-ATP8 | 0.965132 | 154.3215 | 0.034868 | −2.68065 |
| GALNT17 | 0.990346 | 153.2926 | 0.009654 | −3.52631 |
| ATP5F1E | 0.958506 | 151.791 | 0.041494 | −2.58039 |
| LSM10 | 0.955165 | 151.4472 | 0.044835 | −2.56315 |
| KIF22 | 0.958993 | 151.0157 | 0.041007 | −2.73512 |
| EPHA1 | 0.986577 | 150.1858 | 0.013423 | −3.29569 |
| ARMT1 | 0.952546 | 149.5214 | 0.047454 | −2.80659 |
| GFPT1 | 0.997039 | 148.5274 | 0.002961 | −4.68985 |
| VEGFD | 0.974832 | 148.4167 | 0.025168 | −2.85754 |
| PRICKLE4 | 0.966694 | 147.8554 | 0.033306 | −2.74508 |
| SLCO4A1 | 0.99481 | 147.0142 | 0.00519 | −4.71514 |
| NOLC1 | 0.994499 | 145.2143 | 0.005501 | −4.38335 |
| NOP14 | 0.957352 | 144.7352 | 0.042648 | −2.51663 |
| DFFA | 0.996605 | 144.3267 | 0.003395 | −4.34605 |
| PRKRA | 0.981796 | 142.1364 | 0.018204 | −3.0742 |
| LHFPL1 | 0.992248 | 141.8882 | 0.007752 | −3.69058 |
| OR2H1 | 0.996584 | 141.1996 | 0.003416 | −4.37955 |
| TAAR2 | 0.993015 | 140.8381 | 0.006985 | −3.82025 |
| SOCS5 | 0.961016 | 140.6135 | 0.038984 | −2.54017 |
| PCDHB12 | 0.992738 | 138.85 | 0.007262 | −4.11447 |
| ADCY9 | 0.988684 | 138.0827 | 0.011316 | −3.41596 |
| C6orf15 | 0.992465 | 137.336 | 0.007535 | −4.2123 |
| OR51A7 | 0.971561 | 136.8124 | 0.028439 | −2.75334 |
| TBC1D3F | 0.963579 | 136.8083 | 0.036421 | −2.89587 |
| VPREB3 | 0.991815 | 136.6253 | 0.008185 | −3.65132 |
| SIRT4 | 0.978133 | 135.5158 | 0.021867 | −3.03033 |
| PNMA5 | 0.995516 | 134.5379 | 0.004484 | −4.60269 |
| PDE6A | 0.993792 | 130.4885 | 0.006208 | −4.20619 |
| LPAR2 | 0.980254 | 130.2751 | 0.019746 | −3.01098 |
| PRODH | 0.976591 | 130.2088 | 0.023409 | −3.06781 |
| ACOX1 | 0.985681 | 130.1329 | 0.014319 | −3.74745 |
| SNORA68 | 0.983524 | 129.5142 | 0.016476 | −3.15023 |
| PDZRN3 | 0.972365 | 128.7088 | 0.027635 | −2.91794 |
| ZNF311 | 0.997886 | 125.4051 | 0.002114 | −5.12034 |
| LRRC14B | 0.991872 | 123.7506 | 0.008128 | −3.65497 |
| NRSN2 | 0.960831 | 123.7156 | 0.039169 | −2.65092 |
| KAT14 | 0.973344 | 123.3422 | 0.026656 | −3.04422 |
| CX3CL1 | 0.970616 | 122.662 | 0.029384 | −2.91034 |
| TGFBR3 | 0.98397 | 122.2896 | 0.01603 | −3.41212 |
| TFAP2B | 0.960539 | 121.2723 | 0.039461 | −2.57346 |
| KCNJ14 | 0.953174 | 120.3812 | 0.046826 | −2.55885 |
| APC | 0.990354 | 119.1797 | 0.009646 | −3.66246 |
| TMPRSS4 | 0.971116 | 119.0388 | 0.028884 | −2.74626 |
| ZNF160 | 0.955129 | 117.562 | 0.044871 | −2.64121 |
| TTC8 | 0.984926 | 117.0735 | 0.015074 | −3.26017 |
| AIPL1 | 0.962373 | 115.4033 | 0.037627 | −2.75149 |
| SASH3 | 0.98329 | 114.9273 | 0.01671 | −3.13372 |
| EXOSC4 | 0.953253 | 114.8535 | 0.046747 | −2.4124 |
| ABCA6 | 0.957248 | 113.9417 | 0.042752 | −2.667 |
| ATP5MGL | 0.956316 | 113.5951 | 0.043684 | −2.54235 |
| IMP3 | 0.98712 | 113.1757 | 0.01288 | −3.58759 |
| FZD4 | 0.987323 | 112.7941 | 0.012677 | −3.47607 |
| DRD4 | 0.970189 | 112.4363 | 0.029811 | −2.72506 |
| COLEC10 | 0.951148 | 112.1126 | 0.048852 | −2.48996 |
| ACVR2B | 0.974747 | 111.9894 | 0.025253 | −3.34929 |
| NDUFB1 | 0.979102 | 111.8837 | 0.020898 | −3.11114 |
| ZFP36L2 | 0.979297 | 109.2595 | 0.020703 | −2.98125 |
| KLK12 | 0.984708 | 108.386 | 0.015292 | −3.21903 |
| GABRA5 | 0.977025 | 108.1499 | 0.022975 | −2.9559 |
| SLC30A5 | 0.98758 | 107.6792 | 0.01242 | −3.34465 |
| TNRC6A | 0.96082 | 106.6962 | 0.03918 | −2.63142 |
| LYN | 0.998207 | 106.112 | 0.001793 | −4.88082 |
| TPSAB1 | 0.984281 | 105.436 | 0.015719 | −3.23337 |
| OR2AG1 | 0.953663 | 105.4293 | 0.046337 | −2.84122 |
| UBA52 | 0.977435 | 105.1724 | 0.022565 | −2.9719 |
| PLXNA1 | 0.960139 | 104.491 | 0.039861 | −2.59871 |
| ACADL | 0.975826 | 103.6936 | 0.024174 | −3.01647 |
| UBE2K | 0.999664 | 103.4719 | 0.000336 | −6.69585 |
| PKIG | 0.975576 | 102.3314 | 0.024424 | −3.04316 |
| MED15 | 0.966307 | 102.0066 | 0.033693 | −2.69363 |
| TMX1 | 0.970359 | 101.7031 | 0.029641 | −2.81259 |
| NR5A1 | 0.959679 | 101.6825 | 0.040321 | −2.79775 |
| DEPDC5 | 0.95349 | 101.5396 | 0.04651 | −2.54321 |
| MYPOP | 0.990527 | 101.3547 | 0.009473 | −3.67441 |
| ABCD4 | 0.968658 | 101.1586 | 0.031342 | −2.93434 |
| JPH4 | 0.993789 | 100.8319 | 0.006211 | −4.11021 |
| KIRREL1 | 0.987631 | 100.2713 | 0.012369 | −3.43664 |
| MYO15A | 0.997714 | 100.1618 | 0.002286 | −5.50485 |
| LSS | 0.975875 | 99.01838 | 0.024125 | −3.15523 |
| LARS | 0.995827 | 98.93125 | 0.004173 | −4.60496 |
| GPN1 | 0.980991 | 98.66719 | 0.019009 | −3.09142 |
| ZBTB17 | 0.974245 | 98.54073 | 0.025755 | −2.83176 |
| VANGL1 | 0.956981 | 98.49528 | 0.043019 | −2.61493 |
| SPTBN1 | 0.962293 | 98.47626 | 0.037707 | −2.97051 |
| TMOD1 | 0.994742 | 97.55208 | 0.005258 | −4.13829 |
| LRRC4 | 0.963017 | 97.3971 | 0.036983 | −2.61612 |
| ZBTB49 | 0.96143 | 97.30108 | 0.03857 | −3.46859 |
| CST6 | 0.993927 | 97.14576 | 0.006073 | −4.03954 |
| DGUOK | 0.980999 | 96.34867 | 0.019001 | −3.22481 |
| CNOT7 | 0.967618 | 95.88511 | 0.032382 | −4.14575 |
| RGS5 | 0.983247 | 94.86668 | 0.016753 | −3.12428 |
| SLIRP | 0.986538 | 94.71965 | 0.013462 | −3.33241 |
| MAGEA9 | 0.997986 | 94.57542 | 0.002014 | −5.08258 |
| MYC | 0.96392 | 93.58858 | 0.03608 | −3.48447 |
| CASC3 | 0.951059 | 93.32643 | 0.048941 | −2.37933 |
| CLDN15 | 0.994721 | 92.46355 | 0.005279 | −4.28477 |
| MAN1B1 | 0.963599 | 91.8295 | 0.036401 | −2.62065 |
| HOXB6 | 0.979558 | 91.80079 | 0.020442 | −2.98909 |
| C4B | 0.97654 | 91.75679 | 0.02346 | −3.00671 |
| KDM5B | 0.97725 | 91.59727 | 0.02275 | −2.91348 |
| CACNB3 | 0.95301 | 91.08143 | 0.04699 | −2.46619 |
| ANPEP | 0.959679 | 90.66731 | 0.040321 | −2.74676 |
| SFXN2 | 0.98082 | 90.59044 | 0.01918 | −3.11239 |
| CSRP3 | 0.958391 | 90.31043 | 0.041609 | −2.56187 |
| DLEU2 | 0.966175 | 90.29044 | 0.033825 | −2.82036 |
| GBP1 | 0.950379 | 90.10444 | 0.049621 | −2.37674 |
| RPLP1 | 0.996311 | 89.96278 | 0.003689 | −4.34469 |
| ARID5B | 0.984599 | 87.26793 | 0.015401 | −4.41847 |
| CHAT | 0.984464 | 86.94869 | 0.015536 | −3.17842 |
| INE1 | 0.997892 | 86.45082 | 0.002108 | −5.00053 |
| SLC5A8 | 0.976392 | 85.85185 | 0.023608 | −2.9548 |
| CAMK4 | 0.999927 | 85.5679 | 7.3E−05 | −8.31126 |
| SPO11 | 0.996335 | 85.5134 | 0.003665 | −4.44813 |
| OR6X1 | 0.981928 | 85.44099 | 0.018072 | −3.92317 |
| VSNL1 | 0.996462 | 85.40089 | 0.003538 | −4.5444 |
| ACR | 0.970939 | 84.16234 | 0.029061 | −2.96526 |
| SLC4A8 | 0.966684 | 83.1787 | 0.033316 | −2.6534 |
| IKZF5 | 0.97629 | 82.11526 | 0.02371 | −2.96982 |
| ACOT11 | 0.988941 | 81.34844 | 0.011059 | −3.42516 |
| DCXR | 0.990131 | 81.18014 | 0.009869 | −3.57743 |
| ARHGEF11 | 0.955192 | 80.31437 | 0.044808 | −2.50365 |
| USP49 | 0.959919 | 79.44248 | 0.040081 | −2.51544 |
| LALBA | 0.970818 | 79.29405 | 0.029182 | −2.73986 |
| BMP8B | 0.99157 | 78.40238 | 0.00843 | −3.64048 |
| RPS24 | 0.98409 | 78.13809 | 0.01591 | −3.16683 |
| SMYD3 | 0.982609 | 77.39647 | 0.017391 | −3.1659 |
| SREBF2 | 0.961995 | 77.34376 | 0.038005 | −2.77761 |
| SERPINA5 | 0.973316 | 77.14271 | 0.026684 | −2.83621 |
| SHMT2 | 0.972648 | 76.7435 | 0.027352 | −2.79571 |
| STX17 | 0.991917 | 76.24455 | 0.008083 | −3.66174 |
| LY96 | 0.993309 | 75.99197 | 0.006691 | −3.89167 |
| CACNG4 | 0.962015 | 75.95724 | 0.037985 | −3.04157 |
| RNF7 | 0.955825 | 75.61798 | 0.044175 | −2.63545 |
| CIC | 0.991616 | 75.21341 | 0.008384 | −3.66346 |
| PFDN2 | 0.955381 | 75.06853 | 0.044619 | −2.49689 |
| FKBP9 | 0.961678 | 75.01903 | 0.038322 | −2.6198 |
| ZBTB1 | 0.99279 | 73.05578 | 0.00721 | −3.93091 |
| ADAM3A | 0.976471 | 72.52161 | 0.023529 | −2.90034 |
| MRVI1 | 0.975194 | 72.06118 | 0.024806 | −3.37788 |
| MAN1B1-DT | 0.992149 | 70.59436 | 0.007851 | −3.84155 |
| UCK1 | 0.973941 | 70.3499 | 0.026059 | −4.12231 |
| PRDX4 | 0.960039 | 70.05581 | 0.039961 | −2.51835 |
| ORC2 | 0.99843 | 69.29319 | 0.00157 | −6.85355 |
| HIST1H4H | 0.959565 | 69.22542 | 0.040435 | −2.53447 |
| TAF8 | 0.982842 | 68.9089 | 0.017158 | −3.51408 |
| ZFPL1 | 0.990014 | 68.59953 | 0.009986 | −3.87101 |
| ZNF408 | 0.965382 | 68.53386 | 0.034618 | −2.65597 |
| SIGLEC7 | 0.968056 | 68.12911 | 0.031944 | −2.70204 |
| HIBCH | 0.982742 | 68.06499 | 0.017258 | −3.12164 |
| NRXN1 | 0.987065 | 67.46987 | 0.012935 | −3.4164 |
| RBL1 | 0.985894 | 67.45643 | 0.014106 | −3.27714 |
| PTK2 | 0.987147 | 67.36314 | 0.012853 | −3.42301 |
| GLRA3 | 0.967926 | 66.66893 | 0.032074 | −2.95467 |
| PRPS1 | 0.960214 | 66.65729 | 0.039786 | −2.74201 |
| MRPS10 | 0.993437 | 65.27115 | 0.006563 | −3.8228 |
| OR1G1 | 0.987882 | 65.17822 | 0.012118 | −3.60951 |
| KCNQ4 | 0.996796 | 63.19014 | 0.003204 | −4.65266 |
| CD300C | 0.999597 | 62.83685 | 0.000403 | −8.50605 |
| PCDHGC5 | 0.990081 | 62.8323 | 0.009919 | −3.5168 |
| CLEC11A | 0.976212 | 62.7796 | 0.023788 | −2.99258 |
| ARPC4 | 0.990871 | 61.93385 | 0.009129 | −3.77328 |
| KRT38 | 0.958293 | 61.90526 | 0.041707 | −2.4894 |
| GNPAT | 0.955348 | 61.78511 | 0.044652 | −2.8823 |
| CRIP1 | 0.950377 | 61.65479 | 0.049623 | −2.55562 |
| TMEM229A | 0.962946 | 60.09181 | 0.037054 | −2.57368 |
| RASGRP2 | 0.97735 | 58.70816 | 0.02265 | −2.95062 |
| TRIP10 | 0.967166 | 58.10985 | 0.032834 | −3.69877 |
| THOC2 | 0.999821 | 57.73357 | 0.000179 | −7.51658 |
| CKLF- | 0.98767 | 57.72078 | 0.01233 | −3.37129 |
| CMTM1 | | | | |
| CGB1 | 0.979415 | 57.68987 | 0.020585 | −3.21562 |
| RECQL5 | 0.982555 | 57.26549 | 0.017445 | −3.24701 |
| MSI2 | 0.996452 | 57.16671 | 0.003548 | −4.73219 |
| INSR | 0.959897 | 55.35392 | 0.040103 | −2.77555 |
| UGT2B10 | 0.958245 | 54.99102 | 0.041755 | −2.5813 |
| HNRNPA1 | 0.9835 | 54.71765 | 0.0165 | −3.62019 |
| HFM1 | 0.990255 | 54.51508 | 0.009745 | −3.62699 |
| PSMA5 | 0.980735 | 54.44022 | 0.019265 | −3.04514 |
| EYA2 | 0.979619 | 53.78134 | 0.020381 | −2.98815 |
| PPP2R2A | 0.971717 | 53.5782 | 0.028283 | −3.03798 |
| RPS7 | 0.999914 | 53.08532 | 8.55E−05 | −8.89992 |
| GAPDH | 0.957786 | 51.67144 | 0.042214 | −2.48984 |
| CRNN | 0.955064 | 51.31582 | 0.044936 | −2.5217 |
| NTNG2 | 0.95766 | 49.74424 | 0.04234 | −3.03159 |
| EOMES | 0.974454 | 49.6261 | 0.025546 | −3.0851 |
| DDX41 | 0.960168 | 48.90857 | 0.039832 | −2.559 |
| DPF3 | 0.959165 | 48.24591 | 0.040835 | −2.52001 |
| ADRA2B | 0.98179 | 47.7761 | 0.01821 | −3.26252 |
| TPCN1 | 0.977413 | 46.81778 | 0.022587 | −2.9327 |
| KRT7 | 0.973189 | 46.70192 | 0.026811 | −3.42878 |
| SGPL1 | 0.978794 | 46.44698 | 0.021206 | −3.05565 |
| FAM120C | 0.95781 | 45.52517 | 0.04219 | −3.46776 |
| GPBP1L1 | 0.972712 | 45.14957 | 0.027288 | −2.78043 |
| COL4A5 | 0.958524 | 44.99846 | 0.041476 | −2.50879 |
| WFS1 | 0.99209 | 44.9563 | 0.00791 | −3.89894 |
| OR4F21 | 0.953368 | 44.01311 | 0.046632 | −2.55944 |
| OR2A12 | 0.978076 | 43.96031 | 0.021924 | −3.02553 |
| DMRT1 | 0.980549 | 43.76572 | 0.019451 | −3.6764 |
| ATP6V1B2 | 0.996723 | 43.19108 | 0.003277 | −4.85864 |
| SRP72 | 0.961129 | 42.98105 | 0.038871 | −2.5407 |
| CCDC25 | 0.961823 | 42.43598 | 0.038177 | −2.55294 |
| PIM1 | 0.965233 | 42.13152 | 0.034767 | −2.73471 |
| KCNE5 | 0.992291 | 42.02449 | 0.007709 | −3.69587 |
| DOCK9 | 0.955169 | 41.63304 | 0.044831 | −2.4848 |
| HIST3H3 | 0.985869 | 41.34182 | 0.014131 | −3.32996 |
| CTSD | 0.993828 | 40.32086 | 0.006172 | −3.86847 |
| AKAP3 | 0.968839 | 40.16332 | 0.031161 | −2.71851 |
| SPRR2E | 0.950918 | 40.14844 | 0.049082 | −2.46566 |
| FADS3 | 0.968365 | 38.00026 | 0.031635 | −2.76648 |
| HOXB13 | 0.956764 | 37.3464 | 0.043236 | −2.47896 |
| SLC23A1 | 0.970628 | 36.507 | 0.029372 | −2.89401 |
| GJA4 | 0.976195 | 36.37219 | 0.023805 | −2.95914 |
| SLC24A3 | 0.964968 | 36.31537 | 0.035032 | −2.70151 |
| PTTG1IP | 0.966081 | 36.0532 | 0.033919 | −2.74037 |
| GUCY2D | 0.962878 | 35.53088 | 0.037122 | −2.58044 |
| CA10 | 0.988622 | 35.44853 | 0.011378 | −3.44057 |
| CAPG | 0.959384 | 34.95108 | 0.040616 | −2.51318 |
| LFNG | 0.982963 | 34.74352 | 0.017037 | −3.12066 |
| SERPIND1 | 0.966347 | 33.73193 | 0.033653 | −2.70804 |
| OPN1LW | 0.980357 | 33.61336 | 0.019643 | −3.10676 |
| NUMA1 | 0.960944 | 33.472 | 0.039056 | −2.61122 |
| RAB34 | 0.957031 | 33.36563 | 0.042969 | −2.52812 |
| RACGAP1 | 0.974573 | 33.19858 | 0.025427 | −2.97309 |
| SRC | 0.986021 | 32.95772 | 0.013979 | −3.26551 |
| PTN | 0.966692 | 32.12163 | 0.033308 | −2.8109 |
| HCRT | 0.995213 | 32.03095 | 0.004787 | −4.25694 |
| TIMP3 | 0.964694 | 31.55441 | 0.035306 | −3.50551 |
| SGCD | 0.95403 | 31.54754 | 0.04597 | −2.48753 |
| FEZ2 | 0.99204 | 31.4019 | 0.00796 | −3.98392 |
| PML | 0.95905 | 31.33623 | 0.04095 | −2.67314 |
| SH3BGRL2 | 0.961453 | 30.82544 | 0.038547 | −2.73534 |
| INAFM2 | 0.978958 | 30.03562 | 0.021042 | −3.80629 |
| ZNF75A | 0.966821 | 29.92257 | 0.033179 | −3.24256 |
| AIF1 | 0.953171 | 29.38309 | 0.046829 | −2.44285 |
| ZNF792 | 0.953679 | 27.40733 | 0.046321 | −2.44937 |
| EXT1 | 0.998181 | 27.31023 | 0.001819 | −4.87166 |
| MRPS28 | 0.969617 | 27.29214 | 0.030383 | −2.72653 |
| CTNNA1 | 0.987017 | 27.20455 | 0.012983 | −3.503 |
| GC | 0.956512 | 27.04852 | 0.043488 | −2.50384 |
| MALL | 0.995083 | 26.87454 | 0.004917 | −6.71522 |
| CYB5A | 0.990246 | 26.81455 | 0.009754 | −3.66615 |
| PCDHGA8 | 0.979821 | 26.1231 | 0.020179 | −3.15795 |
| GH1 | 0.95416 | 25.84482 | 0.04584 | −2.62123 |
| GDAP1L1 | 0.963173 | 24.62556 | 0.036827 | −2.78812 |
| BTBD3 | 0.986957 | 24.45572 | 0.013043 | −3.93504 |
| MRPL2 | 0.972896 | 24.28161 | 0.027104 | −3.08199 |
| OR5D18 | 0.970291 | 23.74422 | 0.029709 | −2.83113 |
| CCDC73 | 0.988917 | 23.09812 | 0.011083 | −6.49941 |
| RASSF5 | 0.972213 | 22.97823 | 0.027787 | −2.88268 |
| PAK3 | 0.987913 | 21.46507 | 0.012087 | −3.43163 |
| ABCA7 | 0.994499 | 21.21039 | 0.005501 | −4.8973 |
| FEV | 0.996151 | 21.05581 | 0.003849 | −4.59096 |
| CXCL13 | 0.999593 | 21.03922 | 0.000407 | −6.38747 |
| REXO4 | 0.979039 | 20.5391 | 0.020961 | −3.14307 |
| TBXAS1 | 0.983325 | 19.30239 | 0.016675 | −3.21038 |
| COL23A1 | 0.98983 | 18.8439 | 0.01017 | −4.15126 |
| TMPRSS2 | 0.981408 | 17.66938 | 0.018592 | −3.04977 |
| ADGRE1 | 0.955217 | 17.34884 | 0.044783 | −3.00748 |
| OPTC | 0.969258 | 16.9337 | 0.030742 | −3.18655 |
| BICD1 | 0.952536 | 16.61637 | 0.047464 | −2.59634 |
| OR52E6 | 0.969831 | 16.59107 | 0.030169 | −2.7359 |
| EPDR1 | 0.96515 | 16.51958 | 0.03485 | −3.45278 |
| DERL2 | 0.960611 | 16.49175 | 0.039389 | −2.5837 |
| ZNF335 | 0.969703 | 16.11395 | 0.030297 | −2.7235 |
| LGALS16 | 0.991306 | 16.05182 | 0.008694 | −3.60401 |
| PPP3CC | 0.961978 | 15.9475 | 0.038022 | −2.59952 |
| ZNF177 | 0.988725 | 15.58303 | 0.011275 | −3.46888 |
| HIST1H1D | 0.996838 | 14.6902 | 0.003162 | −4.57975 |
| SRSF9 | 0.990956 | 14.65572 | 0.009044 | −3.61047 |
| CHRD | 0.969448 | 14.36303 | 0.030552 | −2.82817 |
| CNIH3 | 0.979855 | 14.31071 | 0.020145 | −2.99481 |
| MNDA | 0.956057 | 14.2222 | 0.043943 | −2.45286 |
| KRTAP5-10 | 0.966413 | 14.18911 | 0.033587 | −4.73702 |
| UBE2Q2 | 0.954409 | 14.05085 | 0.045591 | −2.4489 |
| ANKRD18A | 0.976322 | 13.84432 | 0.023678 | −2.96698 |
| GSTK1 | 0.997491 | 13.76717 | 0.002509 | −4.65355 |
| PTEN | 0.991591 | 13.04732 | 0.008409 | −3.70572 |
| INHA | 0.995717 | 12.32834 | 0.004283 | −6.07436 |
| MIPEP | 0.971775 | 12.14757 | 0.028225 | −2.81589 |
| WDR17 | 0.955917 | 11.98304 | 0.044083 | −2.53974 |
| CDHR5 | 0.961153 | 11.58986 | 0.038847 | −2.85716 |
| ANGPTL6 | 0.996445 | 11.03758 | 0.003555 | −4.31518 |
| ASB6 | 0.993535 | 9.777699 | 0.006465 | −4.15507 |
| DSN1 | 0.977068 | 9.546554 | 0.022932 | −3.79459 |
| KRT14 | 0.961636 | 9.245878 | 0.038364 | −3.57596 |
| COX7C | 0.983182 | 8.959501 | 0.016818 | −3.98318 |
| AOPEP | 0.988848 | 8.824513 | 0.011152 | −3.60891 |
| ZNF121 | 0.96279 | 8.379351 | 0.03721 | −2.66148 |
| PEBP1 | 0.965094 | 8.196101 | 0.034906 | −2.65242 |
| MR1 | 0.998111 | 8.052685 | 0.001889 | −5.15938 |
| KDELR3 | 0.983703 | 8.036852 | 0.016297 | −3.15086 |
| AGER | 0.955103 | 8.008272 | 0.044897 | −2.46977 |
| PITPNB | 0.967506 | 7.999911 | 0.032494 | −2.67089 |
| TBC1D16 | 0.986616 | 7.641421 | 0.013384 | −3.41054 |
| APH1B | 0.969175 | 7.255681 | 0.030825 | −3.01603 |
| GLIS3 | 0.976383 | 7.071892 | 0.023617 | −2.92796 |
| SMARCC1 | 0.995507 | 6.850142 | 0.004493 | −4.37145 |
| PCNX3 | 0.957768 | 6.802188 | 0.042232 | −2.78044 |
| GGCX | 0.987772 | 6.709272 | 0.012228 | −4.07523 |
| SLC43A2 | 0.990012 | 6.58801 | 0.009988 | −3.70874 |
| OR4D10 | 0.975519 | 6.299446 | 0.024481 | −2.88078 |
| IFT57 | 0.97085 | 6.22636 | 0.02915 | −2.95335 |
| TDRG1 | 0.987535 | 6.150395 | 0.012465 | −4.24828 |
| TAF15 | 0.998419 | 5.97294 | 0.001581 | −5.31354 |
| CIZ1 | 0.990847 | 5.770671 | 0.009153 | −3.65599 |
| H1FOO | 0.985074 | 5.367482 | 0.014926 | −3.4165 |
| HAO1 | 0.989589 | 4.41629 | 0.010411 | −3.51798 |
| SEBOX | 0.956834 | 4.15292 | 0.043166 | −3.20809 |
| KRT73 | 0.956088 | 3.935305 | 0.043912 | −4.28441 |
| MICU1 | 0.983605 | 3.695701 | 0.016395 | −4.25972 |
| OSBPL1A | 0.967783 | 3.681905 | 0.032217 | −4.82475 |
| HOXA3 | 0.966344 | 3.66413 | 0.033656 | −2.81726 |
| PCDHGA3 | 0.964431 | 3.491053 | 0.035569 | −2.7537 |
| IDE | 0.973122 | 3.438656 | 0.026878 | −2.79342 |
| PTH | 0.986308 | 3.437431 | 0.013692 | −3.42003 |
| HMHB1 | 0.983735 | 3.356401 | 0.016265 | −3.1765 |
| GBP7 | 0.954283 | 3.286571 | 0.045717 | −2.55815 |
| IQCF3 | 0.979728 | 3.090712 | 0.020272 | −3.89123 |
| C19orf48 | 0.967174 | 2.884688 | 0.032826 | −2.80703 |
| CASZ1 | 0.963912 | 2.847923 | 0.036088 | −3.27363 |
| TOMM7 | 0.993889 | 2.708066 | 0.006111 | −12.7338 |
| HYPM | 0.994774 | 2.63076 | 0.005226 | −4.19751 |
| TMPRSS13 | 0.974839 | 2.274724 | 0.025161 | −4.69495 |
| HLA-DRB6 | 0.952857 | 2.262711 | 0.047143 | −4.44119 |
| PODN | 0.956995 | 2.04499 | 0.043005 | −2.47302 |
| GSDME | 0.965007 | 1.930444 | 0.034993 | −5.20446 |
| YIPF2 | 0.961758 | 1.780234 | 0.038242 | −2.71189 |
| ACSL3 | 0.975156 | 1.759212 | 0.024844 | −4.30612 |
| DHX29 | 0.999606 | 1.306105 | 0.000394 | −6.73182 |
| ZKSCAN2 | 0.967337 | 1.222978 | 0.032663 | −5.39663 |
| ZFAND6 | 0.967337 | 1.222978 | 0.032663 | −5.39663 |
| RPS4Y1 | 0.967337 | 1.222978 | 0.032663 | −5.39663 |
| TERB1 | 0.957908 | 1.19777 | 0.042092 | −2.54529 |
| COMMD10 | 0.975352 | 1.127123 | 0.024648 | −3.08196 |
| MRGBP | 0.978922 | 1.004208 | 0.021078 | −3.18811 |
| CCDC90B | 0.044192 | −1.28189 | 0.044192 | 2.672948 |
| POTEJ | 0.029946 | −1.62174 | 0.029946 | 3.004431 |
| ZNF682 | 0.037039 | −1.7432 | 0.037039 | 2.821586 |
| AGTRAP | 0.045219 | −1.75529 | 0.045219 | 2.653809 |
| HSCB | 0.030015 | −1.83697 | 0.030015 | 3.002438 |
| C16orf95 | 0.041818 | −1.88739 | 0.041818 | 2.555451 |
| SMIM10L2B | 0.02197 | −2.65059 | 0.02197 | 3.279342 |
| CFAP53 | 0.031365 | −3.27849 | 0.031365 | 2.786246 |
| CNTLN | 0.034548 | −3.45441 | 0.034548 | 2.699525 |
| LHFPL6 | 0.024618 | −3.77787 | 0.024618 | 2.991027 |
| SH3RF2 | 0.038524 | −4.91988 | 0.038524 | 2.560779 |
| SOD3 | 0.020834 | −5.29036 | 0.020834 | 2.986102 |
| RASSF4 | 0.028508 | −5.45014 | 0.028508 | 2.760465 |
| MBD4 | 0.027971 | −5.78811 | 0.027971 | 2.832509 |
| UBXN10 | 0.033407 | −5.9703 | 0.033407 | 2.69068 |
| SLC25A45 | 0.043147 | −6.59992 | 0.043147 | 2.692938 |
| GRXCR2 | 0.034645 | −7.02982 | 0.034645 | 2.676589 |
| ADD2 | 0.043407 | −7.03143 | 0.043407 | 2.546774 |
| PARD3 | 0.023648 | −7.03607 | 0.023648 | 3.061993 |
| LUZP1 | 0.035352 | −7.56596 | 0.035352 | 2.842674 |
| SULT1A2 | 0.040936 | −7.62084 | 0.040936 | 2.59356 |
| ADRA2C | 0.039036 | −7.85854 | 0.039036 | 2.553077 |
| CYP4F11 | 0.002104 | −7.87746 | 0.002104 | 4.928065 |
| NAE1 | 0.036294 | −8.23597 | 0.036294 | 2.734063 |
| MIR210 | 0.041204 | −8.31154 | 0.041204 | 2.670876 |
| ITCH | 0.017546 | −8.34972 | 0.017546 | 3.094313 |
| CHST12 | 0.032607 | −8.51737 | 0.032607 | 2.865027 |
| DCANP1 | 0.026322 | −8.67678 | 0.026322 | 2.805481 |
| DGAT2L6 | 0.032296 | −8.74833 | 0.032296 | 2.719997 |
| ZNF271P | 0.043866 | −9.24053 | 0.043866 | 2.535627 |
| OLFM3 | 0.027272 | −9.3868 | 0.027272 | 2.827862 |
| SEMA6C | 0.047451 | −9.57893 | 0.047451 | 2.424879 |
| MCMBP | 0.039111 | −9.84393 | 0.039111 | 2.665849 |
| ENDOD1 | 0.041737 | −10.4339 | 0.041737 | 2.577169 |
| GZF1 | 0.035707 | −10.4575 | 0.035707 | 2.641932 |
| ANXA7 | 0.040497 | −10.5222 | 0.040497 | 2.640794 |
| TNFAIP8L1 | 0.02581 | −10.7912 | 0.02581 | 2.819028 |
| CLDN6 | 0.007992 | −11.3276 | 0.007992 | 4.224623 |
| XXYLT1 | 0.011914 | −11.5492 | 0.011914 | 4.548596 |
| PCLO | 0.045621 | −11.6224 | 0.045621 | 2.461052 |
| ZNRF4 | 0.030756 | −11.6568 | 0.030756 | 2.78586 |
| KCNH8 | 0.034156 | −11.9952 | 0.034156 | 2.966237 |
| STAB2 | 0.005462 | −12.1125 | 0.005462 | 4.054075 |
| TP53RK | 0.032162 | −12.9236 | 0.032162 | 2.699438 |
| NRBP1 | 0.01371 | −13.0026 | 0.01371 | 3.373622 |
| WDR31 | 0.030212 | −13.2949 | 0.030212 | 2.849854 |
| GRIK5 | 0.007561 | −13.4168 | 0.007561 | 4.708967 |
| OR4K17 | 0.048117 | −13.4312 | 0.048117 | 2.396099 |
| TEAD3 | 0.018631 | −13.6944 | 0.018631 | 3.116448 |
| KCNC1 | 0.046743 | −13.9987 | 0.046743 | 2.478538 |
| TAL1 | 0.010966 | −14.5521 | 0.010966 | 3.441758 |
| FNDC3A | 0.021351 | −14.6464 | 0.021351 | 3.050709 |
| TTLL7 | 0.02278 | −15.0289 | 0.02278 | 4.314574 |
| IPO9 | 0.018373 | −15.4081 | 0.018373 | 3.086272 |
| CCNB1 | 0.012145 | −15.5935 | 0.012145 | 3.49297 |
| NEDD4 | 0.045106 | −16.3491 | 0.045106 | 2.778592 |
| PYCR1 | 0.008535 | −16.4759 | 0.008535 | 3.684416 |
| CHD5 | 0.013314 | −16.6688 | 0.013314 | 3.691435 |
| MAP3K7CL | 0.026452 | −16.8812 | 0.026452 | 3.342197 |
| LILRB4 | 0.01257 | −17.6547 | 0.01257 | 3.353677 |
| NPAT | 0.011577 | −17.9285 | 0.011577 | 3.444654 |
| CARD9 | 0.033435 | −17.9937 | 0.033435 | 2.643873 |
| NKIRAS2 | 0.016757 | −18.4647 | 0.016757 | 3.222583 |
| WASL | 0.013352 | −18.512 | 0.013352 | 3.471578 |
| MCOLN3 | 0.029765 | −18.6257 | 0.029765 | 2.724272 |
| NCF1 | 0.037576 | −18.6311 | 0.037576 | 2.875732 |
| KRTAP17-1 | 0.022265 | −18.6329 | 0.022265 | 2.922755 |
| TNFAIP8 | 0.042501 | −18.7949 | 0.042501 | 2.486498 |
| DDX10 | 0.023053 | −19.0278 | 0.023053 | 3.281868 |
| ANKH | 0.045883 | −19.2092 | 0.045883 | 2.593401 |
| SRSF7 | 0.025967 | −19.6254 | 0.025967 | 2.905782 |
| TFAP4 | 0.031157 | −19.8568 | 0.031157 | 2.724605 |
| ZC2HC1C | 0.027958 | −20.3924 | 0.027958 | 2.897824 |
| TRNT1 | 0.03368 | −21.0691 | 0.03368 | 2.718901 |
| OR2T6 | 0.01541 | −21.369 | 0.01541 | 3.184521 |
| OSER1-DT | 0.024334 | −21.8032 | 0.024334 | 3.230873 |
| MYF6 | 0.035479 | −22.1528 | 0.035479 | 2.726995 |
| CITED1 | 0.033499 | −22.6699 | 0.033499 | 2.6958 |
| TMEFF1 | 0.031101 | −23.0079 | 0.031101 | 2.837709 |
| GPR32 | 0.020997 | −23.4784 | 0.020997 | 2.96366 |
| MFNG | 0.013995 | −23.5909 | 0.013995 | 3.289209 |
| GGA3 | 0.043063 | −23.9837 | 0.043063 | 2.628317 |
| PNKP | 0.029952 | −24.4721 | 0.029952 | 2.834598 |
| ANGPTL2 | 0.018174 | −24.5954 | 0.018174 | 3.082329 |
| APEH | 0.04371 | −24.7358 | 0.04371 | 2.456789 |
| CDA | 0.024627 | −24.7446 | 0.024627 | 2.980324 |
| AKIP1 | 0.011293 | −24.9812 | 0.011293 | 3.524089 |
| RAB2A | 0.044598 | −25.5114 | 0.044598 | 2.592058 |
| LIN7C | 0.030767 | −25.5724 | 0.030767 | 2.839753 |
| KCNA2 | 0.005257 | −26.1932 | 0.005257 | 4.025905 |
| NOTCH2 | 0.021194 | −26.4841 | 0.021194 | 3.894322 |
| TADA3 | 0.0025 | −26.8045 | 0.0025 | 4.820992 |
| ZFYVE26 | 0.04555 | −27.0148 | 0.04555 | 2.504473 |
| SLC7A3 | 0.040911 | −27.2625 | 0.040911 | 2.508624 |
| NT5C1A | 0.032418 | −27.3029 | 0.032418 | 2.80156 |
| SMAD4 | 0.027338 | −28.3406 | 0.027338 | 2.781882 |
| RAI1 | 0.016138 | −28.4069 | 0.016138 | 4.06171 |
| SH3KBP1 | 0.042179 | −28.6198 | 0.042179 | 2.487558 |
| AP4B1 | 0.020353 | −28.8216 | 0.020353 | 3.172145 |
| RAB3A | 0.000282 | −29.1058 | 0.000282 | 6.876673 |
| PSG5 | 0.021616 | −29.1776 | 0.021616 | 3.044026 |
| CCT8 | 0.033975 | −29.4509 | 0.033975 | 2.632416 |
| ZNF132 | 0.039961 | −29.4959 | 0.039961 | 2.636202 |
| TAS2R3 | 0.010484 | −29.5048 | 0.010484 | 3.597519 |
| FGF23 | 0.017519 | −29.8677 | 0.017519 | 3.170501 |
| SMAD3 | 0.048166 | −30.1332 | 0.048166 | 2.83626 |
| SORCS1 | 0.042388 | −30.639 | 0.042388 | 2.590988 |
| DNAH2 | 0.041386 | −30.9753 | 0.041386 | 2.676474 |
| LAMC2 | 0.016256 | −31.0344 | 0.016256 | 3.408829 |
| PIGM | 0.037433 | −31.4677 | 0.037433 | 2.56216 |
| OMG | 0.01743 | −32.2304 | 0.01743 | 3.201692 |
| ZSWIM9 | 0.046248 | −32.6262 | 0.046248 | 2.576189 |
| CAPN9 | 0.015241 | −33.3042 | 0.015241 | 3.204873 |
| RND3 | 0.032772 | −33.4222 | 0.032772 | 2.655339 |
| SPART | 0.023464 | −33.6014 | 0.023464 | 2.885882 |
| AFF4 | 0.024188 | −33.6585 | 0.024188 | 3.049746 |
| C8G | 0.001844 | −33.7088 | 0.001844 | 5.661222 |
| BLM | 0.010209 | −34.0783 | 0.010209 | 3.617511 |
| PSAT1 | 0.015125 | −34.2154 | 0.015125 | 3.223176 |
| ARHGEF33 | 0.033116 | −34.23 | 0.033116 | 2.925322 |
| HADHA | 0.010181 | −34.3051 | 0.010181 | 3.772608 |
| SLC4A5 | 0.022909 | −35.3578 | 0.022909 | 2.909947 |
| PRSS12 | 0.021532 | −35.6109 | 0.021532 | 2.949108 |
| SYNE3 | 0.018969 | −35.8246 | 0.018969 | 3.198285 |
| GRIN2D | 0.035532 | −35.8302 | 0.035532 | 2.602798 |
| MTRNR2L5 | 0.018219 | −36.3496 | 0.018219 | 3.887153 |
| PICK1 | 0.034905 | −36.5526 | 0.034905 | 3.00624 |
| RHOV | 0.045151 | −36.7036 | 0.045151 | 2.640956 |
| USP41 | 0.013873 | −37.211 | 0.013873 | 3.435305 |
| CDK14 | 0.010511 | −37.5622 | 0.010511 | 3.709823 |
| TTLL1 | 0.041815 | −37.6593 | 0.041815 | 2.540624 |
| WSB2 | 0.03439 | −37.6749 | 0.03439 | 2.632581 |
| CPNE5 | 0.008387 | −37.7705 | 0.008387 | 3.956382 |
| RUBCNL | 0.016129 | −38.0109 | 0.016129 | 3.278906 |
| PMAIP1 | 0.001819 | −38.4019 | 0.001819 | 6.685734 |
| ZNF254 | 0.035291 | −38.9185 | 0.035291 | 2.61932 |
| FXYD3 | 0.015144 | −39.4483 | 0.015144 | 3.62472 |
| BIRC3 | 0.021339 | −39.5866 | 0.021339 | 2.972624 |
| IFIT3 | 0.003006 | −39.8003 | 0.003006 | 4.693241 |
| TNK2 | 0.017573 | −40.5289 | 0.017573 | 3.237353 |
| GIT1 | 0.00212 | −41.2699 | 0.00212 | 5.328246 |
| GMEB2 | 0.028546 | −42.0242 | 0.028546 | 2.791373 |
| DUSP19 | 0.033432 | −42.0288 | 0.033432 | 2.670806 |
| PTPRN2 | 0.024564 | −42.4322 | 0.024564 | 2.857323 |
| KNOP1 | 0.032013 | −42.4695 | 0.032013 | 2.669738 |
| UBE3D | 0.028141 | −42.7156 | 0.028141 | 2.816793 |
| EXT2 | 0.046655 | −42.7451 | 0.046655 | 2.44113 |
| SARS | 0.011701 | −42.8107 | 0.011701 | 3.383705 |
| RCL1 | 0.042782 | −43.0439 | 0.042782 | 2.475307 |
| CTSL | 0.014019 | −44.0177 | 0.014019 | 3.290611 |
| ACCSL | 0.034982 | −44.1635 | 0.034982 | 2.84988 |
| UGT2B15 | 0.008625 | −44.2281 | 0.008625 | 3.617838 |
| TAS2R4 | 0.035517 | −44.3783 | 0.035517 | 2.638624 |
| KRTAP8-1 | 0.037283 | −44.8928 | 0.037283 | 2.745139 |
| NCKAP1L | 0.031975 | −44.8979 | 0.031975 | 2.69263 |
| PCDHGA6 | 0.01247 | −44.9032 | 0.01247 | 3.340387 |
| KIRREL2 | 0.04951 | −45.4625 | 0.04951 | 2.397768 |
| CLDN17 | 0.049899 | −45.5465 | 0.049899 | 2.433153 |
| KCNE2 | 0.021854 | −45.6703 | 0.021854 | 2.942085 |
| VEZT | 0.002141 | −45.8199 | 0.002141 | 4.791025 |
| FZD2 | 0.041546 | −46.109 | 0.041546 | 2.596732 |
| TMEM204 | 7.15E−05 | −46.4388 | 7.15E−05 | 8.327786 |
| PJA2 | 0.035593 | −46.5698 | 0.035593 | 2.612977 |
| TUBA8 | 0.034215 | −46.8626 | 0.034215 | 2.67472 |
| TAS2R38 | 0.04019 | −46.9384 | 0.04019 | 2.618651 |
| MYT1 | 0.010301 | −46.9851 | 0.010301 | 3.855123 |
| PHKB | 0.016927 | −47.0816 | 0.016927 | 3.128525 |
| DDX18 | 0.027026 | −47.0855 | 0.027026 | 2.804641 |
| TAS2R43 | 0.027513 | −47.2356 | 0.027513 | 2.794764 |
| SMU1 | 0.00113 | −47.7144 | 0.00113 | 5.962681 |
| NDUFA3 | 0.005037 | −47.8915 | 0.005037 | 4.201426 |
| SNURF | 0.015555 | −48.0282 | 0.015555 | 3.226998 |
| RNF115 | 0.03516 | −48.2251 | 0.03516 | 2.855353 |
| VASN | 0.010819 | −48.3687 | 0.010819 | 3.451968 |
| GPAT4 | 0.00891 | −48.8168 | 0.00891 | 3.679557 |
| B3GNT7 | 0.034589 | −49.7637 | 0.034589 | 2.645374 |
| TOMM34 | 0.039038 | −49.9017 | 0.039038 | 2.537943 |
| CDKL3 | 0.045032 | −50.2116 | 0.045032 | 2.470052 |
| RPL12 | 0.010186 | −50.2639 | 0.010186 | 3.582396 |
| SYTL5 | 0.02887 | −50.3912 | 0.02887 | 2.928367 |
| MYD88 | 0.02908 | −50.8572 | 0.02908 | 2.807031 |
| LRRFIP2 | 0.029031 | −51.1889 | 0.029031 | 2.962101 |
| OR10J3 | 0.011656 | −51.3673 | 0.011656 | 3.724761 |
| DSCAML1 | 0.048215 | −52.6049 | 0.048215 | 2.585329 |
| SH3RF1 | 0.02149 | −52.9902 | 0.02149 | 3.193561 |
| ZFYVE1 | 0.027742 | −53.4913 | 0.027742 | 3.233376 |
| DNAJC28 | 0.046775 | −53.5979 | 0.046775 | 2.421096 |
| POU4F1 | 0.012787 | −53.669 | 0.012787 | 3.388904 |
| RAD50 | 0.019251 | −55.1071 | 0.019251 | 3.238089 |
| ORC5 | 0.00968 | −55.186 | 0.00968 | 3.523887 |
| MYH6 | 0.021058 | −55.2377 | 0.021058 | 3.032107 |
| CSRNP1 | 0.04727 | −55.5164 | 0.04727 | 2.501132 |
| ZFP3 | 0.030665 | −55.6563 | 0.030665 | 2.814442 |
| ALPI | 0.014515 | −56.1073 | 0.014515 | 3.5676 |
| TIGD2 | 0.02188 | −56.1403 | 0.02188 | 2.934995 |
| OR5I1 | 0.023599 | −56.247 | 0.023599 | 2.881825 |
| OR51B5 | 0.027898 | −56.2646 | 0.027898 | 2.948041 |
| ZNF84 | 0.001769 | −56.5232 | 0.001769 | 4.973171 |
| SS18L1 | 0.025282 | −56.5617 | 0.025282 | 3.125882 |
| CCNB1IP1 | 0.012521 | −57.345 | 0.012521 | 3.39883 |
| RPGRIP1 | 0.001847 | −57.4363 | 0.001847 | 5.046429 |
| SPTAN1 | 0.039649 | −57.437 | 0.039649 | 2.675296 |
| APOL3 | 0.014713 | −58.5319 | 0.014713 | 3.454972 |
| GCH1 | 0.030334 | −59.0075 | 0.030334 | 2.724573 |
| KLK1 | 0.012954 | −59.0599 | 0.012954 | 3.637467 |
| TRIM40 | 0.022928 | −59.2941 | 0.022928 | 2.908995 |
| HIST1H2BN | 0.042037 | −59.3479 | 0.042037 | 3.756523 |
| SPANXA1 | 0.033197 | −59.5841 | 0.033197 | 2.818645 |
| SLITRK2 | 0.04384 | −59.8857 | 0.04384 | 3.006612 |
| OR8H1 | 0.043045 | −60.0389 | 0.043045 | 2.637369 |
| VPS11 | 0.029083 | −60.1439 | 0.029083 | 2.925648 |
| NEU1 | 0.031394 | −60.8385 | 0.031394 | 2.689766 |
| ZNF274 | 0.016042 | −60.9138 | 0.016042 | 4.065912 |
| FBXO25 | 0.040923 | −60.9256 | 0.040923 | 2.65739 |
| PLA2R1 | 0.02498 | −60.9715 | 0.02498 | 3.0346 |
| NNMT | 0.022958 | −61.2017 | 0.022958 | 2.900868 |
| GMEB1 | 0.002372 | −61.5082 | 0.002372 | 4.654807 |
| CRABP1 | 0.040676 | −62.3236 | 0.040676 | 2.642382 |
| SLC24A2 | 0.045804 | −62.4707 | 0.045804 | 2.431944 |
| LIX1 | 0.007973 | −62.8643 | 0.007973 | 3.78336 |
| CCIN | 0.034178 | −63.2591 | 0.034178 | 2.710633 |
| DDX39B | 0.02185 | −64.1973 | 0.02185 | 3.285419 |
| PADI1 | 0.036388 | −64.4213 | 0.036388 | 2.794074 |
| THY1 | 0.004284 | −64.6132 | 0.004284 | 4.152477 |
| TNFRSF11B | 0.040473 | −64.9151 | 0.040473 | 2.569074 |
| H19 | 0.031972 | −65.0683 | 0.031972 | 2.679825 |
| ASPA | 0.043989 | −65.3985 | 0.043989 | 2.563485 |
| RAB29 | 0.02552 | −65.4185 | 0.02552 | 2.848687 |
| HMGB1 | 0.041978 | −65.7935 | 0.041978 | 2.661979 |
| NRAP | 0.014643 | −65.9539 | 0.014643 | 3.259948 |
| KIR3DL1 | 0.048409 | −66.6423 | 0.048409 | 2.422233 |
| SNX2 | 0.01441 | −66.7427 | 0.01441 | 3.590034 |
| TLE3 | 0.036306 | −66.7943 | 0.036306 | 2.747115 |
| CUZD1 | 0.014619 | −67.1236 | 0.014619 | 3.548514 |
| SEMA3F | 0.034725 | −67.3477 | 0.034725 | 2.782887 |
| S100B | 0.040472 | −67.5007 | 0.040472 | 2.529243 |
| CHRM5 | 0.018105 | −68.2075 | 0.018105 | 3.19298 |
| SRM | 0.007894 | −68.3074 | 0.007894 | 3.677033 |
| LPIN2 | 0.015066 | −68.86 | 0.015066 | 3.91616 |
| UCN3 | 0.045622 | −68.9446 | 0.045622 | 2.432127 |
| BEX1 | 0.009325 | −68.9927 | 0.009325 | 3.554561 |
| GNLY | 0.0491 | −69.4229 | 0.0491 | 2.46474 |
| H2AFY | 0.01141 | −69.4757 | 0.01141 | 3.536553 |
| UNC50 | 0.048034 | −69.7779 | 0.048034 | 2.56437 |
| PCYT1B | 0.013906 | −69.845 | 0.013906 | 3.646127 |
| GBX2 | 0.027552 | −69.9299 | 0.027552 | 2.773625 |
| EPHB1 | 0.001853 | −69.9688 | 0.001853 | 4.928555 |
| RFC5 | 0.031898 | −70.0573 | 0.031898 | 2.672254 |
| CA2 | 0.034193 | −70.5433 | 0.034193 | 2.63097 |
| SPOCK1 | 0.030173 | −71.5633 | 0.030173 | 2.850984 |
| EDDM3B | 0.002971 | −71.8129 | 0.002971 | 5.075179 |
| ZNF774 | 0.018958 | −71.8291 | 0.018958 | 3.171429 |
| SIGLEC1 | 0.038183 | −71.9439 | 0.038183 | 2.582216 |
| CDKL1 | 0.044499 | −72.4443 | 0.044499 | 2.444138 |
| ANP32A | 0.010134 | −72.6876 | 0.010134 | 3.683958 |
| AQP4 | 0.026073 | −72.9824 | 0.026073 | 2.986465 |
| IL10RB | 0.014974 | −73.5563 | 0.014974 | 3.226969 |
| MBD3L1 | 0.019648 | −73.7431 | 0.019648 | 3.028322 |
| AHSA1 | 0.005355 | −74.2494 | 0.005355 | 4.03429 |
| MBIP | 0.027316 | −75.5027 | 0.027316 | 2.843158 |
| PLXNB1 | 0.015897 | −75.6397 | 0.015897 | 3.16687 |
| EIF5 | 0.020862 | −76.0757 | 0.020862 | 3.095322 |
| GFRA2 | 0.015698 | −76.1055 | 0.015698 | 3.584495 |
| ARHGDIB | 0.035046 | −76.3162 | 0.035046 | 2.708169 |
| TMEM247 | 0.009224 | −76.4657 | 0.009224 | 3.599614 |
| UBE2E2 | 0.007249 | −76.6784 | 0.007249 | 3.836443 |
| KRTAP9-7 | 0.038788 | −76.9527 | 0.038788 | 2.612278 |
| CYP7A1 | 0.018963 | −77.7877 | 0.018963 | 3.121658 |
| RNF151 | 0.047701 | −78.2813 | 0.047701 | 2.511982 |
| ODF2L | 0.047265 | −78.466 | 0.047265 | 2.406861 |
| FBXL5 | 0.039874 | −79.2631 | 0.039874 | 2.608581 |
| CLDN8 | 0.049261 | −79.4667 | 0.049261 | 2.374754 |
| H1F0 | 0.044754 | −79.5403 | 0.044754 | 2.47925 |
| OSBPL5 | 0.040198 | −79.9699 | 0.040198 | 2.601803 |
| PURB | 0.016236 | −80.2955 | 0.016236 | 3.584724 |
| MYZAP | 0.031083 | −80.8328 | 0.031083 | 2.753776 |
| ABCB10 | 0.04086 | −82.0638 | 0.04086 | 2.6938 |
| DGKA | 0.047015 | −82.1815 | 0.047015 | 2.618424 |
| GJC2 | 0.000472 | −82.3696 | 0.000472 | 6.155897 |
| PDC | 0.026786 | −82.8115 | 0.026786 | 2.796434 |
| DNAJC8 | 0.012974 | −83.2797 | 0.012974 | 3.377905 |
| SRRM2 | 0.039596 | −83.7058 | 0.039596 | 2.57154 |
| MRPL47 | 0.013035 | −85.0765 | 0.013035 | 3.742601 |
| SPRY1 | 0.015907 | −85.6226 | 0.015907 | 3.161303 |
| AMMECR1 | 0.001322 | −85.7725 | 0.001322 | 6.265768 |
| PHYH | 0.028379 | −85.7783 | 0.028379 | 2.788707 |
| CBWD1 | 0.021562 | −85.9713 | 0.021562 | 3.028784 |
| MED6 | 0.030948 | −86.0542 | 0.030948 | 2.952104 |
| ST6GAL2 | 0.029862 | −86.6825 | 0.029862 | 2.763688 |
| DMXL2 | 0.009181 | −86.8748 | 0.009181 | 3.566198 |
| LRRC3 | 0.01026 | −86.9989 | 0.01026 | 3.601553 |
| ROPN1 | 0.004788 | −87.4182 | 0.004788 | 4.298454 |
| YES1 | 0.017505 | −88.7306 | 0.017505 | 4.372389 |
| OR52N1 | 0.016853 | −89.2741 | 0.016853 | 3.160598 |
| KRTAP4-3 | 0.025269 | −89.5428 | 0.025269 | 2.836281 |
| IL12RB2 | 0.036095 | −90.255 | 0.036095 | 2.647402 |
| TP53BP2 | 0.008674 | −90.6646 | 0.008674 | 4.023162 |
| PRM3 | 0.006209 | −90.8068 | 0.006209 | 3.921233 |
| PPM1J | 0.047901 | −91.2105 | 0.047901 | 2.544202 |
| MID2 | 0.046075 | −91.6253 | 0.046075 | 2.457269 |
| TRIM15 | 0.04733 | −92.3375 | 0.04733 | 2.524906 |
| ANGPT4 | 0.019486 | −92.839 | 0.019486 | 3.122176 |
| GPR45 | 0.033786 | −92.8868 | 0.033786 | 2.633284 |
| P3H2 | 0.031635 | −93.1718 | 0.031635 | 2.704553 |
| MRPS18C | 0.004018 | −93.4677 | 0.004018 | 4.679536 |
| OR5M8 | 0.040756 | −93.6206 | 0.040756 | 2.571832 |
| ADSSL1 | 0.030614 | −93.8993 | 0.030614 | 2.906841 |
| MCM3AP | 0.012023 | −93.9717 | 0.012023 | 3.71141 |
| SLC5A7 | 0.013021 | −94.7447 | 0.013021 | 3.4121 |
| PRL | 0.046411 | −95.6692 | 0.046411 | 2.424264 |
| RBCK1 | 0.020018 | −95.9503 | 0.020018 | 3.135711 |
| BTNL3 | 0.025521 | −96.0772 | 0.025521 | 2.893613 |
| AKAP10 | 0.032628 | −96.782 | 0.032628 | 2.744978 |
| WRN | 0.010579 | −96.93 | 0.010579 | 3.504632 |
| SUGT1P3 | 0.041012 | −97.6608 | 0.041012 | 2.510194 |
| PAIP2 | 0.025152 | −97.9142 | 0.025152 | 2.982914 |
| JPH2 | 0.049749 | −98.3974 | 0.049749 | 2.806806 |
| MAPRE1 | 0.027879 | −98.7989 | 0.027879 | 2.892559 |
| MAP1S | 0.047829 | −99.3068 | 0.047829 | 2.566694 |
| CBX3 | 0.020649 | −100.667 | 0.020649 | 3.010327 |
| FRG2 | 0.00659 | −100.794 | 0.00659 | 4.216055 |
| TCEA3 | 0.010027 | −100.807 | 0.010027 | 3.565413 |
| FPGS | 0.003202 | −101.036 | 0.003202 | 4.423121 |
| PMPCA | 0.027788 | −101.081 | 0.027788 | 2.901091 |
| TMEM74B | 0.04134 | −101.35 | 0.04134 | 2.621426 |
| COX7A2L | 0.011247 | −101.771 | 0.011247 | 3.47599 |
| KCNG4 | 0.040593 | −103.48 | 0.040593 | 2.632578 |
| TOM1 | 0.020414 | −103.951 | 0.020414 | 3.008028 |
| METTL23 | 0.045525 | −104.256 | 0.045525 | 2.651322 |
| GLP2R | 0.031604 | −104.527 | 0.031604 | 3.009122 |
| SERPINA6 | 0.002731 | −105.55 | 0.002731 | 4.821837 |
| YLPM1 | 0.003652 | −105.773 | 0.003652 | 4.318235 |
| MUC4 | 0.006524 | −106.36 | 0.006524 | 4.032407 |
| AP5S1 | 0.045084 | −106.389 | 0.045084 | 2.502779 |
| HBQ1 | 0.0137 | −107.195 | 0.0137 | 3.665258 |
| GEMIN5 | 0.003619 | −107.291 | 0.003619 | 4.303795 |
| TUBGCP2 | 0.004225 | −108.236 | 0.004225 | 4.201052 |
| COL11A1 | 0.031961 | −108.731 | 0.031961 | 2.679183 |
| KIT | 0.03209 | −109.817 | 0.03209 | 2.712621 |
| GMFB | 0.013297 | −110.252 | 0.013297 | 3.294855 |
| WDR5 | 0.039675 | −110.551 | 0.039675 | 2.530533 |
| REV3L | 0.031296 | −110.846 | 0.031296 | 2.765539 |
| ATOH7 | 0.031184 | −111.285 | 0.031184 | 2.942695 |
| C5AR1 | 0.042075 | −112.749 | 0.042075 | 2.654255 |
| TFDP1 | 0.048156 | −112.982 | 0.048156 | 2.520764 |
| PDPK1 | 0.025785 | −113.74 | 0.025785 | 3.011521 |
| SERF1A | 0.025512 | −114.81 | 0.025512 | 2.907026 |
| STK25 | 0.021467 | −115.768 | 0.021467 | 3.072336 |
| IL17RB | 0.00085 | −116.661 | 0.00085 | 5.567866 |
| RHEB | 0.009291 | −116.928 | 0.009291 | 3.55423 |
| CCR10 | 0.03876 | −118.572 | 0.03876 | 2.558267 |
| DNAJC30 | 0.025283 | −118.639 | 0.025283 | 2.837623 |
| IL20RA | 0.003252 | −118.644 | 0.003252 | 4.454366 |
| GGN | 0.007844 | −118.872 | 0.007844 | 5.362481 |
| LRP8 | 0.007742 | −119.468 | 0.007742 | 3.971134 |
| RXFP4 | 0.043458 | −119.505 | 0.043458 | 2.467621 |
| GALNT9 | 0.03914 | −119.894 | 0.03914 | 2.627414 |
| ALDH2 | 0.042357 | −120.192 | 0.042357 | 2.532243 |
| TRPC3 | 0.004645 | −120.329 | 0.004645 | 4.528089 |
| PGM1 | 0.000553 | −120.666 | 0.000553 | 6.028762 |
| EMILIN2 | 0.00964 | −121.167 | 0.00964 | 3.999281 |
| NCK2 | 0.049316 | −121.435 | 0.049316 | 2.485775 |
| GPC2 | 0.007902 | −121.77 | 0.007902 | 3.683829 |
| UBE2M | 0.045013 | −122.158 | 0.045013 | 2.887497 |
| NAA20 | 0.030301 | −122.54 | 0.030301 | 2.716875 |
| IL12A | 0.003271 | −123.628 | 0.003271 | 4.378596 |
| RN7SL1 | 0.027044 | −126.835 | 0.027044 | 3.036598 |
| DRAP1 | 0.027782 | −127.862 | 0.027782 | 2.87356 |
| RAI14 | 0.012537 | −128.455 | 0.012537 | 3.683338 |
| RIOK1 | 0.00927 | −128.572 | 0.00927 | 3.583574 |
| ZNF671 | 0.010617 | −128.611 | 0.010617 | 3.500507 |
| C22orf15 | 0.030653 | −131.491 | 0.030653 | 2.927605 |
| SNAI1 | 0.0014 | −131.574 | 0.0014 | 5.129397 |
| IL1B | 0.008431 | −134.211 | 0.008431 | 3.698232 |
| PGD | 0.033675 | −137.199 | 0.033675 | 2.743656 |
| MBP | 0.001012 | −137.732 | 0.001012 | 5.724071 |
| RIMS1 | 0.048458 | −138.818 | 0.048458 | 2.474987 |
| POLR1E | 0.022061 | −139.036 | 0.022061 | 3.631181 |
| LEFTY2 | 0.038525 | −139.557 | 0.038525 | 2.747422 |
| ARHGAP1 | 0.004747 | −140.469 | 0.004747 | 4.076045 |
| SNAP91 | 0.004027 | −140.557 | 0.004027 | 4.749817 |
| NRL | 0.037805 | −141.416 | 0.037805 | 2.793558 |
| RPL31 | 0.022647 | −142.192 | 0.022647 | 3.195502 |
| SFTA3 | 0.006118 | −143.199 | 0.006118 | 4.044451 |
| CD3D | 0.034816 | −143.252 | 0.034816 | 3.654446 |
| PMS2P3 | 0.017893 | −143.741 | 0.017893 | 3.202071 |
| NKX2-8 | 0.048995 | −145.519 | 0.048995 | 2.405604 |
| CYP1B1 | 0.018912 | −145.613 | 0.018912 | 3.038598 |
| RPS6KA6 | 0.004303 | −148.126 | 0.004303 | 4.340966 |
| POP7 | 0.017328 | −149.152 | 0.017328 | 3.301923 |
| CTSW | 0.020237 | −149.626 | 0.020237 | 2.993515 |
| SLC6A2 | 0.022133 | −150.092 | 0.022133 | 3.218885 |
| IRAK3 | 0.013725 | −150.534 | 0.013725 | 3.49122 |
| BARHL1 | 0.025853 | −150.869 | 0.025853 | 2.927118 |
| DGKQ | 0.033977 | −151.586 | 0.033977 | 2.629823 |
| CYYR1 | 0.002589 | −152.011 | 0.002589 | 5.001182 |
| CNNM1 | 0.047087 | −152.813 | 0.047087 | 2.576327 |
| MAP1B | 0.025006 | −153.422 | 0.025006 | 2.897623 |
| ZMYND11 | 0.001093 | −154.852 | 0.001093 | 5.468477 |
| KRTAP16-1 | 0.017525 | −156.158 | 0.017525 | 3.217043 |
| PSMC4 | 0.008791 | −156.999 | 0.008791 | 3.731448 |
| DPYSL4 | 0.048963 | −157.302 | 0.048963 | 2.455984 |
| ACOT8 | 0.047884 | −159.848 | 0.047884 | 2.459586 |
| ZNF316 | 0.023666 | −160.801 | 0.023666 | 2.994667 |
| CLDN7 | 0.006027 | −162.342 | 0.006027 | 4.343622 |
| UGT1A8 | 0.045697 | −163.252 | 0.045697 | 2.494876 |
| THPO | 0.046122 | −163.47 | 0.046122 | 2.436522 |
| FBLN2 | 0.031243 | −164.447 | 0.031243 | 2.878908 |
| UBP1 | 0.005652 | −165.419 | 0.005652 | 4.09233 |
| ADAM6 | 0.026416 | −165.527 | 0.026416 | 2.806704 |
| PRLHR | 0.020828 | −166.058 | 0.020828 | 3.176899 |
| PCGF1 | 0.026945 | −166.558 | 0.026945 | 3.588054 |
| DHRS11 | 0.041948 | −166.777 | 0.041948 | 2.505046 |
| CCDC78 | 0.025547 | −168.023 | 0.025547 | 2.857946 |
| TAF9 | 0.000443 | −168.984 | 0.000443 | 6.207143 |
| CNN3 | 0.021677 | −169.585 | 0.021677 | 3.245513 |
| B3GALT4 | 0.030956 | −169.607 | 0.030956 | 3.149113 |
| TYMP | 0.002557 | −169.874 | 0.002557 | 5.555771 |
| CDKN2B | 0.04477 | −169.878 | 0.04477 | 2.597175 |
| OR4C6 | 0.003688 | −170.159 | 0.003688 | 4.395451 |
| MPZ | 0.028611 | −172.312 | 0.028611 | 2.772487 |
| RANBP1 | 0.019092 | −172.461 | 0.019092 | 3.031234 |
| GNAT1 | 0.009614 | −173.349 | 0.009614 | 3.77663 |
| UBAP1 | 0.016274 | −173.633 | 0.016274 | 3.528958 |
| TAS2R16 | 0.041002 | −174.34 | 0.041002 | 2.536929 |
| CHRNA2 | 0.048361 | −176.219 | 0.048361 | 2.550192 |
| OR11H6 | 0.015586 | −176.791 | 0.015586 | 3.268493 |
| NDST3 | 0.039737 | −181.439 | 0.039737 | 3.487183 |
| LSM1 | 0.026841 | −181.549 | 0.026841 | 2.879285 |
| ABT1 | 0.00089 | −183.277 | 0.00089 | 5.787554 |
| PALMD | 0.00327 | −183.536 | 0.00327 | 6.197588 |
| NXF5 | 0.000498 | −185.888 | 0.000498 | 6.888045 |
| TIGD1 | 0.011228 | −188.252 | 0.011228 | 3.520109 |
| PTX3 | 0.033992 | −189.071 | 0.033992 | 2.83393 |
| GRM8 | 0.007049 | −189.36 | 0.007049 | 4.306399 |
| RAMP3 | 0.039426 | −190.918 | 0.039426 | 2.592205 |
| PPP6C | 0.004437 | −199.452 | 0.004437 | 4.367913 |
| DHCR24 | 0.007156 | −201.487 | 0.007156 | 4.565597 |
| GRPR | 0.001325 | −202.185 | 0.001325 | 5.213724 |
| PLG | 0.014734 | −203.064 | 0.014734 | 3.219308 |
| CELA1 | 0.03869 | −204.606 | 0.03869 | 2.546206 |
| COLQ | 0.014053 | −205.334 | 0.014053 | 3.908916 |
| HTN3 | 0.045798 | −205.334 | 0.045798 | 2.634803 |
| UBAC2 | 0.024631 | −208.066 | 0.024631 | 2.864824 |
| IL20 | 0.004374 | −209.417 | 0.004374 | 5.402087 |
| MYL12A | 0.022623 | −212.285 | 0.022623 | 3.006428 |
| PLA2G5 | 0.011964 | −213.006 | 0.011964 | 3.780673 |
| CLASP2 | 0.022 | −214.563 | 0.022 | 3.223544 |
| FAM118A | 0.002642 | −219.689 | 0.002642 | 5.30095 |
| KIFC1 | 0.015629 | −221.663 | 0.015629 | 3.455516 |
| AK7 | 0.043028 | −221.879 | 0.043028 | 2.466935 |
| FSHR | 0.015577 | −223.647 | 0.015577 | 3.315497 |
| COQ8B | 0.020963 | −225.319 | 0.020963 | 3.296656 |
| CLN3 | 0.030308 | −225.951 | 0.030308 | 3.141936 |
| KLHL11 | 0.03745 | −226.302 | 0.03745 | 2.625147 |
| UBE2I | 0.002152 | −231.252 | 0.002152 | 4.803289 |
| KCTD12 | 0.029607 | −233.764 | 0.029607 | 2.970054 |
| RPL18 | 0.037347 | −236.554 | 0.037347 | 2.568383 |
| ITPR1 | 0.035912 | −239.909 | 0.035912 | 2.708433 |
| OR13G1 | 0.035402 | −240.236 | 0.035402 | 2.782988 |
| PECAM1 | 0.020379 | −240.419 | 0.020379 | 2.98701 |
| EFNB1 | 0.014057 | −241.631 | 0.014057 | 3.254762 |
| DBH | 0.017119 | −243.454 | 0.017119 | 3.117771 |
| BPIFB2 | 0.001985 | −243.503 | 0.001985 | 4.794896 |
| GSTA4 | 0.001402 | −247.307 | 0.001402 | 5.237646 |
| P2RY4 | 0.045217 | −249.002 | 0.045217 | 2.489161 |
| BCL2L12 | 0.016055 | −252.584 | 0.016055 | 3.214729 |
| DAZAP1 | 0.008419 | −255.27 | 0.008419 | 3.904787 |
| CDCA3 | 0.031405 | −256.834 | 0.031405 | 2.852423 |
| CALCA | 0.021099 | −260.49 | 0.021099 | 3.015484 |
| ATE1 | 0.000605 | −263 | 0.000605 | 6.7525 |
| OR4K14 | 0.001503 | −263.189 | 0.001503 | 5.07833 |
| CALU | 0.037266 | −268.063 | 0.037266 | 2.602473 |
| MAPK10 | 0.004041 | −277.579 | 0.004041 | 4.430004 |
| ADAM18 | 0.020382 | −280.309 | 0.020382 | 3.21359 |
| LPAR3 | 0.025273 | −280.373 | 0.025273 | 3.868234 |
| DAPP1 | 0.035872 | −280.387 | 0.035872 | 2.596615 |
| BCR | 0.040455 | −281.723 | 0.040455 | 2.50911 |
| FAM189B | 0.038106 | −287.052 | 0.038106 | 2.598172 |
| HAUS2 | 0.011688 | −289.599 | 0.011688 | 3.544907 |
| TXN | 0.035665 | −289.758 | 0.035665 | 2.613429 |
| HS3ST3A1 | 0.04141 | −291.82 | 0.04141 | 2.618218 |
| CRP | 0.042775 | −294.168 | 0.042775 | 2.638156 |
| HAGH | 0.045048 | −296.179 | 0.045048 | 2.60287 |
| RPS16 | 0.028136 | −298.205 | 0.028136 | 2.784628 |
| NDUFA13 | 0.010903 | −298.583 | 0.010903 | 3.467322 |
| TRPM7 | 0.023294 | −300.384 | 0.023294 | 2.89339 |
| GLRX2 | 0.030123 | −305.388 | 0.030123 | 2.852131 |
| NIPSNAP1 | 0.032691 | −307.07 | 0.032691 | 2.703183 |
| LAG3 | 0.023669 | −308.809 | 0.023669 | 3.485134 |
| PAK1 | 0.009492 | −311.349 | 0.009492 | 3.548774 |
| DGKI | 0.006002 | −312.256 | 0.006002 | 4.144157 |
| MLLT11 | 0.020225 | −315.486 | 0.020225 | 4.108678 |
| LRRN3 | 0.003394 | −318.938 | 0.003394 | 4.373126 |
| PTPRK | 0.039662 | −335.301 | 0.039662 | 3.898156 |
| BCL9 | 0.039765 | −339.086 | 0.039765 | 2.686741 |
| SEMA4F | 0.027447 | −345.348 | 0.027447 | 2.815463 |
| OR5H6 | 0.005628 | −345.466 | 0.005628 | 4.275493 |
| ZNF263 | 0.028604 | −346.838 | 0.028604 | 2.833814 |
| ELAC1 | 0.049559 | −347.282 | 0.049559 | 2.52836 |
| KCNAB2 | 0.017946 | −348.687 | 0.017946 | 3.643548 |
| HBG1 | 0.005265 | −350.942 | 0.005265 | 3.993474 |
| LMNB2 | 0.046283 | −361.636 | 0.046283 | 2.623961 |
| HMGN4 | 0.014638 | −362.36 | 0.014638 | 3.266401 |
| PROP1 | 0.006073 | −373.314 | 0.006073 | 3.880691 |
| STK38L | 0.003765 | −376.292 | 0.003765 | 4.258779 |
| EML1 | 0.000835 | −377.003 | 0.000835 | 5.733649 |
| ARHGEF1 | 0.035568 | −378.793 | 0.035568 | 2.597471 |
| HLA-DMB | 0.02846 | −379.164 | 0.02846 | 2.846894 |
| NCOA1 | 0.038316 | −380.958 | 0.038316 | 2.64274 |
| IL27 | 0.01352 | −381.483 | 0.01352 | 3.686383 |
| PPP1R3C | 0.015804 | −381.623 | 0.015804 | 3.16634 |
| KRTAP3-3 | 0.000546 | −381.984 | 0.000546 | 6.354808 |
| PELI1 | 0.014618 | −384.875 | 0.014618 | 3.657821 |
| GSPT2 | 0.001354 | −394.542 | 0.001354 | 5.236618 |
| TNFSF12 | 0.015897 | −395.289 | 0.015897 | 3.293768 |
| RGS7 | 0.029187 | −400.104 | 0.029187 | 2.798724 |
| LRRTM2 | 0.016078 | −408.229 | 0.016078 | 3.183535 |
| SLC38A6 | 0.013277 | −421.016 | 0.013277 | 3.293388 |
| C22orf23 | 0.003237 | −428.232 | 0.003237 | 5.304203 |
| IL22RA2 | 0.001683 | −435.634 | 0.001683 | 5.546508 |
| RPS29 | 0.025249 | −438.292 | 0.025249 | 2.887054 |
| MT4 | 0.012562 | −439.664 | 0.012562 | 3.391609 |
| IRX2 | 0.0115 | −445.922 | 0.0115 | 3.414092 |
| PARP1 | 0.007258 | −446.127 | 0.007258 | 3.752143 |
| NRG2 | 0.008927 | −459.384 | 0.008927 | 3.647686 |
| AFF2 | 0.015799 | −459.882 | 0.015799 | 3.401595 |
| ATG2B | 0.000666 | −470.575 | 0.000666 | 5.834744 |
| PLA2G3 | 0.010908 | −471.515 | 0.010908 | 4.961982 |
| STK3 | 0.013499 | −473.677 | 0.013499 | 3.284371 |
| FKBP5 | 0.041598 | −475.778 | 0.041598 | 2.709601 |
| RENBP | 0.041898 | −480.762 | 0.041898 | 2.526555 |
| INGX | 0.035492 | −483.012 | 0.035492 | 2.608703 |
| WNT4 | 0.028983 | −497.901 | 0.028983 | 2.820323 |
| RAB3D | 0.046909 | −504.045 | 0.046909 | 2.815674 |
| RHOF | 0.012936 | −505.767 | 0.012936 | 3.32777 |
| RAB40A | 0.035081 | −508.732 | 0.035081 | 2.781914 |
| ERAS | 0.024438 | −562.473 | 0.024438 | 2.857757 |
| CLNK | 0.017978 | −563.793 | 0.017978 | 3.075764 |
| ITGAE | 0.018369 | −589.697 | 0.018369 | 3.189301 |
| NCOA5 | 0.00027 | −594.971 | 0.00027 | 7.169114 |
| AVPR1B | 0.043129 | −596.689 | 0.043129 | 2.688463 |
| TH | 0.029105 | −597.708 | 0.029105 | 3.040735 |
| CRYGC | 0.035896 | −680.35 | 0.035896 | 2.591038 |
| STH | 0.020416 | −682.851 | 0.020416 | 3.053051 |
| MRPL48 | 0.018757 | −690.073 | 0.018757 | 3.372864 |
| MAPK11 | 0.002697 | −690.596 | 0.002697 | 4.846385 |
| SIPA1L1 | 0.001558 | −693.972 | 0.001558 | 7.155197 |
| RPL14 | 0.007822 | −697.109 | 0.007822 | 3.787336 |
| HSD17B3 | 0.001911 | −698.662 | 0.001911 | 4.826678 |
| NDUFA5 | 0.037919 | −710.641 | 0.037919 | 2.639772 |
| EIF3J | 0.006262 | −721.708 | 0.006262 | 5.467907 |
| PPBP | 0.023343 | −726.85 | 0.023343 | 3.158226 |
| SNAP25 | 0.046276 | −770.954 | 0.046276 | 2.455737 |
| MYBBP1A | 0.013023 | −779.706 | 0.013023 | 3.454482 |
| ATOH1 | 0.046939 | −868.465 | 0.046939 | 2.502853 |
| GOLGA6L22 | 0.020984 | −1051.19 | 0.020984 | 3.320034 |
| APLP2 | 0.00613 | −1084.29 | 0.00613 | 4.168659 |
| FCRLA | 0.016047 | −1099.79 | 0.016047 | 3.232175 |
| SPNS1 | 0.017095 | −1124.91 | 0.017095 | 3.422234 |
| ACIN1 | 0.045951 | −1167.7 | 0.045951 | 2.49058 |
| HSD17B2 | 0.013757 | −1204.4 | 0.013757 | 3.547623 |
| PLA2G7 | 0.008217 | −1264.28 | 0.008217 | 4.704401 |
| SMARCD3 | 0.039101 | −1325.16 | 0.039101 | 2.552594 |
| PSMB9 | 0.040648 | −1563.73 | 0.040648 | 2.729409 |
| FAM50B | 0.03015 | −1565.06 | 0.03015 | 2.722168 |
| EBNA1BP2 | 0.039448 | −1998.13 | 0.039448 | 3.897626 |
| LZTR1 | 0.001414 | −2075.01 | 0.001414 | 5.762291 |
| SMIM6 | 0.015682 | −4008.18 | 0.015682 | 3.287791 |
| C11orf53 | 0.044196 | −135753 | 0.044196 | 2.567963 |

1. A method of inducing biostasis in a tissue or organ, the method comprising contacting the tissue or organ in need of preservation with an agent that alters the function of at least one ion channel selected from the group consisting of EAAT1 ion channel and NCX1 ion channel, wherein the contacted tissue or organ exhibits biostasis.

2. A method of tissue or organ transplant, the method comprising contacting a donor tissue or organ in situ or ex vivo with an agent that alters the function of at least one ion channel selected from the group consisting of EAAT1 ion channel and NCX1 ion channel.

3. The method of claim 1, wherein the agent further activates the δ-opioid receptor following contact.

4. The method of claim 1, wherein the agent does not activate the δ-opioid receptor following contact.

5. (canceled)

6. (canceled)

7. The method of claim 1, wherein altering the function is inhibiting, slowing, or activating the function.

8. The method of claim 1, wherein the tissue is selected from the group consisting of cornea, bone, tendon, pancreas islet, heart valve, nerve, vascular, deep tissue flap, fat tissue, muscle, and vein.

9. The method of claim 1, wherein the organ is selected from the group consisting of intestine, stomach, heart, kidney, bladder, pancreas, liver, lung, brain, skin, uterus, digit, and limb.

10. The method of claim 1, wherein the contacting suppresses the metabolism or induces biostasis of the tissue or organ.

11. The method of claim 1, wherein the agent is SNC-80 or donepezil, or
a derivative, analog, or variant of SNC-80 or donepezil that alters the function of at least one ion channel selected from the group consisting of EAAT1 ion channel and NCX1 ion channel.

12.-15. (canceled)

16. The method of claim 1, further comprising contacting with at least a second agent that alters the function of at least one ion channel selected from the group consisting of EAAT1 ion channel and NCX1 ion channel.

17. The method of claim 16, wherein the agonist or agent and the at least second agent are contacted at substantially the same time or at different times.

18. The method of claim 16, wherein the at least second agent is an inhibitor of the NCX1 ion channel.

19. The method of claim 18, wherein the inhibitor is KB-R7943 mesylate.

20. The method of claim 1, wherein the agent or agonist is comprised in a vehicle that is deuterium oxide.

21. The method of claim 1, wherein the contacting is a single contact, or reoccurring contacting.

22. The method of claim 1, wherein the contacting is performed for at least 1 hour, 2 hours, 3 hours, 4 hours, 5 hours, 6 hours, 10 hours, 18 hours, 24 hours, 36 hours 48, hour, 96 hours or more.

23. The method of claim 1, further comprising contacting the tissue or organ with at least a second, biostatic compound.

24. The method of claim 1, wherein the at least a second compound is selected from the group consisting of hydrogen sulfide, nitrogen, argon, Oligomycin A, rotenone, 2-deoxyglucose, adenosine monophosphate (AMP), a neuropeptide, deferoxamine, and a prolyl hydroxylase inhibitor.

25.-30. (canceled)

31. A composition comprising at least two agents that alter the function of at least one ion channel selected from the group consisting of EAAT1 ion channel and NCX1 ion channel.

32. (canceled)

33. The composition of claim 31, further comprising deuterium oxide.

34. (canceled)

35. (canceled)
